(19) United States
(12) Patent Application Publication
(10) Pub. No.: US 2023/0183234 A1
(43) Pub. Date: Jun. 15, 2023

(54) IAP ANTAGONIST COMPOUNDS AND INTERMEDIATES AND METHODS FOR SYNTHESIZING THE SAME

(71) Applicant: Otsuka Pharmaceutical Co., Ltd., Tokyo (JP)

(72) Inventors: Prabhudas BODHURI, San Ramon, CA (US); Hem Raj KHATRI, PLeasanton, CA (US); Nipun DAVAR, PLeasanton, CA (US); Ian SCOTT, PLeasanton, CA (US)

(21) Appl. No.: 17/923,188

(22) PCT Filed: May 3, 2021
(86) PCT No.: PCT/US2021/030456

§ 371 Date: Nov. 3, 2022

Related U.S. Application Data
(60) Provisional application No. 63/019,874, filed on May 4, 2020.
(60) Provisional application No. 63/019,865, filed on May 4, 2020.

(51) Int. Cl. C07D 471/04; C07D 413/06; C07D 213/61; C07D 241/04; C07F 5/04

(52) U.S. Cl. CPC C07D 471/04; C07D 413/06; C07D 213/61; C07D 241/04; C07F 5/04

(57) ABSTRACT

A compound of formula (XXIII) and methods for preparation thereof are provided (XXIII).

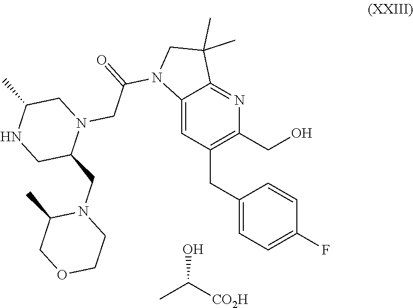

### CROSS REFERENCE TO RELATED APPLICATIONS

This application claims the benefit under 35 U.S.C. § 119(e) of U.S. Provisional Application No. 63/019,865 filed May 4, 2020, and U.S. Provisional Application No. 63/019,874 filed May 4, 2020, which are hereby incorporated by reference herein in their entireties.

### FIELD

The present application relates to improved IAP antagonist compounds and intermediates and methods for synthesizing the same.

### BACKGROUND

Apoptosis, or programmed cell death, is a vital component of human physiology and normal immune response. Insufficient or excessive apoptosis can cause human disease, including neurodegenerative diseases, autoimmune disorders and many types of cancer. Inhibitors of apoptosis (IAPs) are expressed in several cancers, such as lymphoma. Eight distinct human IAPs have been characterized: XIAP, hILP-2, c-IAP1, c-IAP2, ML-IAP, NAIP, Survivin and Apollon. Mary X. D. O'Riordan, Laura D. Bauler, Fiona L. Scott, and Colin S. Duckett, “Inhibitor of apoptosis proteins in eukaryotic evolution and development: a model of thematic conservation”, Developmental Cell 15(4): 497-508, October 2008. The common structural features, mechanisms and expression of IAPs in cancer are described in U.S. Pat. No. 9,783,538, which is incorporated by reference in its entirety herein.

Improved IAP antagonist compounds and improved intermediates and methods for synthesizing IAP antagonist compounds are disclosed herein.

### SUMMARY

Provided herein are intermediates and methods for preparing a compound of formula (XXII) and (XXIII), the compounds are described below and in U.S. Pat. No. 9,783,538.

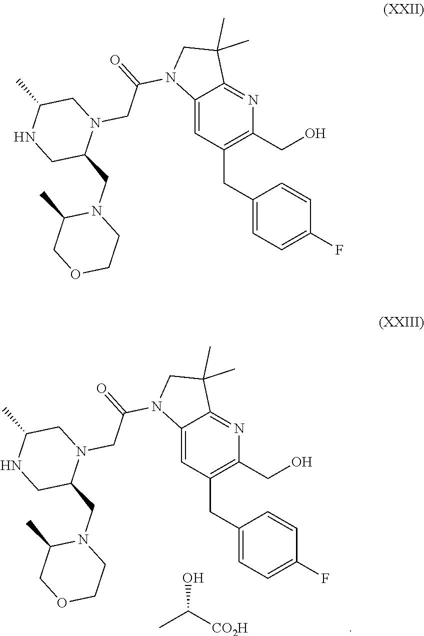

The present application provides compounds of formulas (Ia), (Va), (VII), (IX), (XXIII) and (XXIIIa) and methods of synthesizing compounds of formulas (Ia), (Va), (VII), (IX), (XXIII) and (XXIIIa). The compound of formula (IX) is an intermediate in synthesis of compounds of formulas (XXIII) and (XXIIIa). The present application provides compounds of formulas (I), (XVI), (XVIa) and (XX) and (XXIIIa) and methods of synthesizing compounds of formulas (I), (XVI), (XVIa) and (XX). The compounds of formulas (XXIII) and (XXIIIa) are antagonists of the IAP family of proteins, and especially XIAP, and/or cIAP (such as cIAP1 and/or cIAP2) and are useful in the treatment of IAP-mediated conditions.

In one aspect, provided herein is a method for the preparation of compound (XXIII) comprising contacting compound (XX) with compound (XIII) to obtain compound (XXI) and converting compound (XXI) to compound (XXIII), as described below in the detailed description section.

In another aspect, provided herein is a method for the preparation of compound (XXIII) comprising converting compound (IX) to compound (X), then converting compound (X) to compound (XIII) and contacting compound (XX) with compound (XIII) to obtain compound (XXI), then converting compound (XXI) to compound (XXIII), as described below in the detailed description section.

In a further aspect, provided herein are methods for preparing compound (IX) as described below in the detailed description section.

In additional aspects are provided compounds of formulas (Ia), (I), (XVIa), (IX), (X), (XI), (XX), (IIIa) and (Vb) as described herein.

The compounds of formulas (Ia), (Va), (VII) and (IX) are useful for synthesizing compounds of formula (XXIIIa) or a tautomeric, a stereochemically isomeric form, a pharmaceutically acceptable salt or a solvate thereof:

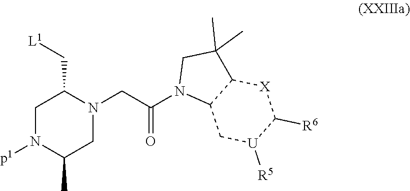

wherein X, U, R⁵, R⁶, L¹, L², and P¹ are defined as disclosed in U.S. Pat. No. 9,783,538.

In an exemplary embodiment, methods of synthesizing compounds of formula (XXIIIa), a tautomeric, a stereochemically isomeric form, a pharmaceutically acceptable salt or a solvate thereof and methods of synthesizing compounds of formula (XXIIIa) a tautomeric, a stereochemically isomeric form, a pharmaceutically acceptable salt or a solvate thereof with compounds of formulas (I), (XVI), (XVIa) and (XX) are provided:

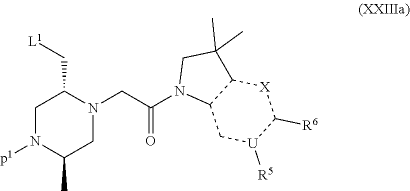

wherein X, U, R⁵, R⁶, L¹, L², and P¹ are defined as disclosed in U.S. Pat. No. 9,783,538 incorporated by reference herein.

The compound of formula (XXIIIa) produced by the embodiments and methods of synthesis disclosed herein is for use in the prophylaxis or treatment of a disease or condition and in formulations and pharmaceutical compositions comprising a compound of formula (XXIIIa) as described by U.S. Pat. No. 9,783,538 incorporated by reference in its entirety herein.

The foregoing and other objects, features and advantages of the present disclosure will become more readily apparent from the following detailed description of exemplary embodiments as disclosed herein.

### BRIEF DESCRIPTION OF THE DRAWINGS

Embodiments of the present application are described, by way of example only, with reference to the attached Figures, wherein:

FIG. 1 depicts an exemplary synthesis of the compound of formula (VIII);

FIG. 2 depicts an exemplary synthesis of the compound of formula (XXIII);

FIG. 3 depicts an exemplary synthesis of the compound of formula (VII);

FIG. 4 depicts an exemplary synthesis of the compound of formula (IX);

FIG. 5 depicts X-Ray Powder Diffraction of Form C of the compound of formula (XXIII);

FIG. 6 is a graph depicting the effect of palladium content on area % of RRT 1.3 (25° C./60% RH) of the compound of formula (XXIII); and

FIG. 7 is a graph depicting the effect of temperature on level of impurity at RRT 1.3 in APIs made from the compound of formula (XXIII).

### DETAILED DESCRIPTION

The following examples and embodiments disclosed and described in this application are illustrative. Those of ordinary skill in the art will understand that various alterations to the embodiments may exist without departing from the scope or intent of the present application or exemplary embodiments disclosed, including variations with respect to the synthesis methods, processes, reactants, reagents, parameters and conditions described herein. The present application is directed to improved methods, reactants and reagents for synthesizing the compounds of formulas (Ia), (Va), (VII), (IX), (XXIII) and (XXIIIa).

The present application provides compounds of formulas (I), (XVI), (XVIa) and (XX) and methods of synthesizing the compounds of formulas (I), (XVI), (XVIa) and (XX). The compounds of formulas (I), (XVI), (XVIa) and (XX) are useful for synthesizing compounds of formula (XXIIIa). The compounds of formula (XXIIIa) are antagonists of the IAP family of proteins, and especially XIAP, and/or cIAP (such as cIAP1 and/or cIAP2) and are useful in the treatment of IAP-mediated conditions.

### Definitions

As used in the present specification, the following words and phrases are generally intended to have the meanings as set forth below, except to the extent that the context in which they are used indicates otherwise.

The term “comprise” and variations thereof, such as, “comprises” and “comprising” are to be construed in an open, inclusive sense, that is, as “including, but not limited to.” Further, the singular forms “a,” “an,” and “the” include plural references unless the context clearly dictates otherwise.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±2.5%. In certain other embodiments, the term “about” includes the indicated amount ±1%. Also, a term such as “about X” includes description of “X”.

Recitation of numeric ranges of values throughout the disclosure is intended to serve as a shorthand notation of referring individually to each separate value falling within the range inclusive of the values defining the range, and each separate value is incorporated in the specification as it were individually recited herein.

“Alkyl,” by itself, or as part of another substituent, means, unless otherwise stated, a straight or branched chain hydrocarbyl group, having the number of carbon atoms designated (i.e. C₁-C₆ means one to six carbons). Representative alkyl groups include straight and branched chain alkyl groups having 1, 2, 3, 4, 5, 6, 7, 8, 9, 10, 11 or 12 carbon atoms. Further representative alkyl groups include straight and branched chain alkyl groups having 1, 2, 3, 4, 5, 6, 7 or 8 carbon atoms. Examples of alkyl groups include methyl, ethyl, n-propyl, isopropyl, n-butyl, t-butyl, isobutyl, sec-butyl, n-pentyl, n-hexyl, n-heptyl, n-octyl, and the like.

“Aryl” by itself, or as part of another substituent, unless otherwise stated, refers to a monocyclic, bicyclic or polycyclic polyunsaturated aromatic hydrocarbon radical containing 6 to 14 ring carbon atoms, which can be a single ring or multiple rings (up to three rings) which are fused together or linked covalently. Non-limiting examples of unsubstituted aryl groups include phenyl, 1-naphthyl and 2-naphthyl. The term “arylene” refers to a divalent aryl, wherein the aryl is as defined herein.

“Boc” refers to a tert-butyloxy carbonyl group.

“Ph” refers to a phenyl group.

“Protecting group” refers to a moiety that masks a reactive group. By of example only, in some embodiments, protecting groups include and are not limited to tert-butyloxycarbonyl (Boc), carbobenzoxy (Cbz), benzyl, para-methoxy benzyl, para-nitro benzyl, or any other protecting group described in Protective Groups in Organic Synthesis 4th Edition by P. G. M. Wuts and T. W. Greene.

In many cases, the compounds of this disclosure are capable of forming acid and/or base salts by virtue of the presence of amino and/or hydroxy groups or groups similar thereto. Salts include, for example, salts with inorganic acids and salts with an organic acid. In addition, if the compounds described herein are obtained as an acid addition salt, the free base can be obtained by basifying a solution of the acid salt. Conversely, if the product is a free base, an addition salt may be produced by dissolving the free base in a suitable organic solvent and treating the solution with an acid, in accordance with conventional procedures for preparing acid addition salts from free base compounds. Those skilled in the art will recognize various synthetic methodologies that may be used to prepare salts. Generally, such salts can be prepared by reacting the free acid or base forms of these compounds with a stoichiometric amount of the appropriate base or acid in water or in an organic solvent, or in a mixture of the two; generally, non-aqueous media like ether (e.g. MTBE), ethyl acetate, alcohols (e.g., methanol, ethanol, iso-propanol or butanol) or acetonitrile (MeCN) are preferred. Lists of suitable salts are found in Remington's Pharmaceutical Sciences, 17^{th} Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selection, and Use, (Wiley, 2002).

Provided also are a pharmaceutically acceptable salt, isotopically enriched analog, deuterated analog, isomer (such as a stereoisomer), tautomer, mixture of isomers (such as a mixture of stereoisomers), and prodrugs of the compounds described herein. “Prodrug” refers to a precursor form of any biologically active compound. A prodrug undergoes a biotransformation (e.g., enzyme cleavage) or chemical transformation (e.g., hydrolysis) before exhibiting pharmacological effects.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

In certain instances the salts of compounds are pharmaceutically acceptable salts. The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from non-toxic inorganic and organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol or butanol) or acetonitrile (MeCN) are preferred. Lists of suitable salts are found in Remington's Pharmaceutical Sciences, 17^{th} Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selection, and Use, (Wiley, 2002).

The term “solvate” refers to a complex formed by combination of solvent molecules with molecules or ions of the solute. The solvent can be an organic compound, an inorganic compound, or a mixture of both. As used herein, the term “solvate” includes a “hydrate” (i.e., a complex formed by combination of water molecules with molecules or ions of the solute), hemi-hydrate, channel hydrate, etc. Some examples of solvents include, but are not limited to, methanol, N,N-dimethylformamide, tetrahydrofuran, dimethylsulfoxide, and water. In general, the solvated forms are equivalent to unsolvated forms and are encompassed within the scope of the present disclosure.

The term “a stereochemically isomeric form” of a compound refers to a stereoisomer of the compound.

The term “tautomer” means compounds produced by the phenomenon wherein a proton of one atom of a molecule shifts to another atom of the molecule. The tautomers also refer to one of two or more structural isomers that exist in equilibrium and are readily converted from one isomeric form to another. Non-limiting examples include enol-keto, imine-enamine, amide-imidic acid tautomers, the tautomeric forms of heteroaryl groups containing a —N═C(H)—NH-ring atom arrangement, such as pyrazoles, imidazoles, benzimidazoles, triazoles, and tetrazoles, and the tautomeric forms of hydroxy substituted 6-membered heteroaryl groups (e.g., hydroxy substituted pyridine, pyrimidine, pyrazine or pyridazine) such as 4-hydroxypyridine and puridin-4(1H)-one, and the like. The compounds described herein may have one or more tautomers and therefore include various isomers. A person of ordinary skill in the art would recognize that other tautomeric ring atom arrangements are possible. All such isomeric forms of these compounds are expressly included in the present disclosure.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

The compounds of the invention, or their pharmaceutically acceptable salts include an asymmetric center and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present invention contemplates various stereoisomers and mixtures thereof and includes “enantiomers,” which refers to two stereoisomers whose molecules are nonsuperimposeable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

Compounds of formula (XXIIIa) are described in U.S. Pat. No. 9,783,538, which is incorporated by reference in its entirety herein. In an exemplary embodiment, a compound of formula (XXIIIa) is 1-{6-[(4-fluorophenyl)methyl]-5-(hydroxymethyl)-3,3-dimethyl-1H,2H,3H-pyrrolo[3,2-b]pyridin-1-yl}-2-[(2R,5R)-5-methyl-2-{[(3R)-3-methylmorpholin-4-yl]methyl}piperazin-1-yl]ethan-1-one referred to herein as the compound of formula (XXII). The compound of formula (XXII)

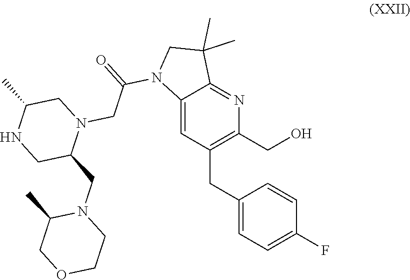 [SMILES: C[C@@H]1CN(CC(=O)N2CC(C)(C)c3nc(CO)c(Cc4ccc(F)cc4)cc32)[C@@H](CN2CCOC[C@H]2C)CN1]

acts as an IAP and cIAP/XIAP antagonist and can be used in various drug formulations to treat various cancers described herein and in U.S. Pat. No. 9,783,538.

In an embodiment, the compound of formula (XXII) is in the form of the L(+)-lactic acid salt of the compound of formula (XXII), acts as an IAP and cIAP/XIAP antagonist, and is used for the treatment of solid tumors and other conditions and diseases. The L(+)-lactic acid salt of the compound of formula (XXII), is referred to herein as the compound of formula (XXIII).

Methods of synthesizing the compounds of formulas (XXIII) and (XXIIIa) are described in U.S. Pat. Nos. 9,783,538; 9,617,248; 9,617,283; 9,663,512; 9,980,973; 9,018,214 and 9,676,768 incorporated by reference in their entirety herein. Synthesis Schemes 1-3 disclosed in U.S. Pat. No. 9,783,538 at Columns 45-50 result in lower yield and purity of the end-product of compounds of formulas (XXIII) and (XXIIIa), relative to the synthesis routes and embodiments of the present application disclosed herein. For example, synthesis Scheme 1 disclosed in U.S. Pat. No. 9,783,538 at Columns 45-46 depicts a general method of preparing a compound of formula (VIIIa). However, synthesis Scheme 1 disclosed in U.S. Pat. No. 9,783,538 at Columns 45-46 results in lower yield and purity of the compounds of formulas (XXIII) and (XXIIIa) relative to the synthesis routes and embodiments of the present application. In addition, Scheme 2 of U.S. Pat. No. 9,783,538 results in low yield, generates a bis-hydroxymethyl impurity in the final product and other impurities in intermediates and end-products that are difficult to purge, such as the bis-hydroxymethyl impurity of formula (XXIV):

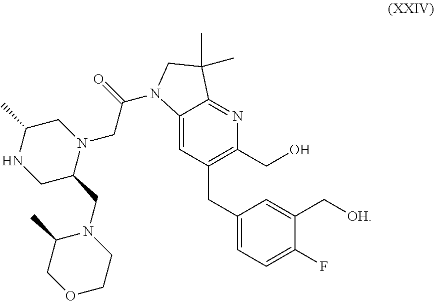 [SMILES: C[C@@H]1CN(CC(=O)N2CC(C)(C)c3nc(CO)c(Cc4ccc(F)c(CO)c4)cc32)[C@@H](CN2CCOC[C@H]2C)CN1]

The synthesis methods of U.S. Pat. No. 9,783,538 also use tert-butyl lithium as a reagent, which limits the scope for large scale manufacture, is a limited supply reagent, is less selective, and highly flammable, volatile, pyrophoric and reactive.

The compounds, intermediates and methods of synthesis of the present application and embodiments disclosed herein are used in improved processes for the preparation of the key intermediate compound of formula (IX) (tert-butyl 5-bromo-6-(4-fluorobenzyl)-3,3-dimethyl-2,3-dihydro-1H-pyrrolo[3,2-b]pyridine-1-carboxylate), resulting in higher purity, stability and yields in the compound of formula (XXIII) and pharmaceutical formulations made from the compound of formula (IX).

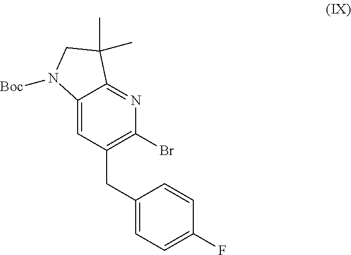 [SMILES: CC(C)(C)OC(=O)N1CC(C)(C)c2nc(Br)c(Cc3ccc(F)cc3)cc21]

The compound of formula (XXIII) prepared according to the synthesis methods and embodiments disclosed herein have enhanced properties, such as less tackiness, higher purity, higher stability and higher overall yield. The purity can be improved by minimizing aldehyde impurities and controlling palladium levels in the end-product. In an exemplary embodiment, the synthesis methods and embodiments for producing the compound of formula (XXIII) disclosed herein yield an end-product of 95% purity or higher, and in another embodiment 98% purity or higher.

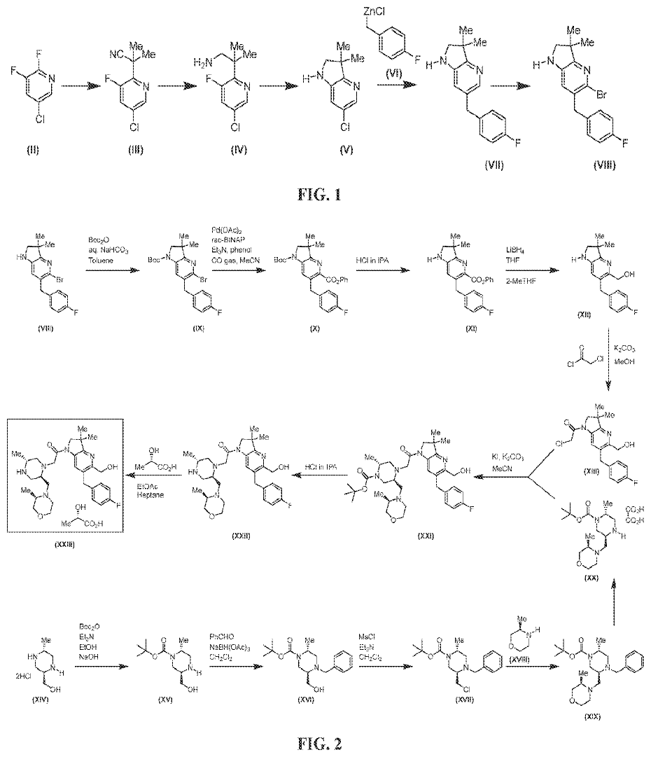

FIG. 1 depicts an exemplary embodiment of the synthesis of compound of formula (VIII) from the compound of formula (II). The compound of formula (VIII) can be used in the synthesis depicted in of FIG. 2 to produce the key intermediate compound of formula (IX).

FIG. 2 depicts an exemplary embodiment of an improved scalable process for the synthesis of the compound of formula (XXIII). The compound of formula (IX) in FIG. 2 is a key intermediate in the production of the compound of formula (XXIII).

The conversion of compound of formula (II) to the compound of formulas (VII) and (VIII) presents serval challenges and deficiencies. For example, the compound of formula (VI) is scarcely available and supplied as a dilute solution. The compound of formula (VI) can take several months to manufacture in quantities necessary for industrial processes, including for use in the production of the compounds of formulas (IX) and (XXIII). The compound of formula (VI) is also highly sensitive to air and moisture, making it difficult, inefficient and unpredictable to use in the synthesis of the compounds of formulas (IX) and (XXIII). The PEPPSI™ catalyst ([1,3-bis(2,6-diisopropylphenyl)imidazol-2-ylidene](3-chloropyridyl)palladium(II) dichloride) typically used in the synthesis of FIG. 1 to convert the compound of formula (V) into the compound of formula (VII) is also expensive. Therefore, it is advantageous to circumvent the use of the compound of formula (VI) and the PEPPSI™ catalyst when synthesizing the compound of formula (IX).

Alternate synthesis paths to produce the intermediate compound of formula (IX) are disclosed herein. Problems of the known processes for the preparation of the key intermediate compound of formula (IX) and end-product compound of formula (XXIII) are addressed by the present application and the embodiments and examples disclosed herein. The exemplary synthesis paths do not require the use of the compound of formula (VI) or the PEPPSI™ catalyst in the synthesis of key intermediates including the compound of formula (IX) and end-product compound of formula (XXIII). The exemplary synthesis paths also utilize new chemical entities, such as the compounds of formula (I) to produce the compound of formula (IX) and end-product compound of formula (XXIII) and/or compounds of formulas (Ia), (I), (IX), (X), (XI), (XVIa) and (XX) to produce the compound of formula (XXIII).

In an embodiment, provided is a process for preparing a compound of formula (XXIII)

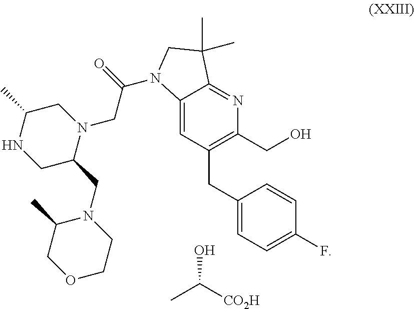

comprising

(i) contacting a compound of formula (XX)

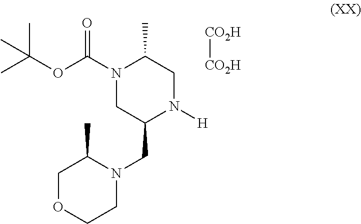

with a compound of formula (XIII)

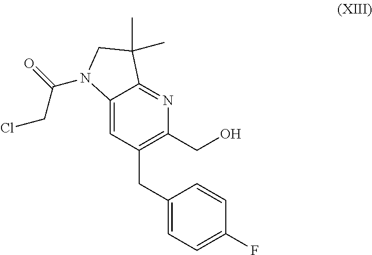 [SMILES: CC1(C)CN(C(=O)CCl)c2cc(Cc3ccc(F)cc3)c(CO)nc21]

under conditions sufficient to provide a compound of formula (XXI), or a salt, solvate or hydrate thereof,

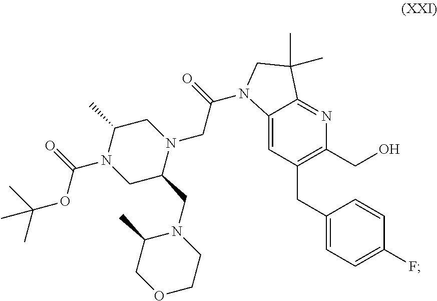 [SMILES: C[C@@H]1COCCN1C[C@H]1CN(C(=O)OC(C)(C)C)[C@H](C)CN1CC(=O)N1CC(C)(C)c2nc(CO)c(Cc3ccc(F)cc3)cc21]

(ii) deprotecting the compound of formula (XXI), or a salt, solvate or hydrate thereof, to provide a compound of formula (XXII), or a salt, solvate or hydrate thereof,

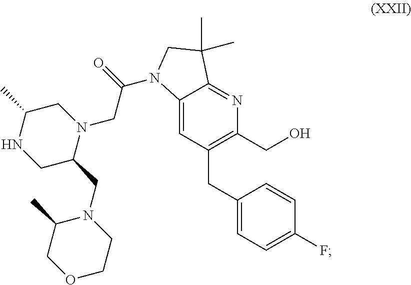 [SMILES: C[C@@H]1CN(CC(=O)N2CC(C)(C)c3nc(CO)c(Cc4ccc(F)cc4)cc32)[C@@H](CN2CCOC[C@H]2C)CN1]

and

(iii) contacting the compound of formula (XXII) with lactic acid to provide the compound of formula (XXIII)

In one embodiment, the lactic acid used in step (iii) above is anhydrous lactic acid described in co-pending application U.S. Provisional Application No. 63/019,87 filed on May 4, 2020 titled “Methods For Synthesizing Anhydrous Lactic Acid,” filed on the same day as the present application and incorporated herein by reference.

In one embodiment, the compound of formula (XIII) is prepared by a process comprising

(i) reacting a compound of formula (V)

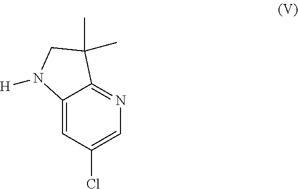

with a compound of formula (VI)

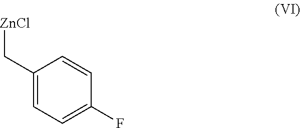

in the presence of one or more palladium catalysts and a ligand to provide a compound of formula (VII)

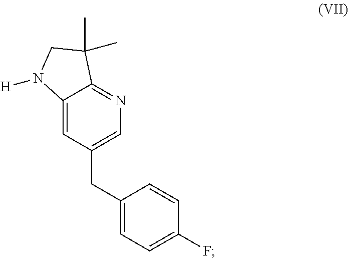 [SMILES: CC1(C)CNc2cc(Cc3ccc(F)cc3)cnc21]

(ii) brominating the compound of formula (VII) to obtain a compound of formula (VIII)

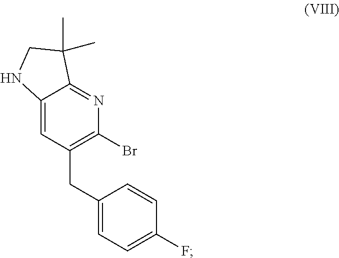 [SMILES: CC1(C)CNc2cc(Cc3ccc(F)cc3)c(Br)nc21]

(iii) protecting the compound of formula (VIII) to provide a compound of formula (IX)

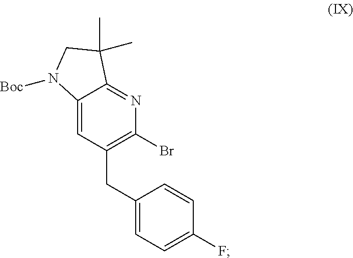 [SMILES: CC(C)(C)OC(=O)N1CC(C)(C)c2nc(Br)c(Cc3ccc(F)cc3)cc21]

(iv) contacting the compound of formula (IX) with carbon monoxide under conditions sufficient to provide the compound of formula (X), a salt, solvate or hydrate thereof.

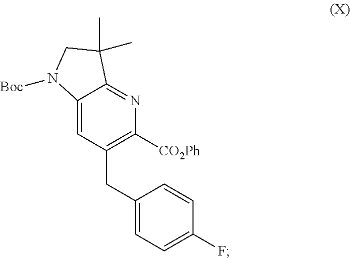

(v) removing the tert-butyloxy carbonyl protecting group from a compound of formula (X), or a salt, solvate or hydrate thereof, to provide a compound of formula (XI)

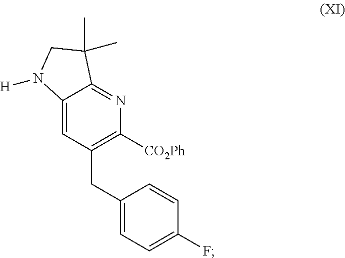

(vi) reducing the compound of formula (XI) to provide a compound of formula (XII)

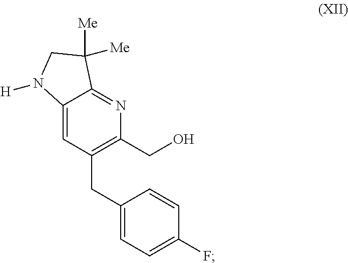 [SMILES: CC1(C)CNc2cc(Cc3ccc(F)cc3)c(CO)nc21]

and

(vii) contacting the compound of formula (XII) with chloroacetyl chloride to provide the compound of formula (XIII).

In one embodiment, provided is a process for preparing a compound of formula (XXIII)

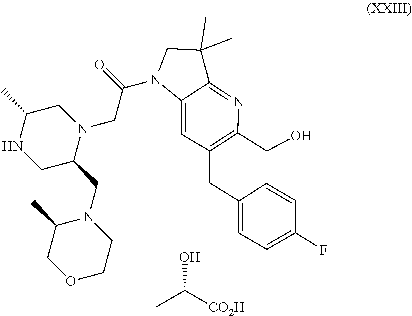

comprising

(i) contacting a compound of formula (IX), or a salt, solvate or hydrate thereof,

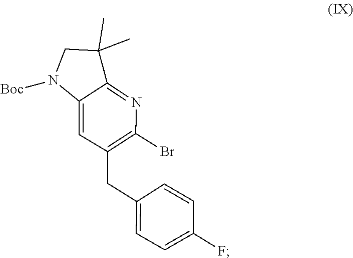

with carbon monoxide under conditions sufficient to provide the compound of formula (X), a salt, solvate or hydrate thereof

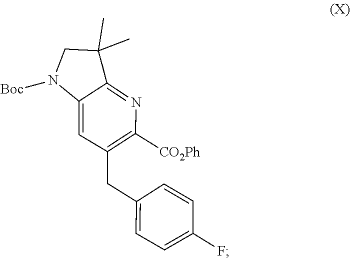

(ii) removing the tert-butyloxy carbonyl protecting group from the compound of formula (X), or a salt, solvate or hydrate thereof, to provide a compound of formula (XI), or a salt, solvate or hydrate thereof,

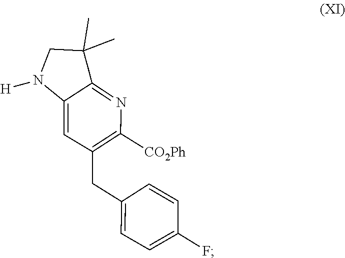

(iii) reducing the compound of formula (XI), or a salt, solvate or hydrate thereof, to provide a compound of formula (XII), or a salt, solvate or hydrate thereof,

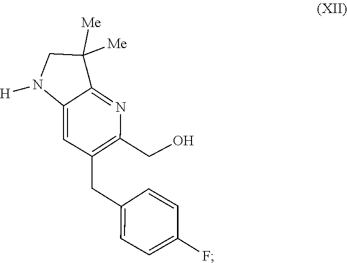

(iv) contacting the compound of formula (XII), or a salt, solvate or hydrate thereof, with chloroacetyl chloride to provide the compound of formula (XIII), or a salt, solvate or hydrate thereof,

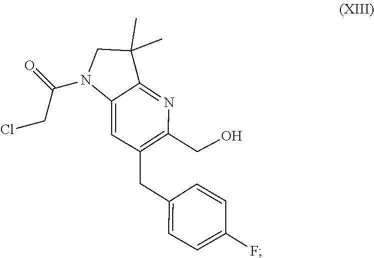

(v) contacting the compound of formula (XIII), or a salt, solvate or hydrate thereof, with a compound of formula (XX)

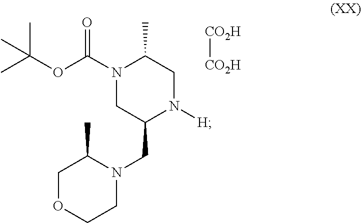

under conditions sufficient to provide a compound of formula (XXI), or a salt, solvate or hydrate thereof,

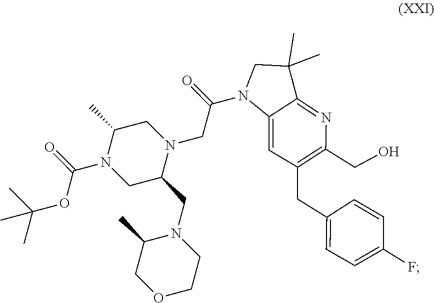

and

(vi) deprotecting the compound of formula (XXI), or a salt, solvate or hydrate thereof, to provide a compound of formula (XXII), or a salt, solvate or hydrate thereof,

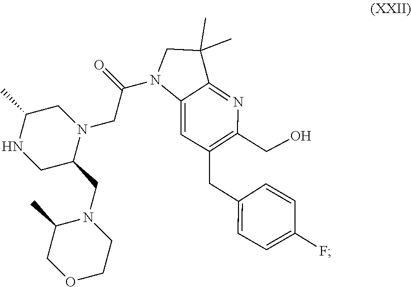

and

(vii) contacting the compound of formula (XXII) with lactic acid to provide the compound of formula (XXIII).

In an embodiment, provided is a process for preparing a compound of formula (XXI), or a salt, solvate or hydrate thereof,

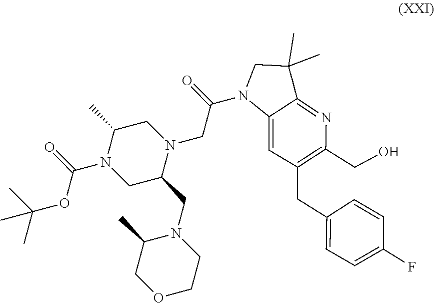

comprising

contacting a compound of formula (XX)

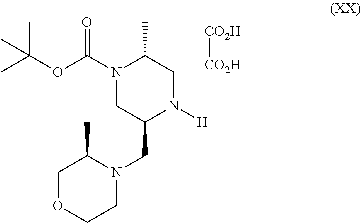

with a compound of formula (XIII)

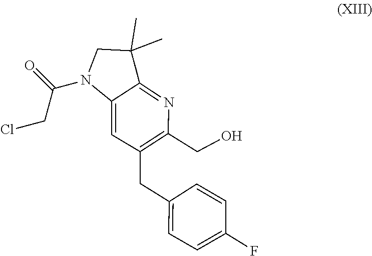

under conditions sufficient to provide a compound of formula (XXI), or a salt, solvate or hydrate thereof.

In an embodiment, provided is a process of preparing a compound of formula (XX)

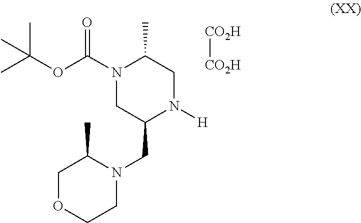

comprising

(i) debenzylating a compound of formula (XIX)

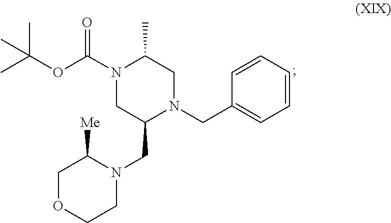 [SMILES: C[C@@H]1COCCN1C[C@H]1CN(C(=O)OC(C)(C)C)[C@H](C)CN1Cc1ccccc1]

and

(ii) contacting the debenzylated product with oxalic acid in a solvent to provide the compound of formula (XX).

In an embodiment of the process for preparing a compound of formula (XX), the debenzylation in step (i) is carried out in the presence of palladium on carbon and hydrogen gas. In an embodiment of the process for preparing a compound of formula (XX), the solvent in step (ii) is ethanol.

In an embodiment, provided is a process for preparing a compound of formula (X), or a salt, solvate or hydrate thereof,

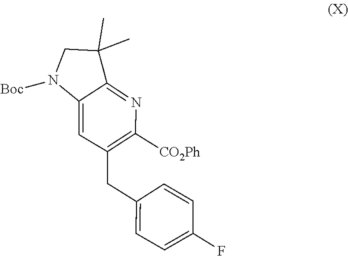

comprising contacting a compound of formula (IX)

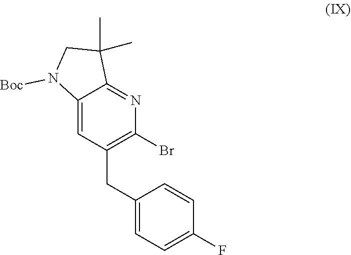 [SMILES: CC(C)(C)OC(=O)N1CC(C)(C)c2nc(Br)c(Cc3ccc(F)cc3)cc21]

with carbon monoxide under conditions sufficient to provide the compound of formula (X), a salt, solvate or hydrate thereof.

In one embodiment of the process for preparing a compound of formula (X), the conditions comprise a palladium catalyst, a ligand, and (i) phenyl formate or phenol, and (ii) carbon monoxide.

In one embodiment of the process for preparing a compound of formula (X), the palladium catalyst is palladium(II) acetate and the ligand is rac-1,1′-binaphthyl-2,2′-diphenyl phosphene.

In one embodiment of the process for preparing a compound of formula (X), the conditions further comprise a base. In an embodiment, the base is triethylamine. Any other suitable base is contemplated within the scope of embodiments presented herein.

In one embodiment of the process for preparing a compound of formula (X), the reaction temperature is in a range of about 45° C. to about 75° C. In one embodiment of the process for preparing a compound of formula (X), the reaction temperature is in a range of about 55° C. to about 65° C. In one embodiment of the process for preparing a compound of formula (X), the reaction solvent is acetonitrile.

In an embodiment, provided is a process for preparing a compound of formula (XI), or a salt, solvate or hydrate thereof,

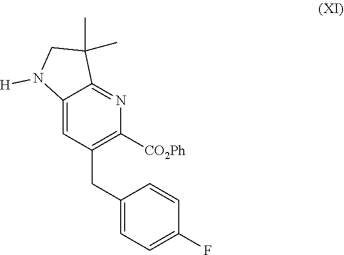

comprising removing the tert-butyoxy carbonyl protecting group from a compound of formula (XI), or a salt, solvate or hydrate thereof,

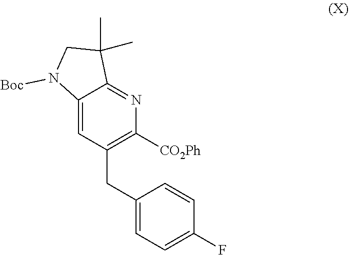

under conditions sufficient to provide the compound of Formula (XI), or a salt, solvate or hydrate thereof.

In an embodiment, the process above further comprises

(i) reducing the compound of formula (XI) under conditions sufficient to provide a compound of formula (XII)

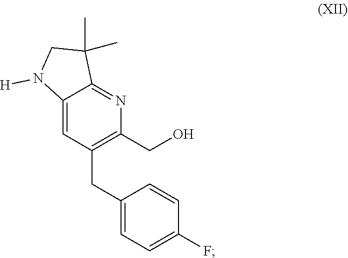 [SMILES: CC1(C)CNc2cc(Cc3ccc(F)cc3)c(CO)nc21]

and

(ii) contacting the compound of formula (XII) with 2-chloroacetyl chloride to provide a compound of formula (XIII)

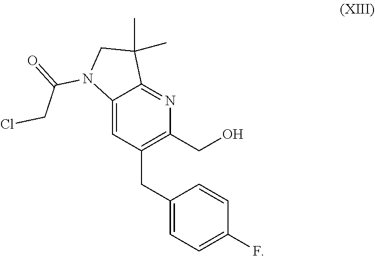 [SMILES: CC1(C)CN(C(=O)CCl)c2cc(Cc3ccc(F)cc3)c(CO)nc21]

In an embodiment, the reducing is conducted in the presence of lithium borohydride. Any other suitable reducing agent (e.g., NaBH₄, LiAlH₄) is contemplated within the scope of embodiments presented herein.

In an embodiment, the solvent for reducing the compound of formula (XI) is 2-methyl tetrahydrofuran.

In an embodiment, the contacting of formula (XII) with 2-chloroacetyl chloride is conducted at a temperature of about of −10° C. to about 0° C.

In an embodiment, provided is a process for preparing a compound of formula (IX), or a salt, solvate or hydrate thereof,

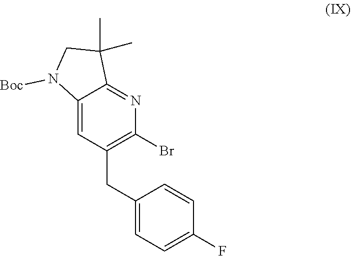 [SMILES: CC(C)(C)OC(=O)N1CC(C)(C)c2nc(Br)c(Cc3ccc(F)cc3)cc21]

comprising

(i) borylating a compound of formula (III), or a salt, solvate or hydrate thereof,

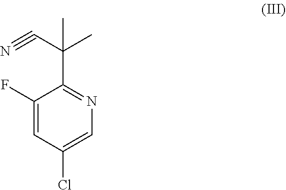

under conditions sufficient to provide a compound of formula (IIIa)

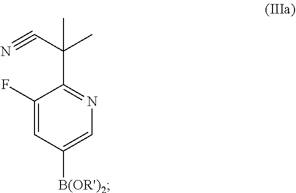

(ii) contacting the compound of formula (IIIa) with 4-fluorobenzyl chloride or 4-fluoro benzyl bromide under conditions sufficient to provide a compound of formula (IIIb), or a salt, solvate or hydrate thereof,

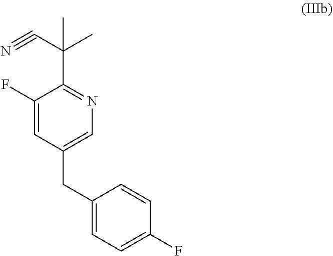 [SMILES: CC(C)(C#N)c1ncc(Cc2ccc(F)cc2)cc1F]

(iii) contacting the compound of formula (IIIb) with a reducing agent to provide a compound of formula (IIIc), or a salt, solvate or hydrate thereof,

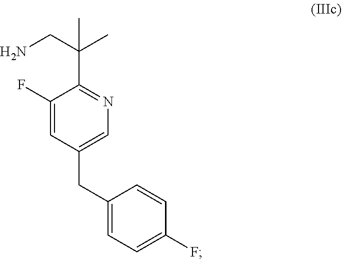 [SMILES: CC(C)(CN)c1ncc(Cc2ccc(F)cc2)cc1F]

(iv) cyclizing the compound of formula (IIIc) to provide a compound of formula (VII), or a salt, solvate or hydrate thereof,

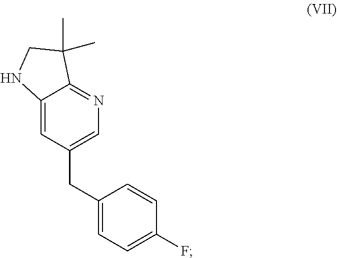 [SMILES: CC1(C)CNc2cc(Cc3ccc(F)cc3)cnc21]

(v) brominating the compound of formula (VII) to provide a compound of formula (VIII), or a salt, solvate or hydrate thereof,

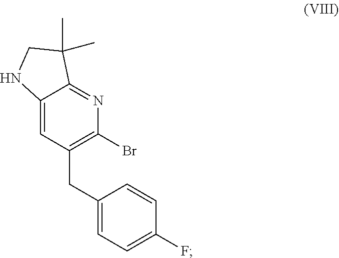 [SMILES: CC1(C)CNc2cc(Cc3ccc(F)cc3)c(Br)nc21]

and

(vi) protecting the compound of formula (VIII) with a tert-butyloxy carbonyl group to provide the compound of formula (IX), or a salt, solvate or hydrate thereof.

It will be understood that in formula (III), the chloro group may be changed to any other suitable group, e.g., bromo, triflate and the like.

In an embodiment, provided is a process for preparing a compound of formula (IX), or a salt, solvate or hydrate thereof,

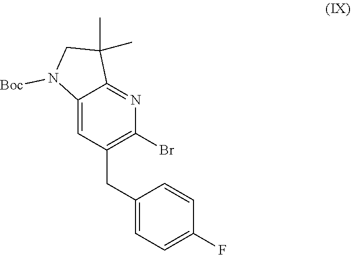 [SMILES: CC(C)(C)OC(=O)N1CC(C)(C)c2nc(Br)c(Cc3ccc(F)cc3)cc21]

comprising

(i) protecting a compound of formula (V), or a salt, solvate or hydrate thereof,

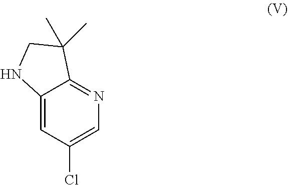

with a tert-butyloxy carbonyl group to provide a compound of formula (Va), or a salt, solvate or hydrate thereof,

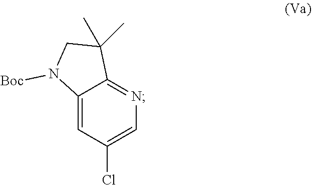 [SMILES: CC(C)(C)OC(=O)N1CC(C)(C)c2ncc(Cl)cc21]

(ii) borylating the compound of formula (Va), or a salt, solvate or hydrate thereof, to obtain a compound of formula (Vb), or a salt, solvate or hydrate thereof,

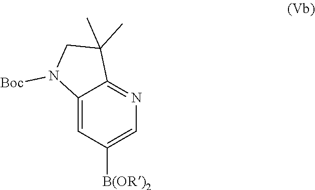

wherein each R′ is independently H, alkyl, or aryl group, or two alkyl or two aryl groups together with the atoms to which they are attached form a dioxaborolanyl ring;

(iii) contacting the compound of formula (Vb), or a salt, solvate or hydrate thereof,

with 4-fluorobenzyl chloride or 4-fluoro benzyl bromide under conditions sufficient to provide a compound of formula (Vc), or a salt, solvate or hydrate thereof,

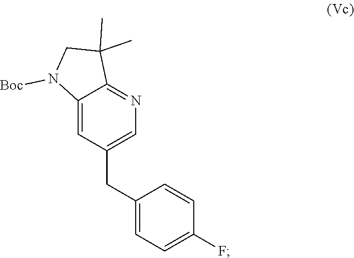 [SMILES: CC(C)(C)OC(=O)N1CC(C)(C)c2ncc(Cc3ccc(F)cc3)cc21]

and

(iv) brominating the compound of formula (Vc) to provide the compound of formula (IX), or a salt, solvate or hydrate thereof.

It will be understood that in formula (V), the chloro group may be changed to any other suitable group, e.g., bromo, triflate and the like.

In an embodiment, provided is a process for preparing a compound of formula (XVIa) comprising contacting a compound of formula (XVI)

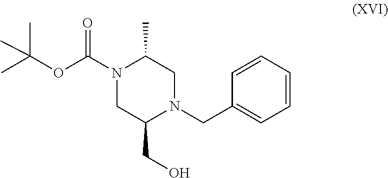 [SMILES: C[C@@H]1CN(Cc2ccccc2)[C@@H](CO)CN1C(=O)OC(C)(C)C]

with oxalic acid in a solvent to provide a compound of formula (XVIa)

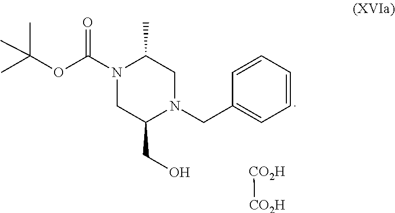 [SMILES: C[C@@H]1CN(Cc2ccccc2)[C@@H](CO)CN1C(=O)OC(C)(C)C ; O=C(O)C(=O)O]

In an embodiment of the process for preparing a compound of formula (VIa), the solvent is methyl tert butyl ether (MTBE).

Provided herein is a compound of formula (XXIII)

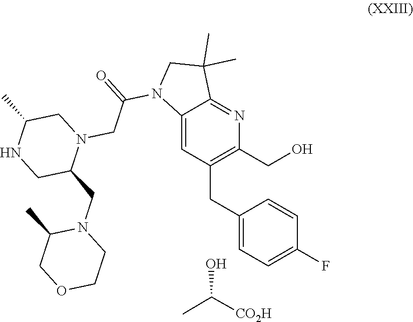

having a purity of at least 95%.

Provided herein is a compound of formula (XXIII)

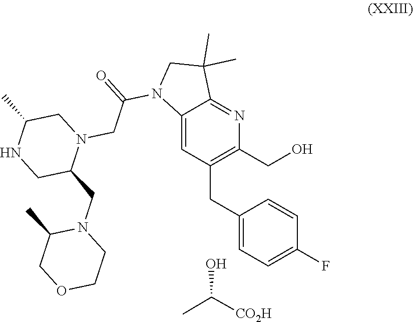

having a purity of at least 98%.

Provided herein is a compound of formula (XXIII), wherein, when the compound of formula (XXIII) is stored at 25° C. and 60% relative humidity for 6 months, the compound of formula (XXIII) comprises no more than about 0.5% a/a of a compound of formula (XXV)

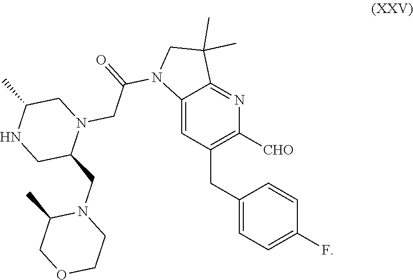 [SMILES: C[C@@H]1CN(CC(=O)N2CC(C)(C)c3nc(C=O)c(Cc4ccc(F)cc4)cc32)[C@@H](CN2CCOC[C@H]2C)CN1]

As used herein, a/a refers to area over area as measured by HPLC. Thus “no more than about 0.5% a/a of a compound of formula (XXV)” means that by HPLC analysis no more than 0.5% of peak areas is attributable to the compound of formula (XXV), or, no more than 1.5% w/w of formula (XXV) is present in the final compound (XXIII).

Provided herein is a compound of formula (XXIII), wherein, when the compound of formula (XXIII) is stored at 25° C. and 60% relative humidity for 6 months, the compound of formula (XXIII) comprises no more than about 0.2% a/a of a compound of formula (XXV).

Provided herein is a compound of formula (XXIII), wherein, when the compound of formula (XXIII) is stored at 25° C. and 60% relative humidity for 12 months, the compound of formula (XXIII) comprises no more than about 0.3% a/a of a compound of formula (XXV).

Provided herein is a compound of formula (XXIII), wherein the compound of formula (XXIII) comprises no more than about 50 ppm of palladium, or no more than about 40 ppm, about 300, or about 20 ppm of palladium.

Provided herein is a composition comprising a compound of formula (XXIII)

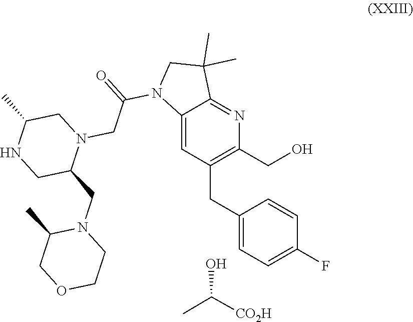

wherein at least 95% of the compound of formula (XXIII) is in Form C.

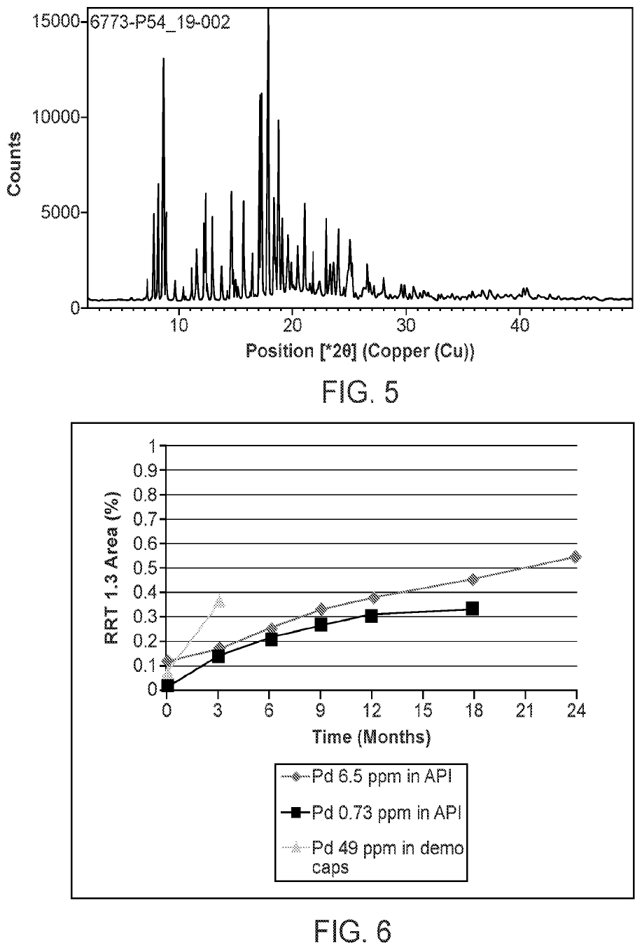

In an embodiment, Form C of formula (XXIII) has an XRPD substantially as shown in FIG. 5.

Provided herein is a compound of formula (Ia), or a salt, solvate or hydrate thereof,

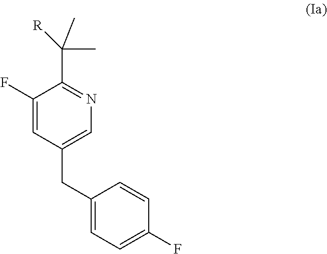

wherein R is CN or CH₂NH₂.

In some embodiments, a compound of formula (Ia) has the structure of formula (IIIb) or (IIIc), or a salt, solvate or hydrate thereof,

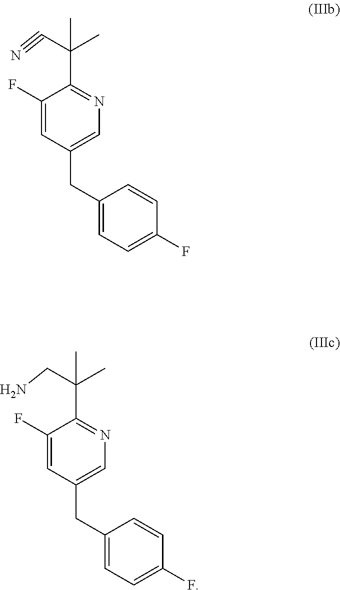 [SMILES: CC(C)(C#N)c1ncc(Cc2ccc(F)cc2)cc1F ; CC(C)(CN)c1ncc(Cc2ccc(F)cc2)cc1F]

Provided herein is a compound of formula (IIIa), or a salt, solvate or hydrate thereof,

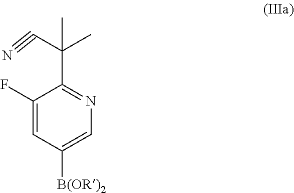

wherein each R′ is independently H, alkyl, or aryl group, or two alkyl or two aryl groups together with the atoms to which they are attached form a dioxaborolanyl ring.

In an embodiment, a compound of formula (IIIa) has the structure of formula (IIIaa)

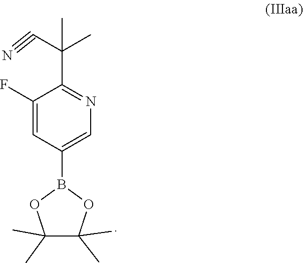 [SMILES: CC(C)(C#N)c1ncc(B2OC(C)(C)C(C)(C)O2)cc1F]

Provided herein is a compound of formula (Vb), or a salt, solvate or hydrate thereof,

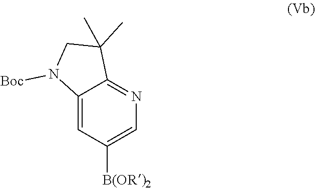

wherein each R′ is independently H, alkyl, or aryl group, or two alkyl or two aryl groups together with the atoms to which they are attached form a dioxaborolanyl ring.

In an embodiment, a compound of formula (Vb) has the structure of formula (Vbb)

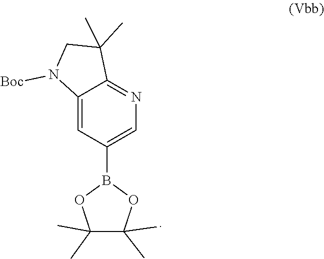 [SMILES: CC(C)(C)OC(=O)N1CC(C)(C)c2ncc(B3OC(C)(C)C(C)(C)O3)cc21]

Provided herein is a compound of formula (I), or a salt, solvate or hydrate thereof,

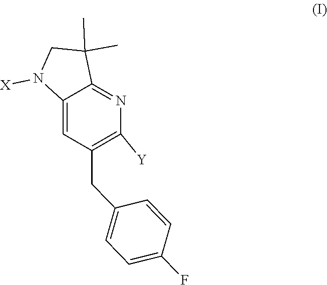

wherein X is H or a protecting group;

Y is COR; and

R is OH, O-alkyl or O-aryl.

Provided herein is a compound of formula (XVIa):

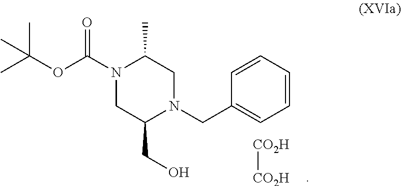

Provided herein is a compound of formula (IX), or a salt, solvate or hydrate thereof,

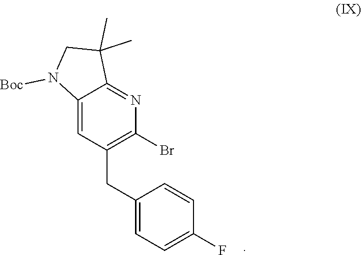 [SMILES: CC(C)(C)OC(=O)N1CC(C)(C)c2nc(Br)c(Cc3ccc(F)cc3)cc21]

Provided herein is a compound of formula (XX):

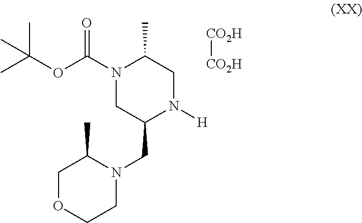

Provided herein is a compound of formula (XI), or a salt, solvate or hydrate thereof,

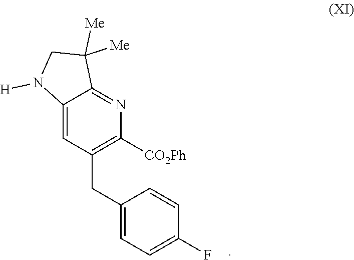

Provided herein is a compound of formula (XXV), or a salt, solvate or hydrate thereof,

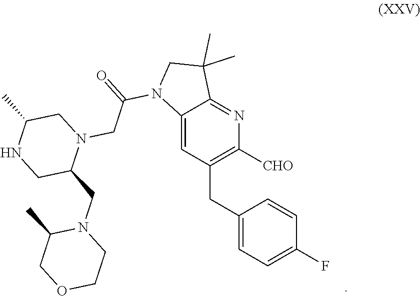 [SMILES: C[C@@H]1CN(CC(=O)N2CC(C)(C)c3nc(C=O)c(Cc4ccc(F)cc4)cc32)[C@@H](CN2CCOC[C@H]2C)CN1]

Provided herein is a compound of formula (IIIa):

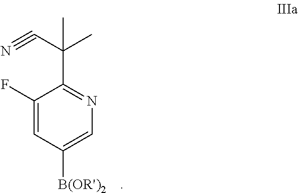

Provided herein is a compound of formula (Vb):

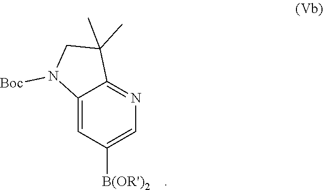

Provided herein is compound (XXIII) prepared by any method described herein.

The compound of formula (VII) can be used in the synthesis of the compound of formula (IX) as shown in synthesis schemes of FIGS. 1 and 2.

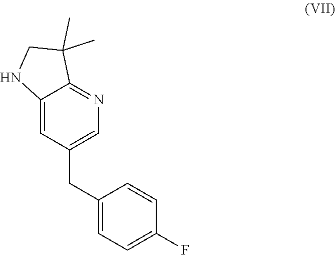 [SMILES: CC1(C)CNc2cc(Cc3ccc(F)cc3)cnc21]

In an exemplary embodiment, a compound of formula (Ia), a salt, solvate or hydrate thereof and methods of synthesizing a compound of formula (Ia), a salt, solvate or hydrate thereof are provided:

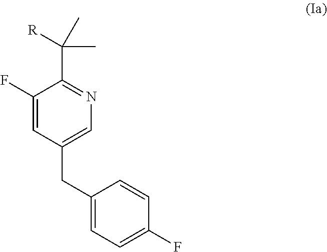

wherein R is CN or CH₂NH₂.

In an exemplary embodiment, a compound of formula (IX), a salt, solvate or hydrate thereof and methods of synthesizing a compound of formula (IX), a salt, solvate or hydrate thereof are provided:

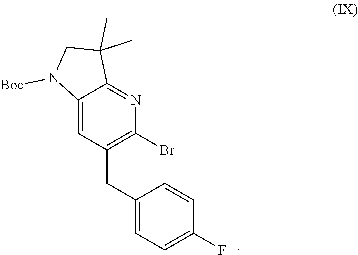 [SMILES: CC(C)(C)OC(=O)N1CC(C)(C)c2nc(Br)c(Cc3ccc(F)cc3)cc21]

In an exemplary embodiment, methods of synthesizing a compound of formula (IX) from compound of formula (Va) are provided:

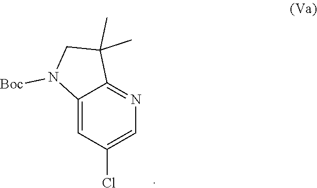 [SMILES: CC(C)(C)OC(=O)N1CC(C)(C)c2ncc(Cl)cc21]

In an exemplary embodiment, methods of synthesizing a compound of formula (VII), a salt, solvate or hydrate thereof are provided:

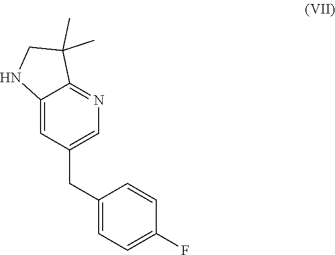 [SMILES: CC1(C)CNc2cc(Cc3ccc(F)cc3)cnc21]

In an exemplary embodiment, methods of synthesizing a compound of formula (VII) from compound of formula (IIIaa) are provided:

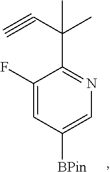

also drawn as

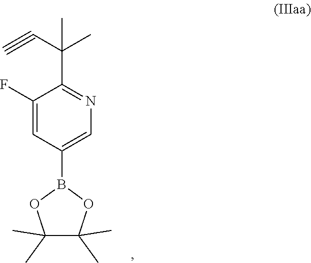 [SMILES: C#CC(C)(C)c1ncc(B2OC(C)(C)C(C)(C)O2)cc1F]

In an exemplary embodiment, a compound of formula (I) and methods of synthesizing a compound of formula (I) are provided:

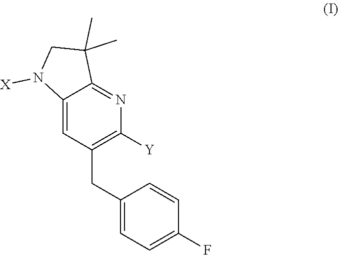

wherein X is H or a protecting group;

### Y is Br, Cl, I or COR; and

### R is H, OH, O-alkyl or O-aryl.

In an exemplary embodiment, a compound of formula (XVI), a salt, solvate or hydrate thereof and methods of synthesizing a compound of formula (XVI), a salt, solvate or hydrate thereof are provided:

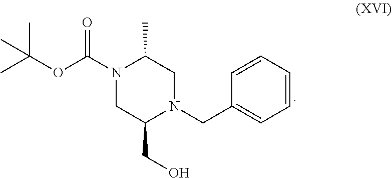 [SMILES: C[C@@H]1CN(Cc2ccccc2)[C@@H](CO)CN1C(=O)OC(C)(C)C]

In an exemplary embodiment, a compound of formula (XXa), a solvate or hydrate thereof and a salt compound of formula (XX) and methods of synthesizing a compound of (XXa), a solvate or hydrate thereof and a salt compound of formula (XX) are provided:

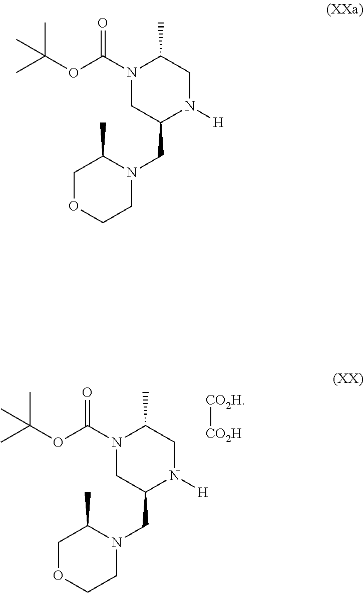

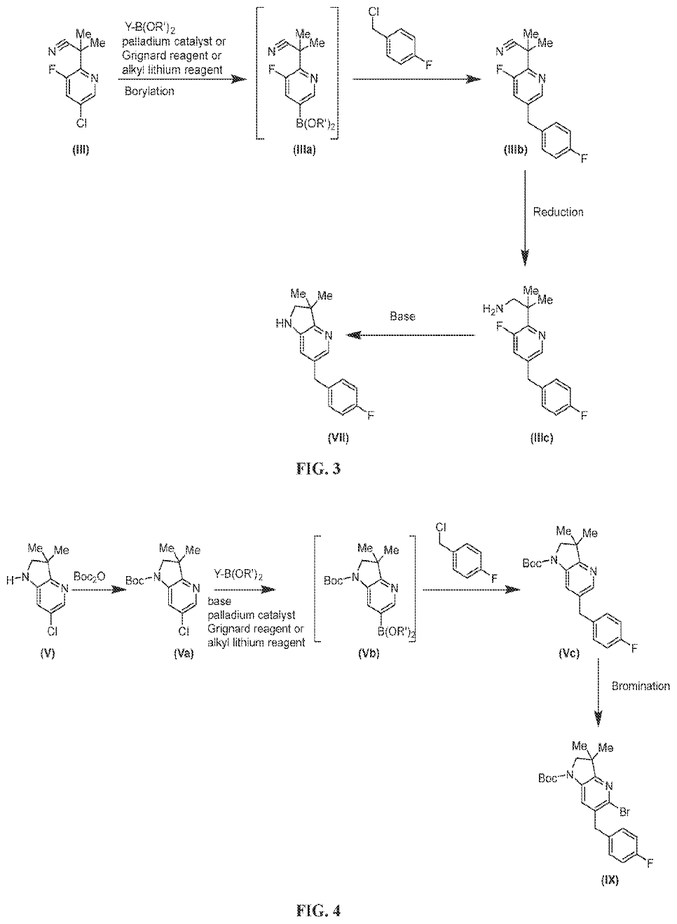

In an exemplary synthesis shown in FIG. 3, the compound of formula (VII) is produced.

In the synthesis of the compound of formula (VII) in FIG. 3, Y is OR′ or B(OR′)₂ and each R′ is independently an H, alkyl, or aryl group or two alkyl or aryl groups forming a ring with B.

Referring to FIG. 3, the borylation of compound of formula (III) produces a boronic ester compound of formula (IIIa). This step is described in Example 1.

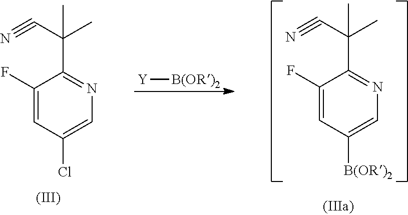

Borylation can be carried out by reacting the compound of formula (III) with a borylation agent and a catalyst. In an exemplary embodiment, the borylation agent can be a compound of formula Y—B(OR′)₂. For the compound of formula (IIIa) and the borylation agent of formula Y—B(OR′)₂, Y is OR′ or B(OR′)₂, and each R′ is independently an H, alkyl, or aryl group or two alkyl or aryl groups forming a ring with B. In an exemplary embodiment the borylation agent is bis(pinacolato)diboron and the catalyst is a palladium catalyst. In one embodiment, a compound of formula (IIIa) is a compound of formula (IIIaa):

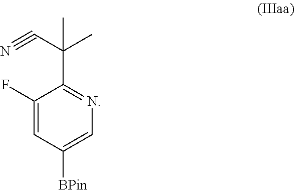

In an exemplary embodiment, the catalyst is one or more palladium catalysts, including but not limited to, a Xphos-Pd-G2 catalyst, Pd(Oac)₂ or PPh₂(dba)₃ with ligands such as PPh₃, Xantphos, DPPE, DPPP, DPPB, DPPF, rac-BINAP, RuPhos or tBuXphos. In an exemplary embodiment, the palladium catalyzed reaction can occur in the presence of a ligand. Suitable ligands include 2-dicyclohexylphosphino-2′,4′,6′-triisopropylbiphenyl (Xphos) used as a precursor to Suzuki coupling.

In another exemplary embodiment, the borylation agent is the compound of formula Y—B(OR′)₂, Y is OR′, and the compound of formula (III) is reacted with the borylating agent in the presence of a Grignard reagent or an alkyl lithium reagent.

The borylation of the compound of formula (III) to produce the compound of formula (IIIa) can occur in the presence of one or more bases, such as potassium acetate, sodium acetate, triethyl amine, diisopropyl ethyl amine, pyridine in one or more organic solvents, such as 2-methyltetrahydrofuran (2-MeTHF), THF, dioxane, toluene, xylene or MTBE.

In an exemplary step of the synthesis of FIG. 3, the compound of formula (IIIa) can be benzylated to produce the compound of formula (IIIb). This step is described in Example 1.

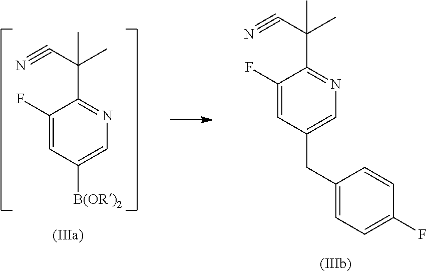

In an exemplary embodiment, the compound of formula (IIIa) can be benzylated with a benzylating agent. For the compound of formula (IIIa), each R′ is independently an H, alkyl, or aryl group or two alkyl or aryl groups forming a ring with B. In an exemplary embodiment, the benzylating agent is a benzyl chloride derivative. Suitable benzyl chloride derivatives include, but are not limited to, 4-fluorobenzyl chloride and 4-fluoro benzyl bromide. The benzylating agent can be used in a Suzuki cross-coupling reaction to produce the compound of formula (IIIb).

The benzylating agents used in this step of the synthesis are stable in air and in the presence of moisture. They are also readily available in industrial quantities and cheaper than the compound of formula (VI). The use of the exemplary benzylating agents in this step increases the stability, predictability and efficiency of the synthesis of the compounds of formulas (IX) and (XXIII).

In an exemplary embodiment, the compound of formula (IIIa) can be benzylated in the presence of a base. Suitable bases include, but are not limited to, potassium carbonate, sodium carbonate, sodium hydroxide, potassium hydroxide and potassium phosphate.

In an exemplary step of the synthesis of FIG. 3, the compound of formula (IIIb) can be reduced to form the compound of formula (IIIc). This step is described in Example 2.

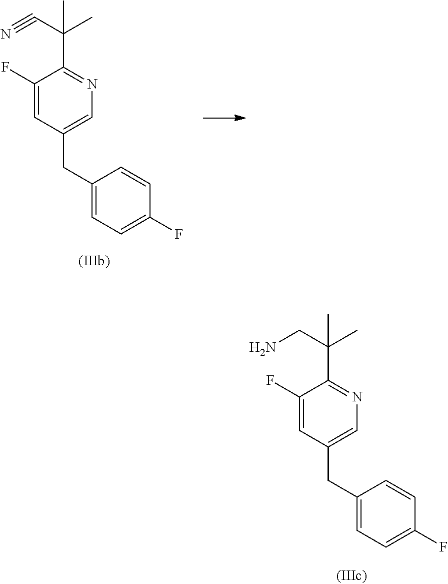 [SMILES: CC(C)(C#N)c1ncc(Cc2ccc(F)cc2)cc1F ; CC(C)(CN)c1ncc(Cc2ccc(F)cc2)cc1F]

The compound of formula (IIIb) can be reduced with a reducing agent. Suitable reducing agents include, but are not limited to, sodium borohydride and lithium aluminum hydride.

In an exemplary embodiment, reduction occurs with a reducing agent in the presence of a nickel catalyst. Suitable nickel catalysts include, but are not limited to, nickel chloride, nickel(II) chloride hexahydrate. In an exemplary embodiment, the compound of formula (IIIb) is reduced by hydrogenation over a Raney nickel catalyst.

In an exemplary step of the synthesis of FIG. 3, the compound of formula (IIIc) is converted to the compound of formula (VII) in a cyclization reaction. This step is described in Example 3.

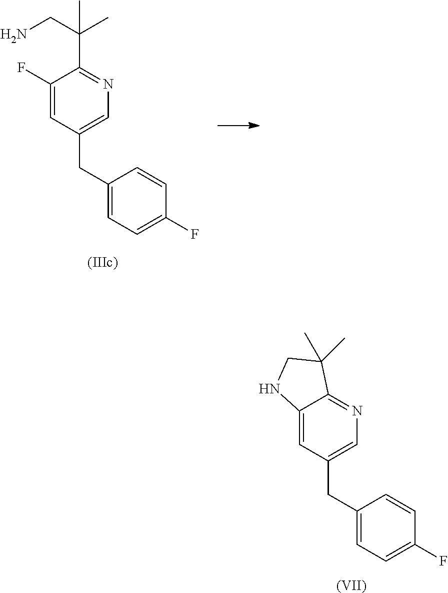 [SMILES: CC(C)(CN)c1ncc(Cc2ccc(F)cc2)cc1F ; CC1(C)CNc2cc(Cc3ccc(F)cc3)cnc21 ; "(VII)"]

The cyclization reaction is carried out by deprotonating the amino group and displacement of the fluorine atom on the pyridine ring in the presence of a base. In an exemplary embodiment, sodium bicarbonate acts as a deprotonating agent, which triggers the dehalogenation and intramolecular nucleophilic displacement, causing cyclization or ring closing. In order to facilitate deprotonation and dehalogenation, the compound of formula (IIIc) can be reacted with a base in the presence of a solvent. Suitable solvents include, but are not limited to, polar aprotic solvents or dimethyl sulfoxide (DMSO), THF, DMF and DMAc. Suitable bases include, but are not limited, to NaHCO₃, NaH, Na₂CO₃ and K₂CO₃.

The compound of formula (VII) can be used in the synthesis of the compound of formula (IX) as shown in FIGS. 1-2 without utilizing the compound of the compound of formula (VI) or the PEPPSI™ catalyst, which cause inefficiencies and unpredictability in the synthesis.

In an alternate embodiment, the compound of formula (VII) is prepared as shown in FIG. 1 and Example 3A. FIG. 1, and Example 3B, describe an exemplary embodiment of the synthesis of the compound of formula (VIII).

Starting with the compound of formula (V) in FIG. 1, the compound of formula (VII) is prepared by reacting the compound of formula (V) with the compound of formula (VI). This step is described in Example 3A.

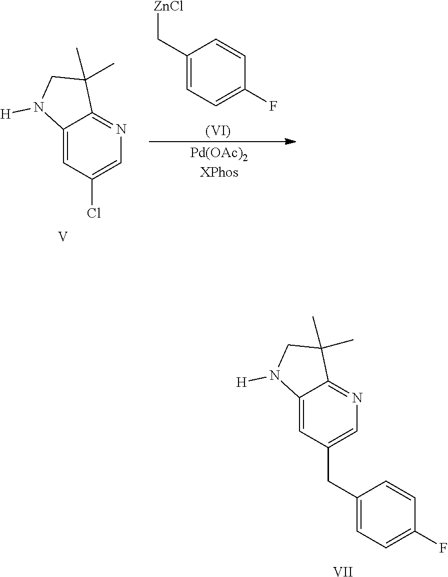

In an exemplary embodiment, the compound of formula (V) can be reacted with the compound of formula (VI) in the presence of one or more palladium catalysts, including but not limited to, a Xphos-Pd-G2 catalyst, Pd(Oac)₂ or Pd₂(dba)₃ with ligands such as PPh₃, Xantphos, DPPE, DPPP, DPPB, DPPF, rac-BINAP, RuPhos or tBuXphos. In an exemplary embodiment, the palladium catalyzed reaction can occur in the presence of the ligand 2-dicyclohexylphosphino-2′,4′,6′-triisopropylbiphenyl (Xphos) used as a precursor to Suzuki coupling. The reaction can also occur in a suitable solvent, such as N-methylpyrrolidinone (NMP) and/or tetrahydrofuran (TIF). Applicant has found that this step of the synthesis can be performed without the use of the PEPPSI catalyst, which causes inefficiencies and unpredictability in the synthesis.

The compound of formula (VIII) in FIG. 1 can be produced by brominating the compound of formula (VII) with a brominating agent. This step is described in Example 3B.

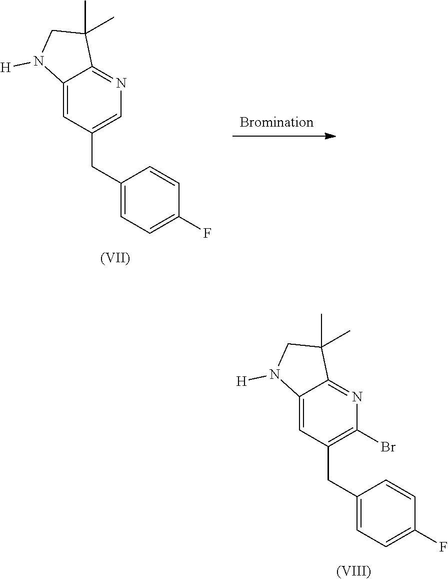 [SMILES: CC1(C)CNc2cc(Cc3ccc(F)cc3)cnc21 ; "(VII)" ; CC1(C)CNc2cc(Cc3ccc(F)cc3)c(Br)nc21 ; "(VIII)"]

In an exemplary embodiment, the compound of formula (VII) is brominated in the presence of a solvent to provide a compound of formula (VIII). Suitable brominating agents include N-bromosuccinimide and dibromo dimethyl hydantoin and suitable solvents include dimethyl formamide. The compound of formula (VIII) can then be used to produce the compound of formula (XIII) for coupling reaction with the compound of formula (XX) to produce the compound of formula (XXI) as shown in the synthesis of FIG. 2.

The compound of formula (IX) in FIG. 2 is prepared from the compound of formula (VIII) by a Boc protection step. This step is described in Example 6B.

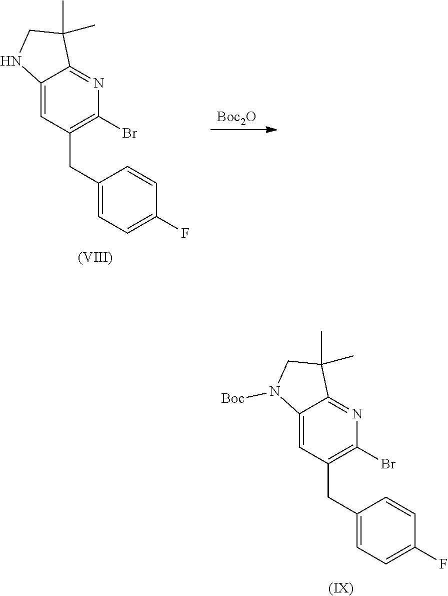

The Boc protection can be achieved by reacting the compound of formula (VIII) with a Boc protecting group, such as di-tert-butyl dicarbonate. In an example embodiment, the Boc protection reaction can occur in the presence of one or more reagents including a base and a solvent. Suitable bases include, but are not limited to, sodium carbonate, N,N-dimethylamino pyridine, sodium hydroxide, triethylamine, sodium bicarbonate, potassium carbonate and diisopropylethylamine. Suitable solvents include, but are not limited to, toluene, dichloromethane, ethyl acetate and water as an optional co-solvent.

Other suitable protection groups include carboxybenzyl (Cbz) groups.

Alternatively, the exemplary synthesis shown in FIG. 4 also produces the compound of formula (IX).

In the synthesis of the compound of formula (IX) in FIG. 4, Y is OR′ or B(OR′)₂ and each R′ is independently an H, alkyl, or aryl group or two alkyl or aryl groups forming a ring with B.

In an exemplary step of the synthesis of FIG. 4, the compound of formula (V) can be converted to the compound of formula (Va) by attaching a protecting group. This step is described in Example 4.

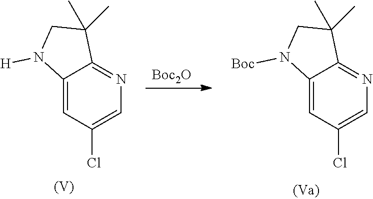

In an exemplary embodiment, the protecting group is a Boc protecting group, and the compound of formula (Va) is prepared by reaction with di-tert-butyl decarbonate, which can be removed with deprotection agents. Protection can occur in the presence of a solvent. Suitable solvents for protection include, but are not limited to, toluene, dichloromethane, THE and acetonitrile. Other protecting groups are contemplated within the scope of embodiments presented herein including and not limited to benzyl, acetyl, and/or carboxybenzyl group (CBz) protecting groups.

In an exemplary step of the synthesis of FIG. 4, the compound of formula (Va) can be converted to the compound of formula (Vb) by borylation. This step is described in Example 5.

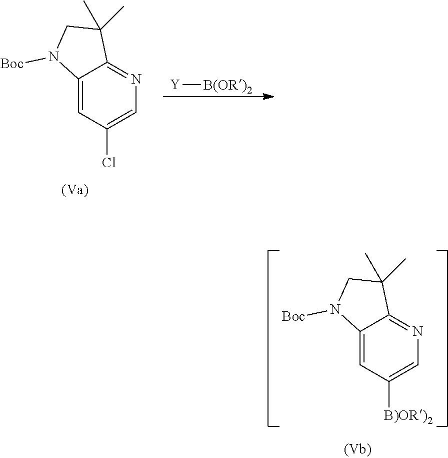

Borylation can be carried out by reacting the compound of formula (Va) with a borylation agent and a catalyst. The borylation agent can be a compound of formula Y—B(OR′)₂. For the compound of formula (Vb) and the borylation agent of formula Y—B(OR′)₂, Y is OR′ or B(OR′)₂, and each R′ is independently an H, alkyl, or aryl group or two alkyl or aryl groups forming a ring with B. In an exemplary embodiment, the borylation agent is bis(pinacolato)diboron and the catalyst is a palladium catalyst.

In an exemplary embodiment, the catalyst is one or more palladium catalysts, including but not limited to, a Xphos-Pd-G2 catalyst, Pd(Oac)₂ or Pd₂(dba)₃ with ligands, such as PPh₃, Xantphos, DPPE, DPPP, DPPB, DPPF, rac-BINAP, RuPhos or tBuXphos. The palladium catalyzed reaction can occur in the presence of a ligand. In an exemplary embodiment, the ligand is 2-dicyclohexylphosphino-2′,4′,6′-triisopropylbiphenyl (Xphos) used as a precursor to Suzuki coupling.

In another exemplary embodiment, the borylation agent is the compound of formula Y—B(OR′)₂, Y is OR′, and the compound of formula (Va) is reacted with the borylating agent in the presence of a Grignard reagent or an alkyl lithium reagent.

The borylation of the compound of formula (Va) to produce the compound of formula (Vb) can also occur in the presence of one or more bases, such as potassium acetate, sodium acetate, triethyl amine, diisopropyl ethyl amine, pyridine in one or more organic solvents, such as 2-methyltetrahydrofuran (2-MeTHF), THF, dioxane, toluene, xylene or MTBE.

In an exemplary step of the synthesis of FIG. 4, the compound of formula (Vb) can be benzylated to produce the compound of formula (Vc). This step is described in Example 5.

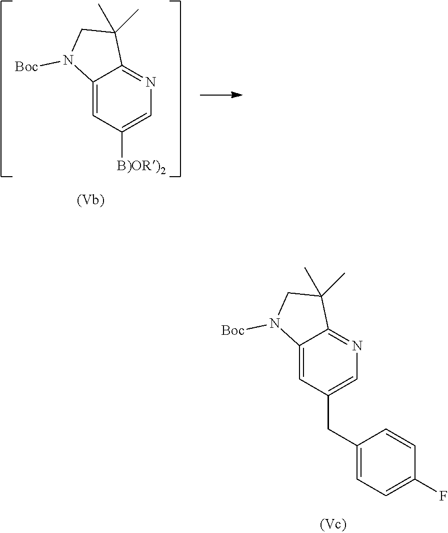

The compound of formula (Vb) can be benzylated with a benzylating agent. As used herein, “benzylated with a benzylating agent” refers to the coupling of the boronate (Vb) with a compound of formula

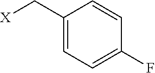

wherein X is a suitable leaving group (e.g., halo, tosyl, triflate and the like). For the compound of formula (Vb), each R′ is independently an H, alkyl, or aryl group or two alkyl or aryl groups forming a ring with B. In an exemplary embodiment, the benzylating agent is a benzyl chloride derivative. Suitable benzylating agents include, but are not limited to, 4-fluorobenzyl chloride and 4-fluoro benzyl bromide. The benzylating agent can be used in a Suzuki cross-coupling reaction to produce the compound of formula (Vc).

The benzylating agents used in this step of the synthesis are stable in air and in the presence of moisture. They are also readily available in industrial quantities and cheaper than the compound of formula (VI). The use of the exemplary benzylating agents in this step increases the stability, predictability and efficiency of the synthesis of the compounds of formulas (IX) and (XXIII).

The compound of formula (Vb) can be benzylated in the presence of a base. Suitable bases include, but are not limited to, potassium carbonate, sodium carbonate, sodium hydroxide, potassium hydroxide and potassium phosphate.

In an exemplary step of the synthesis of FIG. 4, the compound of formula (Vc) can be brominated to produce the compound of formula (IX). This step is described in Example 6.

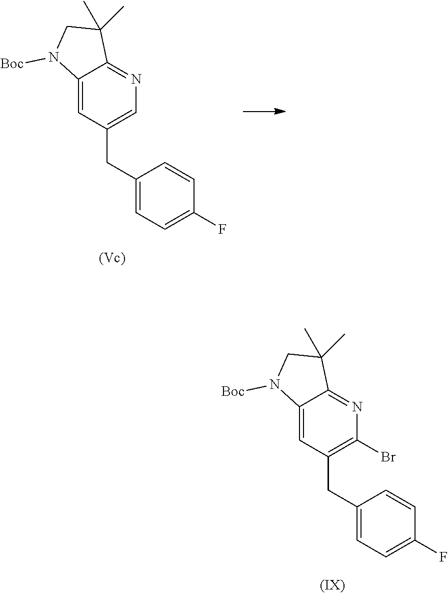

Bromination can be carried out by reacting the compound of formula (Vc) with a brominating agent. Suitable brominating agents, include but are not limited to, 1-bromopyrrolidine-2,5-dione (BMS) and 1,3-Dibromo-5,5-Dimethylhydantoin (DBDMH). Bromination can be carried out in the presence of an organic solvent. Suitable organic solvents include, but are not limited to, dimethylformamide (DMF), dichloromethane, acetonitrile and ethyl acetate.

The synthesis of FIG. 4 can be used to produce the compound of formula (IX) without utilizing the compound of formula (VI) or the PEPPSI™ catalyst, which cause inefficiencies and unpredictability in the synthesis.

The compound of formula (IX) is a key intermediate in the synthesis of the compound of formula (XXIII) and active pharmaceutical ingredients manufactured from the compound of formula (XXIII). The synthesis of the compound of formula (XXIII) with the use of the compound of formula (IX) is described below.

The compound of formula (IX) in FIG. 2 can be converted to the compound of formula (X) by reaction with phenol in a palladium catalyzed carbonylation to form the phenyl ester compound of formula (X). This step is described in Example 7.

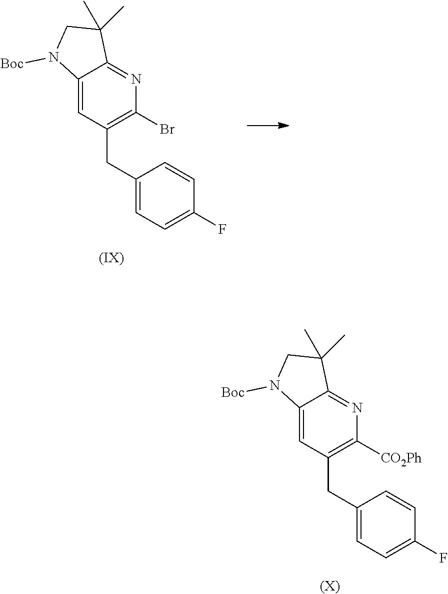

The palladium catalyzed carbonylation of the compound of formula (IX) can be achieved by reacting the compound of formula (IX) with phenol and carbon monoxide in the presence of a palladium catalyst. Suitable palladium catalysts can include Xphos-Pd-G2 catalyst, Pd(Oac)₂ or Pd₂(dba)₃ with ligands, such as PPh₃, Xantphos, DPPE, DPPP, DPPB, DPPF, rac-BINAP, RuPhos or tBuXphos. In an exemplary embodiment, the palladium catalyst is palladium(II) acetate and the ligand is rac-BINAP. Alternatively, carbonylation of the compound of formula (IX) can be achieved by reacting the compound of formula (IX) with phenyl formate in the presence of a palladium catalyst.

The compound of formula (X) in FIG. 2 can be converted to the compound of formula (XI) by deprotection or removal of the Boc group. This step is described in Example 8.

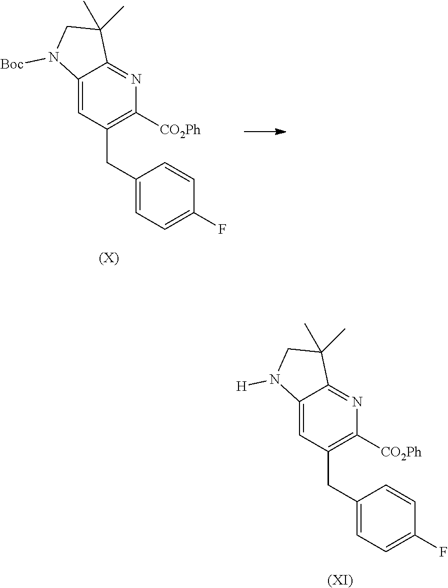

In an exemplary embodiment, the Boc protecting group can be removed with a deprotection agent. Suitable deprotection agents include, but are not limited to, HCl, TFA, HBr, MsOH, TsOH, CSA or other acids. In an exemplary embodiment, deprotection can occur in the presence of a solvent. Suitable solvents include but are not limited to isopropyl alcohol, methanol, ethanol, t-butanol, THE or MeCN.

The compound of formula (XI) in FIG. 2 can be converted to the compound of formula (XII) by phenyl ester reduction to form the alcohol compound of formula (XII). This step is described in Example 9.

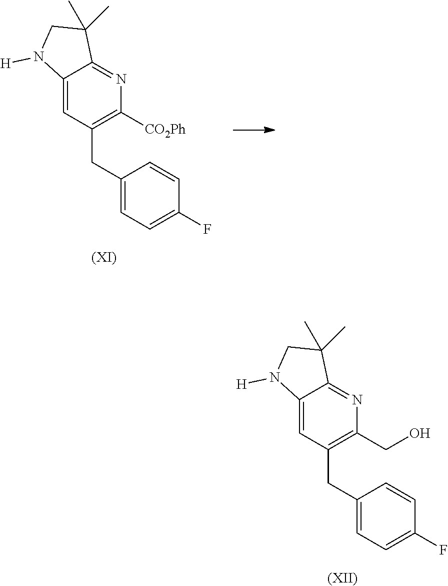

Suitable reducing agents to reduce the phenyl ester into an alcohol include, but are not limited to, lithium borohydride, sodium borohydride, lithium aluminum hydride, borane, sodium triacetoxy borohydride, L-selectride, K-selectride, Red-Al and DIBAL-H. Reduction can occur in the presence of a solvent, such as THE compounds (e.g., methyl tetrahydrofuran).

The compound of formula (XII) in FIG. 2 can be converted to the compound of formula (XIII) by chloroacetylation of the amino group. This step is described in Example 10.

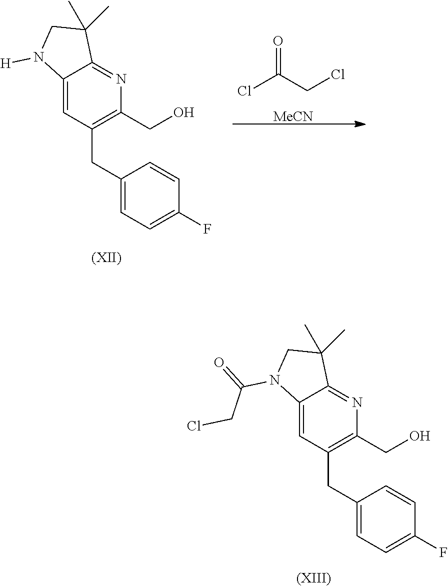

The chloroacetylation can be achieved with 2-chloroacetyl chloride, acetonitrile and solvent, such as dichloromethane, tetrahydrofuran and/or toluene.

Referring to FIG. 2, the compound of formula (XX) is prepared or synthesized from the compound of formula (XIV).

Boc protection of the compound of formula (XIV) yields a diBoc intermediate, which is not isolated. Treatment of the diBoc intermediate with a suitable base produces the compound of formula (XV). This step is described in Example 11.

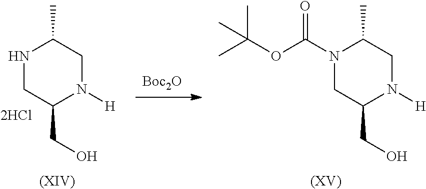

In an exemplary embodiment, Boc protection can be carried out by reacting the compound of formula (XIV) with a tert-butyloxycarbonyl (Boc) protecting group. The reaction can occur in the presence of one or more reagents, including one or more bases and/or solvents. Suitable bases include, but are not limited, to sodium hydroxide, potassium hydroxide, sodium carbonate, N,N-dimethylamino pyridine and triethylamine. Suitable solvents include, but are not limited to, toluene, methanol, dichloromethane, ethyl acetate and ethanol.

The compound of formula (XV) in FIG. 2 can be converted to the compound of formula (XVI) by benzyl group protection. This step is described in Example 12.

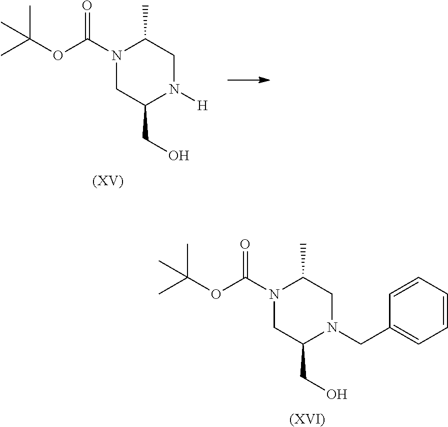

In an exemplary embodiment, benzyl group protection is carried out with a benzylating agent. A suitable benzylating agent is benzaldehyde. Benzylation can occur in the presence of one or more solvents and reducing agents. Suitable solvents include, but are not limited to, dichloromethane, ethyl acetate and ethanol. Suitable reducing agents include, but are not limited to, sodium triacetoxyborohydride, sodium borohydride (NaBH₄), borane and diisobutylaluminium hydride (DIBAL-H).

Alternatively, the compound of formula (XV) can be converted and isolated into the oxalate salt compound of formula (XVIa) by reaction with oxalic acid in suitable solvent, such as methyl tert-butyl ether. This step is described in Example 13. The isolated oxalate salt compound of formula (XVIa) can be used in the next step of the synthesis of FIG. 2 to provide a higher purity end-product.

Chlorination of the compound of formula (XVI) yields the chloro-compound of formula (XVII). This step is described in Example 14.

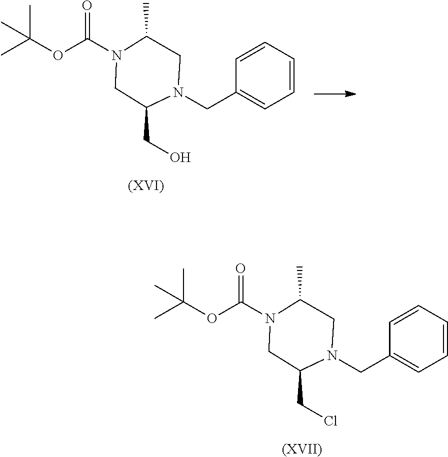

Chlorination can be achieved by reacting the compound of formula (XVI) with a chlorinating agent. Suitable chlorinating agents include, but are not limited to, methanesulfonyl chloride, thionyl chloride, sulfuryl chloride, phosphoryl chloride (POCl₃) and phosphorus trichloride (PCl₃). In an exemplary embodiment, chlorination can be achieved in the presence of one or more bases and/or solvents. A suitable base includes triethylamine and suitable solvents include, but are not limited to, dichloromethane, ethyl acetate and ethanol.

Nucleophilic displacement of the compound of formula (XVII) yields the compound of formula (XIX). This step is described in Example 15.

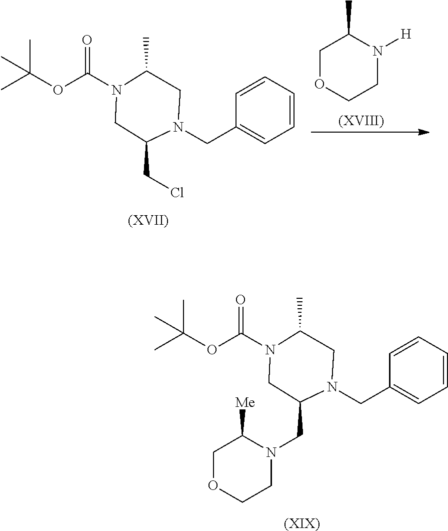

Nucleophilic displacement can be achieved by reacting the compound of formula (XVII) with a nucleophile. In an exemplary embodiment, the nucleophile is the compound of formula (XVIII) (3-methyl morpholine, hydrochloride salt). Nucleophilic displacement can be carried out with a nucleophile in the presence of a solvent and a base. Suitable solvents include acetonitrile and suitable bases include, but are not limited to, potassium carbonate, sodium carbonate and potassium phosphate. Additional additives can be used to promote the reaction, such as potassium iodide.

Debenzylation of the compound of formula (XIX) yields the compound of formula (XX). This step is described in Example 16.

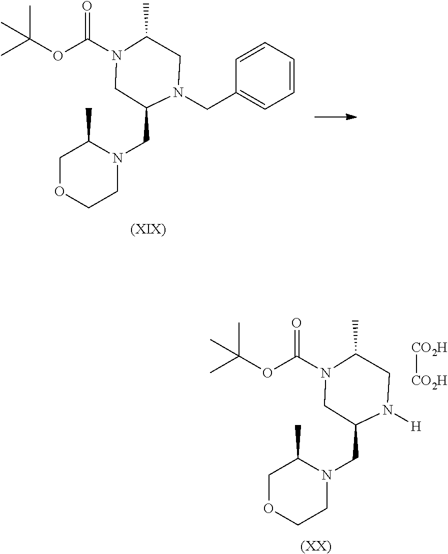

Debenzylation can be achieved by reaction of the compound of formula (XIX) with hydrogen and one or more palladium catalysts. Debenzylation can occur in the presence of a solvent, such as ethanol, methanol, toluene and heptane. In an exemplary embodiment, the solvent is anhydrous ethanol. Suitable palladium catalysts include palladium on activated carbon and palladium hydroxide. Treatment with oxalic acid produces the oxalate salt compound of formula (XX). Using the oxalate salt compound of formula (XX) reduces impurities in the final end-product compound of formula (XXIII).

The high purity end-product of formula (XXIII) can then be produced by a coupling reaction with two additional keys steps. Coupling between the compound of formula (XIII) and the compound of formula (XX) produces the compound of formula (XXI) as described in Example 17.

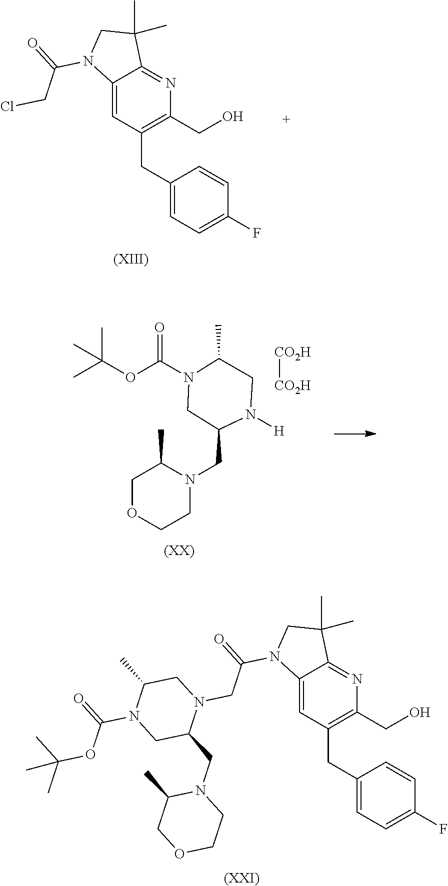

In an exemplary embodiment, the coupling reaction can occur with potassium iodide and potassium carbonate in a suitable solvent, such as acetonitrile.

The compound of formula (XXII) is produced by deprotecting the compound of formula (XXI) with a deprotection agent. This step is described in Example 18.

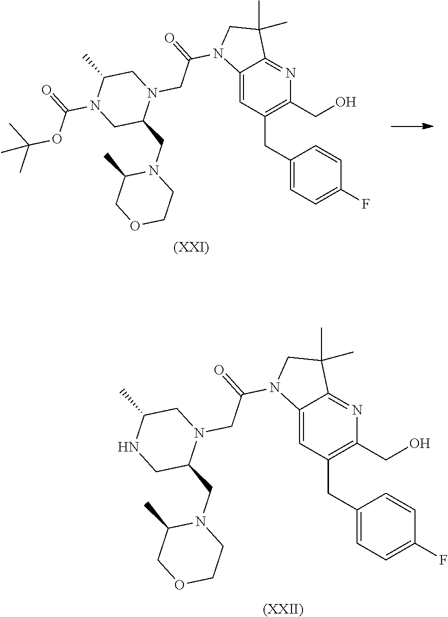

Suitable deprotection agents include, but are not limited to, iodine, hydrochloric acid, TFA, HBr, MsOH, TsOH, CSA or other acids. Deprotection can occur in a solvent, such as isopropyl alcohol, methanol, ethanol, t-butanol, THE or MeCN.

In a final and key step in the synthesis, the compound of formula (XXII) is reacted with anhydrous L-lactic acid to produce the end-product compound of formula (XXIII) that is more suitable and stable in pharmaceutical formulations.

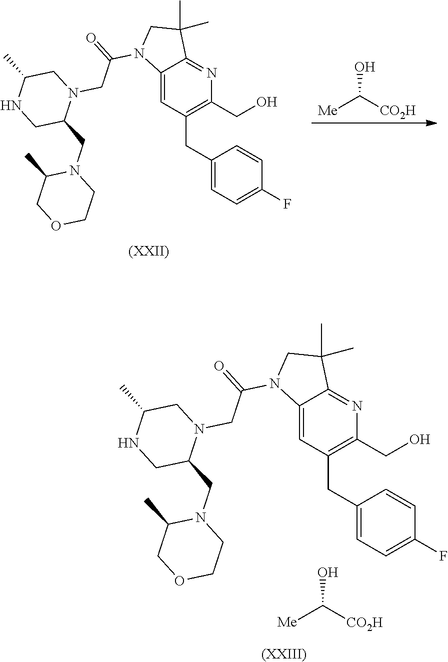

This final key step is described in Example 19 and produces an L-(+)-lactic acid salt of the compound of formula (XXIII), driving up the purity of the resulting compound and active pharmaceutical ingredient.

In an alternative embodiment, a key intermediate compound of formula (I) and methods of synthesizing a compound of formula (I) are provided:

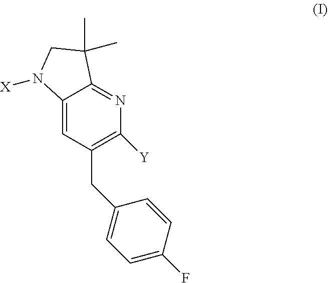

wherein X is H or a protecting group;

### Y is Br, Cl, I or COR; and

R is H, OH, O-alkyl or O-aryl. The protecting group can be a Boc protecting group.

In one embodiment provided herein is a compound of formula (I), or a salt, solvate or hydrate thereof,

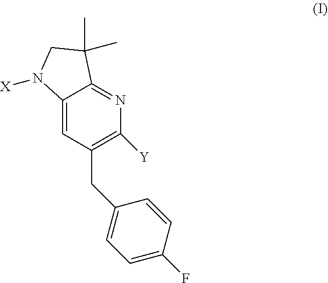

wherein X is H or a protecting group;

### Y is COR; and

### R is OH, O-alkyl or O-aryl.

FIG. 1 depicts a general scheme for the synthesis of the compound of formula (XXIII).

Embodiment 1 is a method of making a compound of formula (VII):

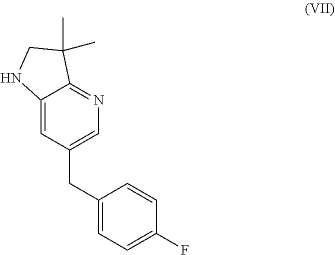 [SMILES: CC1(C)CNc2cc(Cc3ccc(F)cc3)cnc21]

comprising the steps of:
converting a compound of formula (III):

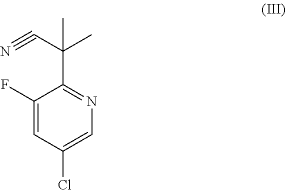

to the compound of formula (VII).

Embodiment 2 is a method of making the compound of formula (VII) described in embodiment 1, comprising borylating the compound of formula (III) with a borylating agent to produce a compound of formula (IIIa):

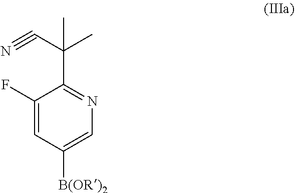

wherein each R′ is independently an H, alkyl, or aryl group or two alkyl or aryl groups forming a ring with B.

Embodiment 3 is a method of making the compound of formula (VII) described in embodiment 1, comprising borylating the compound of formula (III) in the presence of a palladium catalyst, Grignard reagent or alkyl lithium reagent.

Embodiment 4 is a method of making the compound of formula (VII) described in embodiment 1, comprising borylating the compound of formula (III) in the presence of a palladium catalyst and a ligand.

Embodiment 5 is a method of making the compound of formula (VII) described in embodiment 4, wherein the borylating agent is Y—B(OR′)₂, Y is OR′ or B(OR′)₂ and each R′ is independently an H, alkyl, or aryl group or two alkyl or aryl groups forming a ring with B.

Embodiment 6 is a method of making the compound of formula (VII) described in embodiment 3, wherein the palladium catalyst is a XPhos-Pd-G2 catalyst.

Embodiment 7 is a method of making the compound of formula (VII) described in embodiment 4, wherein the ligand is 2-dicyclohexylphosphino-2′,4′,6′-triisopropylbiphenyl.

Embodiment 8 is a method of making the compound of formula (VII) described in embodiment 1, further comprising benzylating the compound of formula (IIIa) with a benzylating agent to produce a compound of formula (IIIb):

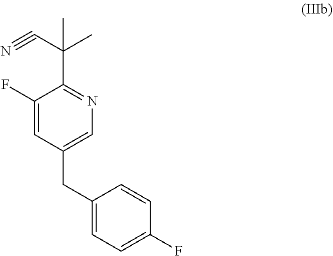 [SMILES: CC(C)(C#N)c1ncc(Cc2ccc(F)cc2)cc1F]

Embodiment 9 is a method of making the compound of formula (VII) described in embodiment 8, comprising benzylating the compound of formula (IIIa) in the presence of a base.

Embodiment 10 is a method of making the compound of formula (VII) described in embodiment 8, wherein the benzylating agent is 4-fluorobenzyl chloride or 4-fluoro benzyl bromide.

Embodiment 11 is a method of making the compound of formula (VII) described in embodiment 9, wherein the base is selected from the group consisting of potassium carbonate, sodium carbonate, sodium hydroxide, potassium hydroxide and potassium phosphate.

Embodiment 12 is a method of making the compound of formula (VII) described in embodiment 8, further comprising reducing the compound of formula (IIIb) with a reducing agent to produce a compound of formula (IIIc):

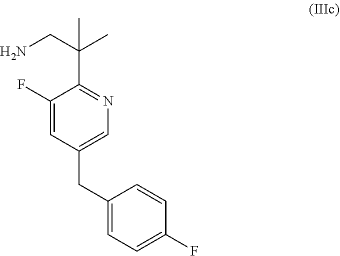 [SMILES: CC(C)(CN)c1ncc(Cc2ccc(F)cc2)cc1F]

Embodiment 13 is a method of making the compound of formula (VII) described in embodiment 12, comprising reducing the compound of formula (IIIb) in the presence of a nickel catalyst.

Embodiment 14 is a method of making the compound of formula (VII) described in embodiment 12, wherein the reducing agent is hydrogen gas, sodium borohydride or lithium aluminum hydride.

Embodiment 15 is a method of making the compound of formula (VII) described in embodiment 13, wherein the nickel catalyst is selected from the group consisting of nickel chloride, nickel (II) chloride hexahydrate and a Raney nickel catalyst.

Embodiment 16 is a method of making the compound of formula (VII) described in embodiment 12, further comprising deprotonating the compound of formula (IIIc) with a deprotonating agent.

Embodiment 17 is a method of making the compound of formula (VII) described in embodiment 16, further comprising cyclizing the compound of formula (IIIc) in the presence of a polar aprotic solvent.

Embodiment 18 is a method of making the compound of formula (VII) described in embodiment 16, wherein the deprotonating agent is sodium bicarbonate.

Embodiment 19 is a method of making the compound of formula (VII) described in embodiment 16, wherein the polar aprotic solvent is dimethyl sulfoxide.

Embodiment 20 provides a compound of formula (Ia):

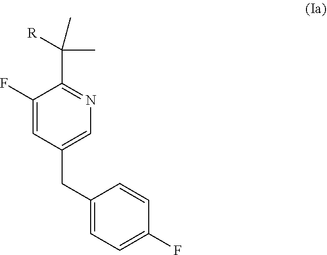

wherein R is CN or CH₂NH₂.

Embodiment 21 provides a method of making a compound of formula (VII):

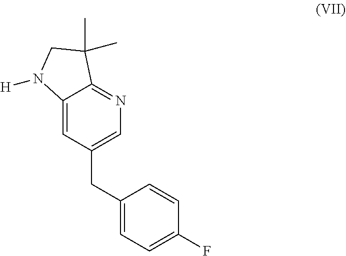 [SMILES: CC1(C)CNc2cc(Cc3ccc(F)cc3)cnc21]

comprising the steps of:
converting a compound of formula (V):

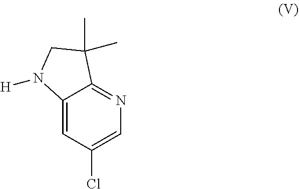

to the compound of formula (VII).

Embodiment 22 is a method of making the compound of formula (VII) described in embodiment 2, further comprising reacting the compound of formula (V) with a compound of formula (VI) in the presence of one or more palladium catalysts and a ligand.

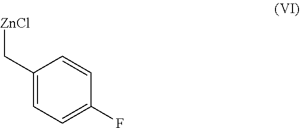

Embodiment 23 is a method of making the compound of formula (VII) described in embodiment 3, wherein the one or more palladium catalysts are selected from the group consisting of a XPhos-Pd-G2 catalyst, Pd(OAc)₂ and Pd₂(dba)₃.

Embodiment 24 is a method of making the compound of formula (VII) described in embodiment 4, wherein the ligand is selected from the group consisting of PPh₃, Xantphos, DPPE, DPPP, DPPB, DPPF, rac-BINAP, RuPhos, XPhos and tBuXphos.

Embodiment 25 provides a method of making a compound of formula (IX):

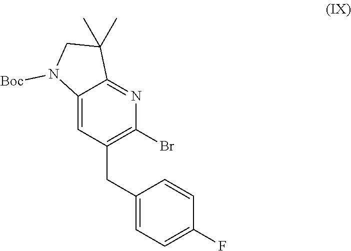 [SMILES: CC(C)(C)OC(=O)N1CC(C)(C)c2nc(Br)c(Cc3ccc(F)cc3)cc21]

comprising the steps of:
converting a compound of formula (V):

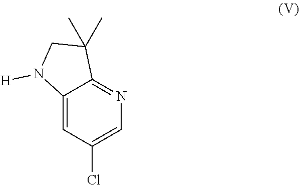

to the compound of formula (IX).

Embodiment 26 is a method of making the compound of formula (IX) described in embodiment 25, further comprising reacting the compound of formula (V) with a protecting group to produce a compound of formula (Va).

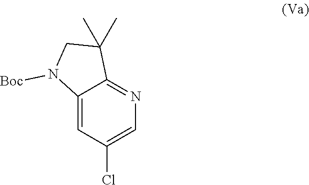 [SMILES: CC(C)(C)OC(=O)N1CC(C)(C)c2ncc(Cl)cc21]

Embodiment 27 is a method of making the compound of formula (IX) described in embodiment 26, wherein the protecting group is di-tert-butyl decarbonate.

Embodiment 28 is a method of making the compound of formula (IX) described in embodiment 26, further comprising borylating the compound of formula (Va) with a borylating agent to produce a compound of formula (Vb):

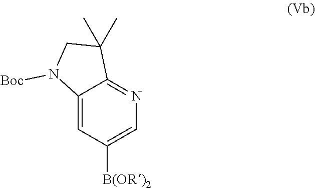

Embodiment 29 is a method of making the compound of formula (IX) described in embodiment 28, comprising borylating the compound of formula (Va) in the presence of palladium catalyst, Grignard reagent or alkyl lithium reagent.

Embodiment 30 is a method of making the compound of formula (IX) described in embodiment 28, comprising borylating the compound of formula (Va) in the presence of palladium catalyst and ligand.

Embodiment 31 is a method of making the compound of formula (IX) described in embodiment 29, wherein the borylating agent is Y—B(OR′)₂, Y is OR′ or B(OR′)₂ and each R′ is independently an H, alkyl, or aryl group or two alkyl or aryl groups forming a ring with B.

Embodiment 32 is a method of making the compound of formula (IX) described in embodiment 29, wherein the palladium catalyst is selected from the group consisting of Pd-Ln (Palladium-lanthanide compartment complex), Pd-170 (XPhos Pd(crotyl)Cl), XPhos-Pd-G2 catalyst, Pd(OAc)₂ and Pd₂(dba)₃.

Embodiment 33 is a method of making the compound of formula (IX) described in embodiment 30, wherein the ligand is 2-dicyclohexylphosphino-2′,4′,6′-triisopropylbiphenyl (XPhos).

Embodiment 34 is a method of making the compound of formula (IX) described in embodiment 28, further comprising benzylating the compound of formula (Vb) with a benzylating agent to produce a compound of formula (Vc):

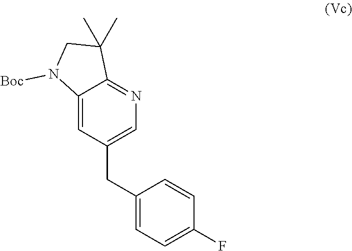 [SMILES: CC(C)(C)OC(=O)N1CC(C)(C)c2ncc(Cc3ccc(F)cc3)cc21]

Embodiment 35 is a method of making the compound of formula (IX) described in embodiment 34, comprising benzylating the compound of formula (Vb) in the presence of an inorganic base.

Embodiment 35 is a method of making the compound of formula (IX) described in embodiment 34, wherein the benzylating agent is 1-(chloromethyl)-4-fluorobenzene or 4-fluoro benzyl bromide.

Embodiment 37 is a method of making the compound of formula (IX) described in embodiment 35, wherein the inorganic base is selected from the group consisting of potassium carbonate, sodium carbonate, sodium hydroxide, potassium hydroxide and potassium phosphate.

Embodiment 38 is a method of making the compound of formula (IX) described in embodiment 34, further comprising brominating the compound of formula (Vc) with a brominating agent.

Embodiment 39 is a method of making the compound of formula (IX) described in embodiment 38, comprising brominating the compound of formula (Vc) in the presence of an organic solvent.

Embodiment 40 is a method of making the compound of formula (IX) described in embodiment 38, wherein the brominating agent is selected from the group consisting of 1-bromopyrrolidine-2,5-dione (BMS) and 1,3-Dibromo-5,5-dimethylhydantoin (DBDMH).

Embodiment 41 is a method of making the compound of formula (IX) described in embodiment 39, wherein the organic solvent is selected from the group consisting of dimethylformamide (DMF), dichloromethane, acetonitrile and ethyl acetate.

Embodiment 42 provides a compound of formula (I):

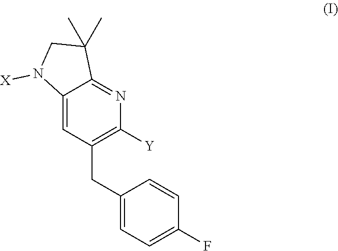

wherein X is H or a protecting group;

Y is Br, Cl, I or COR; and

R is H, OH, O-alkyl or O-aryl.

Embodiment 43 provides a compound of formula (I), wherein X is tert-butoxycarbonyl (Boc) and Y is Br.

Embodiment 44 provides a compound of formula (I), wherein the X is carboxybenzyl (Cbz).

Embodiment 45 provides a compound of formula (I), wherein X is Boc group and Y is CO₂Ph.

Embodiment 46 provides a compound of formula (I), wherein X is hydrogen and Y is CO₂Ph.

Embodiment 47 provides a method of making a compound of formula (I):

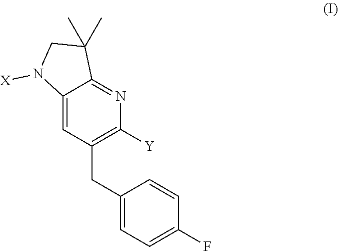

wherein X is H or a protecting group;

Y is Br, Cl, I or COR; and

R═H, OH, O-alkyl or O-aryl

comprising the steps of:
converting a compound of formula (VIII):

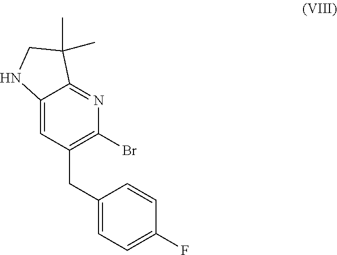 [SMILES: CC1(C)CNc2cc(Cc3ccc(F)cc3)c(Br)nc21]

to a compound of formula (I).

Embodiment 48 is a method of making the compound of formula (I) described in embodiment 47, further comprising reacting the compound of formula (VIII) with di-tert-butyl dicarbonate to produce a compound of formula (IX):

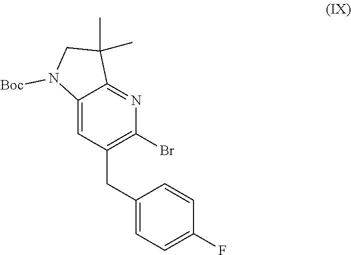 [SMILES: CC(C)(C)OC(=O)N1CC(C)(C)c2nc(Br)c(Cc3ccc(F)cc3)cc21]

Embodiment 49 is a method of making the compound of formula (I) described in embodiment 48, wherein reacting the compound of formula (VIII) with di-tert-butyl dicarbonate occurs in solution with one or more bases and one or more solvents.

Embodiment 50 is a method of making the compound of formula (I) described in embodiment 49, wherein the one or more bases are selected from the group consisting of sodium carbonate, N,N-dimethylamino pyridine, sodium hydroxide, triethylamine, sodium bicarbonate, potassium carbonate and diisopropylethylamine.

Embodiment 51 is a method of making the compound of formula (I) described in embodiment 49, wherein the one or more solvents are selected from the group consisting of toluene, dichloromethane, ethyl acetate and water.

Embodiment 52 is a method of making the compound of formula (I) described in embodiment 48, further comprising reacting the compound of formula (IX) with (i) phenyl formate or (ii) phenol and carbon monoxide in the presence of a palladium catalyst to produce a compound of formula (X):

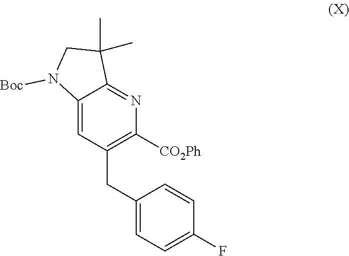

Embodiment 53 is a method of making the compound of formula (I) described in embodiment 52, wherein reacting the compound of formula (IX) occurs in solution with rac-1,1′-binaphthyl-2,2′-diphenyl phosphene. In some embodiments, the solution further comprises a base and solvent. In some embodiments, the solution further comprises triethylamine and acetonitrile.

Embodiment 54 is a method of making the compound of formula (I) described in embodiment 52, wherein carbon monoxide is in the gas phase.

Embodiment 55 is a method of making the compound of formula (I) described in embodiment 52, wherein the palladium catalyst is selected from the group consisting of XPhos-Pd-G2 catalyst, Pd(OAc)₂ and Pd₂(dba)₃.

Embodiment 56 is a method of making the compound of formula (I) described in embodiment 55, wherein reacting the compound of formula (IX) occurs in the presence of a ligand selected from the group consisting of PPh₃, Xantphos, DPPE, DPPP, DPPB, DPPF, rac-BINAP, RuPhos, tBuXphos and 2-dicyclohexylphosphino-2′,4′,6′-triisopropylbiphenyl (XPhos).

Embodiment 57 is a method of making the compound of formula (I) described in embodiment 52, further comprising reacting the compound of formula (X) with hydrochloric acid to produce a compound of formula (XI):

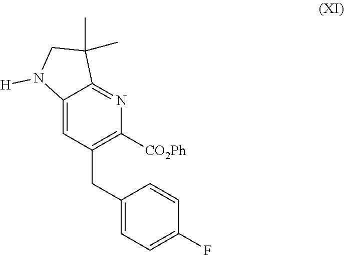

Embodiment 58 is a method of making the compound of formula (I) described in embodiment 52, wherein reacting the compound of formula (X) with hydrochloric acid occurs in solution with isopropanol.

Embodiment 59 is a method of making the compound of formula (I) as described in embodiment 57, further comprising reducing the compound of formula (XI) with a reducing agent to produce a compound of formula (XII):

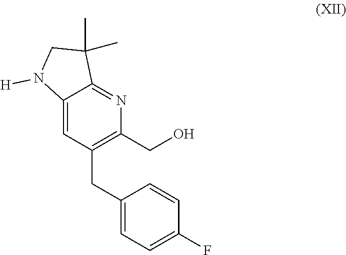 [SMILES: CC1(C)CNc2cc(Cc3ccc(F)cc3)c(CO)nc21]

Embodiment 60 is a method of making the compound of formula (I) as described in embodiment 59, wherein the reducing agent is selected from the group consisting of lithium borohydride, sodium borohydride, lithium aluminum hydride, borane, sodium triacetoxy borohydride, L-selectride, K-selectride, Red-Al and DIBAL.

Embodiment 61 provides a method of making a compound of formula (XIII):

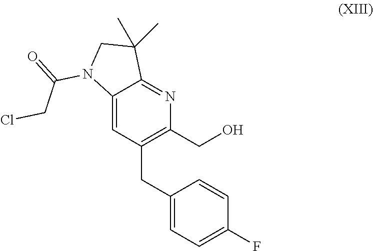 [SMILES: CC1(C)CN(C(=O)CCl)c2cc(Cc3ccc(F)cc3)c(CO)nc21]

comprising the steps of:
converting a compound of formula (XII):

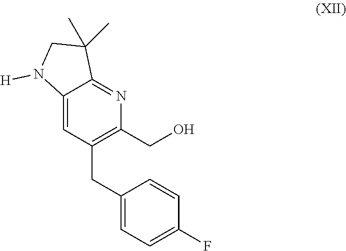 [SMILES: CC1(C)CNc2cc(Cc3ccc(F)cc3)c(CO)nc21]

to the compound of formula (XIII).

Embodiment 62 is a method of making the compound of formula (XIII) described in embodiment 61, further comprising reacting the compound of formula (XII) with 2-chloroacetyl chloride.

Embodiment 63 is a method of making the compound of formula (XIII) described in embodiment 62, wherein reacting the compound of formula (XII) with 2-chloroacetyl chloride occurs in the presence of acetonitrile.

Embodiment 64 provides a compound of formula (XVIa):

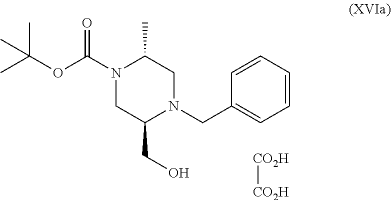

Embodiment 65 provides a method of making a compound of formula (XVIa):

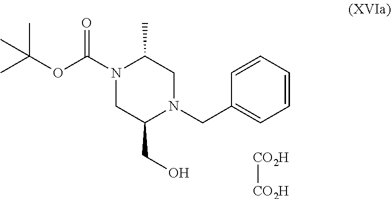

comprising the steps of:

converting a compound of formula (XIV):

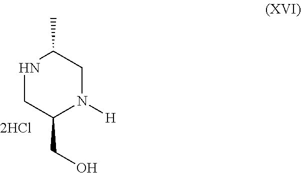

to a compound of formula (XVIa).

Embodiment 66 is a method of making the compound of formula (XVIa) described in embodiment 65, further comprising reacting the compound of formula (XIV) with di-tert-butyl dicarbonate to produce a compound of formula (XV):

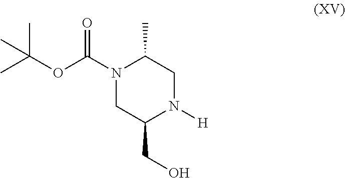 [SMILES: C[C@@H]1CN[C@@H](CO)CN1C(=O)OC(C)(C)C]

Embodiment 67 is a method of making the compound of formula (XVIa) described in embodiment 66, wherein reacting the compound of formula (XIV) with di-tert-butyl dicarbonate occurs in the presence of a base and a solvent.

Embodiment 68 is a method of making the compound of formula (XVIa) described in embodiment 67, wherein the base is selected from the group consisting of potassium hydroxide and sodium hydroxide.

Embodiment 69 is a method of making the compound of formula (XVIa) described in embodiment 67, wherein the solvent is selected from the group consisting of dichloromethane, ethyl acetate and ethanol.

Embodiment 70 is a method of making the compound of formula (XVIa) described in embodiment 66, further comprising benzylating the compound of formula (XV) with a benzylating agent to produce a compound of formula (XVI):

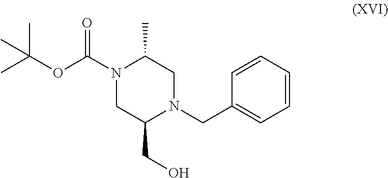 [SMILES: C[C@@H]1CN(Cc2ccccc2)[C@@H](CO)CN1C(=O)OC(C)(C)C]

Embodiment 71 is a method of making the compound of formula (XVIa) described in embodiment 70, wherein the benzylating agent is benzaldehyde.

Embodiment 72 is a method of making the compound of formula (XVIa) described in embodiment 70, wherein benzylating the compound of formula (XV) occurs in the presence of a reducing agent and a solvent.

Embodiment 73 is a method of making the compound of formula (XVIa) described in embodiment 72, wherein the reducing agent is selected from the group consisting of sodium triacetoxyborohydride, sodium borohydride (NaBH₄), borane and diisobutylaluminium hydride (DIBAL-H).

Embodiment 74 is a method of making the compound of formula (XVIa) described in embodiment 72, wherein the solvent is selected from the group consisting of dichloromethane, ethyl acetate and ethanol.

Embodiment 75 is a method of making the compound of formula (XVIa) described in embodiment 70, further comprising reacting the compound of formula (XVI) with oxalic acid in the presence of a solvent.

Embodiment 76 is a method of making the compound of formula (XVIa) described in embodiment 75, wherein the solvent is methyl tert-butyl ether.

Embodiment 77 provides a compound of formula (XX):

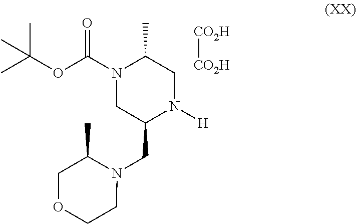

Embodiment 78 is a method of making the compound of formula (XX):

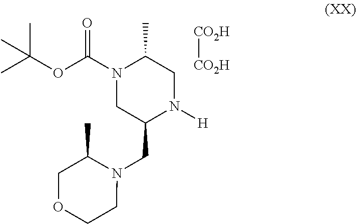

comprising the steps of:
converting a compound of formula (XVI) or a salt thereof:

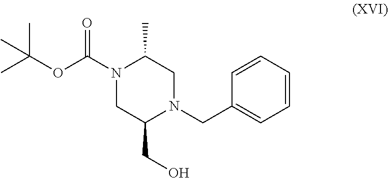 [SMILES: C[C@@H]1CN(Cc2ccccc2)[C@@H](CO)CN1C(=O)OC(C)(C)C]

to a compound of formula (XX).

Embodiment 79 is a method of making the compound of formula (XX) described in embodiment 78, further comprising chlorinating the compound of formula (XVI) or a salt thereof with a chlorinating agent to produce a compound of formula (XVII):

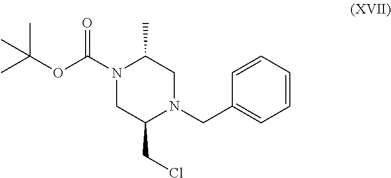 [SMILES: C[C@@H]1CN(Cc2ccccc2)[C@@H](CCl)CN1C(=O)OC(C)(C)C]

Embodiment 80 is a method of making the compound of formula (XX) described in embodiment 79, wherein the chlorinating agent is selected from the group consisting of thionyl chloride, sufuryl chloride and phosphoryl chloride (POCl₃).

Embodiment 81 is a method of making the compound of formula (XX) described in embodiment 79, wherein the chlorinating agent is methanesulfonyl chloride

Embodiment 82 is a method of making the compound of formula (XX) described in embodiment 79, wherein chlorinating the compound of formula (XVI) or a salt thereof occurs in the presence of a base and a solvent.

Embodiment 83 is a method of making the compound of formula (XX) described in embodiment 82, wherein the base is triethylamine.

Embodiment 84 is a method of making the compound of formula (XX) described in embodiment 82, wherein the solvent is selected from the group consisting of dichloromethane, ethyl acetate and ethanol.

Embodiment 85 is a method of making the compound of formula (XX) described in embodiment 79, further comprising reacting the compound of formula (XVII) with a nucleophile to produce a compound of formula (XIX):

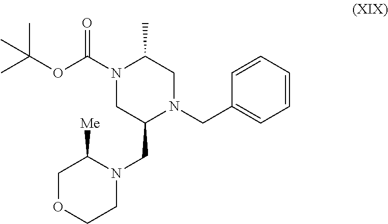 [SMILES: C[C@@H]1COCCN1C[C@H]1CN(C(=O)OC(C)(C)C)[C@H](C)CN1Cc1ccccc1]

Embodiment 86 is a method of making the compound of formula (XX) described in embodiment 85, wherein the nucleophile is (R)-3-methyl morpholine hydrochloride.

Embodiment 87 is a method of making the compound of formula (XX) described in embodiment 85, wherein reacting the compound of formula (XVII) with a nucleophile occurs in the presence of a base, a solvent and an additive.

Embodiment 88 is a method of making the compound of formula (XX) described in embodiment 87, wherein the base is selected from the group consisting of potassium carbonate, sodium carbonate and potassium phosphate.

Embodiment 89 is a method of making the compound of formula (XX) described in embodiment 87, wherein the solvent is acetonitrile and the additive is potassium iodide.

Embodiment 90 is a method of making the compound of formula (XX) described in embodiment 85, further comprising debenzylation of the compound of formula (XIX) to produce the compound of formula (XXa).

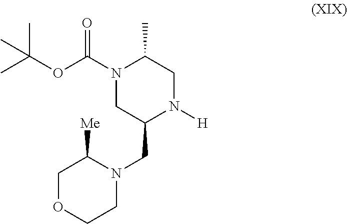 [SMILES: C[C@@H]1COCCN1C[C@H]1CN(C(=O)OC(C)(C)C)[C@H](C)CN1]

Embodiment 91 is a method of making the compound of formula (XX) described in embodiment 90, wherein debenzylation of the compound of formula (XIX) comprises reacting the compound of formula (XIX) with hydrogen and one or more palladium catalysts.

Embodiment 92 is a method of making the compound of formula (XX) as described in embodiment 91, wherein the palladium catalyst is selected from the group consisting of palladium on activated carbon and palladium hydroxide.

Embodiment 93 is a method of making the compound of formula (XX) described in embodiment 91, wherein debenzylation of the compound of formula (XIX) occurs in the presence of a solvent.

Embodiment 94 is a method of making the compound of formula (XX) described in embodiment 93, wherein the solvent is selected from the group consisting of benzene, methanol, toluene and heptane.

Embodiment 95 is a method of making the compound of formula (XX) described in embodiment 93, wherein the solvent is anhydrous ethanol.

Embodiment 96 is a method of making the compound of formula (XX) described in embodiment 91, wherein the hydrogen is in the gas phase.

Embodiment 97 is a method of making the compound of formula (XX) described in embodiment 90, further comprising reacting the compound of formula (XXa) with oxalic acid.

Embodiment 98 is a method of making a compound of formula (XXIII):

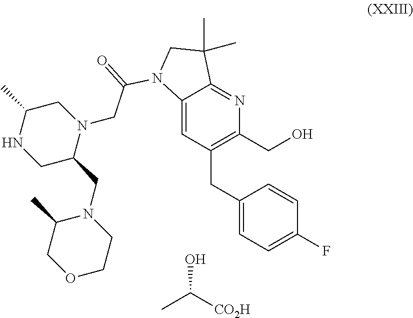

comprising the steps of:
converting a compound of formula (XX):

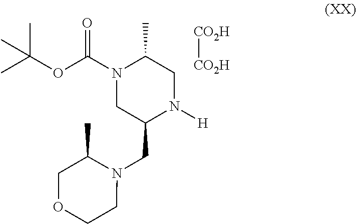

to the compound of formula (XXIII).

Embodiment 99 is a method of making the compound of formula (XXIII) described in embodiment 98, further comprising reacting the compound of formula (XX) with a compound of formula (XIII):

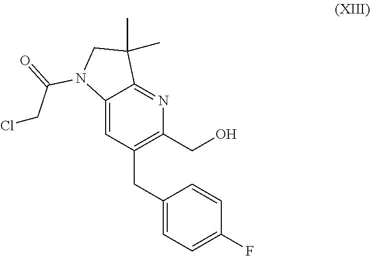 [SMILES: CC1(C)CN(C(=O)CCl)c2cc(Cc3ccc(F)cc3)c(CO)nc21]

to produce a compound of formula (XXI):

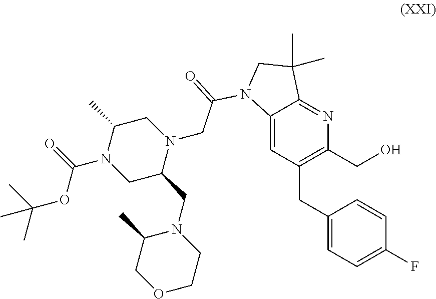 [SMILES: C[C@@H]1COCCN1C[C@H]1CN(C(=O)OC(C)(C)C)[C@H](C)CN1CC(=O)N1CC(C)(C)c2nc(CO)c(Cc3ccc(F)cc3)cc21]

Embodiment 100 is a method of making the compound of formula (XXIII) described in embodiment 99, wherein reacting the compound of formula (XX) with a compound of formula (XIII) occurs in the presence of potassium iodide, potassium carbonate and acetonitrile.

Embodiment 101 is a method of making the compound of formula (XXIII) described in embodiment 99, further comprising reacting the compound of formula (XXI) with a deprotection agent to produce a compound of formula (XXII)

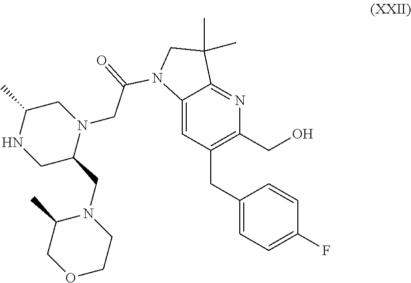 [SMILES: C[C@@H]1CN(CC(=O)N2CC(C)(C)c3nc(CO)c(Cc4ccc(F)cc4)cc32)[C@@H](CN2CCOC[C@H]2C)CN1]

Embodiment 102 is a method of making the compound of formula (XXIII) described in embodiment 101, wherein the deprotection agent is selected from the group consisting of iodine, TFA, HBr, MsOH, TsOH and CSA.

Embodiment 103 is a method of making the compound of formula (XXIII) described in embodiment 101, wherein the deprotection agent is HCl.

Embodiment 104 is a method of making the compound of formula (XXIII) described in embodiment 101, wherein reacting the compound of formula (XXI) with a deprotection agent occurs in the presence of a solvent.

Embodiment 105 is a method of making the compound of formula (XXIII) described in embodiment 104, wherein the solvent is selected from the group consisting of isopropyl alcohol, methanol, ethanol, t-butanol, THE and MeCN.

Embodiment 106 is a method of making the compound of formula (XXIII) described in embodiment 101, further comprising reacting the compound of formula (XXII) with anhydrous L-(+)-lactic acid.

Embodiment 107 is a method of making the compound of formula (XXIII) described in embodiment 106, wherein the purity of the compound of formula (XXIII) is equal to or greater than 95% by weight.

Embodiment 108 is a method of making the compound of formula (XXIII) described in embodiment 106, wherein the palladium content of the compound of formula (XXIII) is less than 10 ppm.

Embodiment 109 is a method of making the compound of formula (XXIII) described in embodiment 106, wherein the aldehyde impurity of the compound of formula (XXIII) represented by the area percent of RRT 1.3 is less than or equal to 0.15 area percent after storage for 12 months at 5° C. and 60% relative humidity.

Embodiment 110 is a method of making the compound of formula (XXIII) described in embodiment 106, further comprising reacting the compound of formula (XXII) with anhydrous L-(+)-lactic acid in the presence of one or more crystallization solvents.

Embodiment 111 is a method of making the compound of formula (XXIII) described in embodiment 110, wherein the one or more crystallization solvents is methyl isobutyl ketone (MIBK) and n-heptane.

Embodiment 112 is a method of making the compound of formula (XXIII) described in embodiment 110, wherein the one or more crystallization solvents is isopropanol and n-heptane.

Embodiment 113 is a method of making the compound of formula (XXIII) described in embodiment 110, wherein the one or more crystallization solvents is methyl ethyl ketone (MEK) and n-heptane.

Embodiment 114 is a method of making the compound of formula (XXIII) described in embodiment 110, wherein the one or more crystallization solvents is tetrahydrofuran (THF) and n-heptane.

Embodiment 115 is a method of making the compound of formula (XXIII) described in embodiment 110, wherein the one or more crystallization solvents is acetonitrile and methyl tert-butyl ether (MTBE).

Embodiment 116 is a method of making the compound of formula (XXIII) described in embodiment 110, wherein the one or more crystallization solvents is methyl acetate and n-heptane.

Embodiment 117 is a method of making the compound of formula (XXIII) described in embodiment 110, wherein the one or more crystallization solvents is ethyl acetate and n-heptane.

Embodiment 118 is a method of making the compound of formula (XXIII) described in embodiment 110, further comprising seeding the reaction with lactic acid salt crystal compounds of formula (XXIII)

Embodiment 119 is a method of making the compound of formula (XXIII) described in embodiment 118, wherein seeding the reaction occurs at a temperature of less than or equal to 60° C.

Embodiment 120 is a method of making the compound of formula (XXIII) described in embodiment 118, further comprising cooling the reaction at a rate of about 0.01° C./min to 1° C./min.

Embodiment 121 is a method of making the compound of formula (XXIII) described in embodiment 118, further comprising cooling the reaction at a rate of about 0.03° C./min to 0.3° C./min.

Problems of the known processes for the preparation of the compounds of formulas (XXIII) and (XXIIIa) are addressed by the present application and the embodiments and examples disclosed herein. Referring to FIG. 2, the inventors have unexpectedly discovered that the hydroxymethyl group of compounds of formula (XII) can be synthesized using a palladium catalyzed carbonylation to prepare a key ester intermediate compound of formula (X) in highly crystalline form followed by reduction. In addition, the embodiments and examples herein describe new intermediates, chemical entities and methods of synthesizing the new chemical entities and intermediates. For example, compounds of formulas (Ia), (I), (IX), (X), (XI), oxalate salt compound of formula (XVIa) and (XX) are new chemical entities that can be used in the preparation and synthesis of the compounds of formulas (XXIII) and (XXIIIa).

The synthesis of FIG. 2 and other exemplary embodiments also provide new chemical entities in the form of crystalline salts of formulas (XVI) and (XX) and new methods of synthesizing crystalline salt compounds of these formulas. Crystalline salts of the compounds of formulas (XVI) and (XX) have higher purity than non-crystalline salt forms or free base forms and improve the purity of the synthesis of end-product compounds of formulas (XXIII) and (XXIIIa). In an embodiment, the compound of formula (XX) is in oxalate salt form and used in oxalate salt form in the synthesis of the compound of formulas (XXIII).

Certain advantages are provided by using novel intermediate compounds of formulas (Ia), (Va), (VII), (IX), (X) and (XI) in the synthesis of the end-product compound of formula (XXIII) and other compounds of formula (XXIIIa). Intermediates of formulas (Ia), (Va), (VII), (IX), (X) and (XI) offer greater efficiency, predictability, isolation, purity and stability of the synthesis of the end-product compound of formula (XXIII). Further advantages are provided by using novel intermediate compounds of formulas (IX), (X), (XI), (XVI), (XVIa) and (XX) in the synthesis of the end-product compound of formula (XXIII) and other compounds of formula (XXIIIa). Intermediates of formulas (IX), (X), (XI), (XVI), (XVIa) and (XX) offer greater efficiency, predictability, isolation and purity of the synthesis of the end-product compound of formula (XXIII).

Problems of the known processes for the preparation of the compound of formula (XXIII) and other compounds of formula (XXIIIa) are addressed by the embodiments and examples of the present application. This application discloses an unexpected improvement in the synthesis of the compounds of formulas (IX), (X), (XI), (XVI), (XVIa) and (XX) and synthesis of the end-product compound of formula (XXIII). The hydroxymethyl group in the compound of formula (XII) is installed using a palladium catalyzed carbonylation of the compound of formula (IX) to prepare a key ester intermediate compound of formula (X) in highly crystalline form followed by reduction to create a high-purity, stable end-product compound of formula (XXIII) that is less tacky and more suitable in pharmaceutical formulations.

The examples below describe exemplary reaction conditions, parameters and reagents to carry out exemplary steps in the synthesis of the compounds of formulas (IX), (X), (XI), (XVI), (XVIa), (XX), end-product compound of formula (XXIII) and other compounds of formula (XXIIIa). The following examples are illustrative of some of the embodiments described herein. Those of ordinary skill in the art will understand that various alterations to the examples may exist without departing from the scope or intent of the present application or exemplary embodiments disclosed, including variations with respect to the synthesis methods, processes, reactants, reagents, parameters and conditions described herein. In the Examples below the acronym “NMT” stands for “not more than”

### Abbreviations

BOC or Boc Tert-butyloxy carbonyl
tBuXphos 2-Di-tert-butylphosphino-2′,4′,6′-triisopropylbiphenyl
DCM dichloromethane
DMF dimethyl formamide
DMSO dimethyl sulfoxide
DPPB 1,4-Bis(diphenylphosphino)butane
DPPE 1,2-Bis(diphenylphosphino)ethane
DPPF 1,1′-Ferrocenediyl-bis(diphenylphosphine)
DPPP 1,3-Bis(diphenylphosphino)propane
EtOAc ethyl acetate
EtOH ethanol
HCl hydrochloric acid
HBr hydrobromic acid
IPA isopropyl alcohol
KF Karl Fisher test of hygroscopicity
Me methyl
MeCN or ACN acetonitrile
MEK methyl ethyl ketone
MeOH methanol
MsOH methanesulfonic acid
MIBK methyl isobutyl ketone
MTBE or TBME methyl tert butyl ether
NBS N-bromosuccinimide
NMP N-methylpyrrolidine
NMT no more than
Ph Phenyl
Pd(OAc)₂ palladium acetate
Pd₂(dba)₃ Tris(dibenzylideneacetone)dipalladium(O)
PPh₃ triphenyl phosphine
ppm parts per million
rac-BINAP (±)-2,2′-Bis(diphenylphosphino)-1,1′-binaphthalene
RRT relative retention time
RH relative humidity
RuPhos 2-Dicyclohexylphosphino-2′,6′-diisopropoxybiphenyl
TFA trifluoroacetic acid
THF tetrahydrofuran
2-Me-THF 2-methyl tetrahydrofuran
TLC thin-layer chromatography
TsOH toluenesulfonic acid
Xantphos 4,5-Bis(diphenylphosphino)-9,9-dimethylxanthene
XPhos 2-dicyclohexylphosphino-2′,4′,6′-triisopropylbiphenyl
XPhos-Pd-G2 chloro(2-dicyclohexylphosphino-2′,4′,6′-triisopropyl-1,1′-biphenyl)[2-(2′-amino-1,1′-biphenyl)]palladium(II) EXAMPLES

### Example 1: Preparation and Synthesis of the Compound of Formula (IIIb)

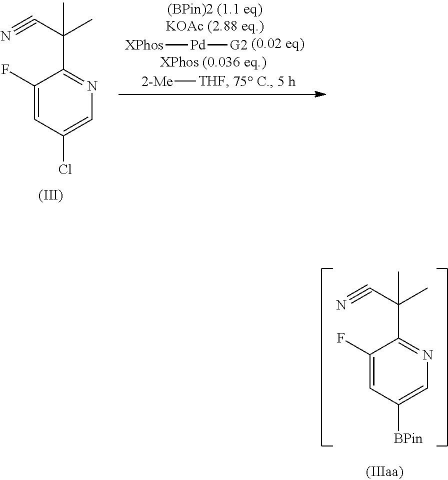

Potassium acetate (11.38 g, 115.97 mmol, 2.88 eq.), bis(pinacolato)diboron (11.35 g, 44.70 mmol, 1.11 eq.), XPhos-Pd-G2 (0.633 g, 0.80 mmol, 0.02 eq.), XPhos (0.69 g, 1.45 mmol, 0.036 eq.) and the compound of formula (III) (8.0 g, 40.27 mmol. 1.0 eq.) were added to a dry 3-neck flask under nitrogen. 2-Me-THF (120 mL) was added, the reaction mixture was heated to a temperature of 75° C. for 5 hours (until the disappearance of the compound of formula (III)), and the reaction mixture was cooled to a temperature of 60° C.

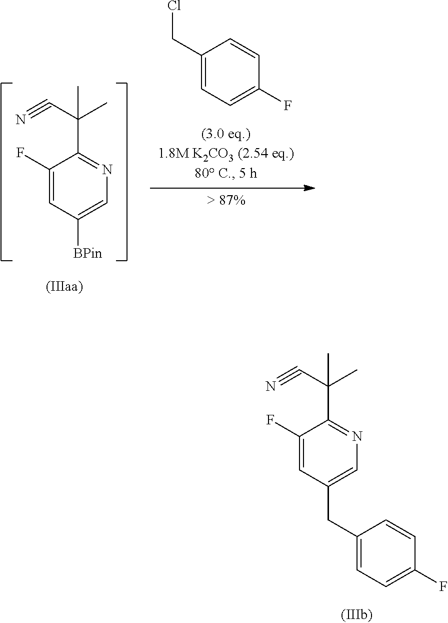

4-Fluorobenzyl chloride (17.46 g, 14.47 mL, 120.8 mmol, 3.0 eq.) was added to the reaction mixture followed by a dropwise (over 1 hour) addition of aqueous potassium carbonate solution (57 mL, 1.8 M, 102.28 mmol, 2.54 eq.). The reaction mixture was then stirred at a temperature of 60° C. for 5 additional hours until the completion of boronate as determined by thin-layer chromatography (TLC). The reaction mixture was then cooled to room temperature and transferred to a separatory funnel. The organic layer was separated, and the aqueous layer was extracted with EtOAc (25 mL×3). The combined organic layer was washed with brine (25 mL), dried over anhydrous sodium sulfate, filtered and concentrated. The crude product was then purified by column chromatography (Hexanes:EtOAc=20:1 to 15:1 to 10:1) to obtain 9.56 g (35.15 mmol, 87.3% by weight) to produce a desired product as light-yellow oil. ¹H-NMR (CDCl₃) analysis of the product produced the following results: δ 8.26 (1H, s), 7.20 (1H, dd), 7.15 (2H, m), 7.03 (2H, t), 3.99 (2H, s), 1.80 (6H, s).

### Example 2: Preparation and Synthesis of the Compound of Formula (IIIc)

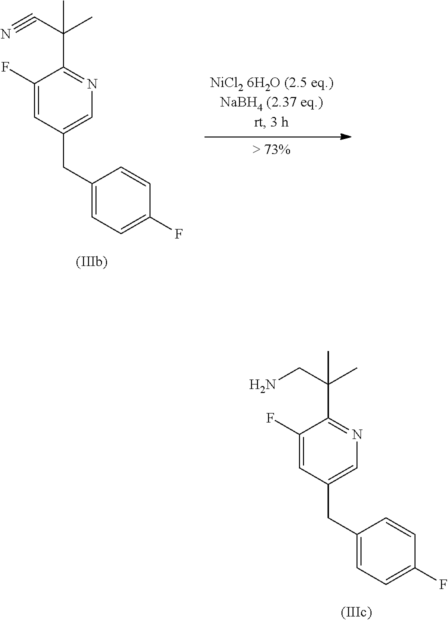 [SMILES: CC(C)(C#N)c1ncc(Cc2ccc(F)cc2)cc1F ; CC(C)(CN)c1ncc(Cc2ccc(F)cc2)cc1F]

NiCl₂·6H₂O (10.90 g, 45.9 mmol, 2.5 eq.) was added to a solution of the compound of formula (IIIb) (5.0 g, 18.36 mmol, 1.0 eq.) in MeOH (60 mL). The reaction flask was transferred to an ice bath and NaBH₄ (1.64 g, 43.5 mmol, 2.37 eq.) was added portion wise over 15 minutes. The reaction mixture was stirred at a temperature of 0° C. for 15 minutes and then at room temperature for additional 3 hours (until the disappearance of starting material by TLC). The reaction was then cooled in an ice bath and quenched by a dropwise addition of 30% ammonia solution (50 mL). The reaction mixture was then filtered through Celite®, the filter cake was washed with MeOH (10 mL×3) and the filtrate was concentrated. Then, 15 mL of 30% ammonia solution was added to the reaction mixture and extracted with dichloromethane (50 mL) and (4×20 mL). The combined organic extract was washed with brine (25 mL), dried over anhydrous sodium sulfate, filtered and concentrated. The crude reaction product was then purified by column chromatography (DCM:MeOH=10:1, 1% Et₃N) to obtain 3.68 g (73% by weight) of the desired product as light-yellow viscous oil. ¹H-NMR (CDCl₃) analysis of the product produced the following results: δ 8.22 (1H, s), 7.15 (2H, m), 7.07 (1H, dd), 7.02 (2H, t), 3.94 (2H, s), 3.01 (2H, s), 1.37 (6H, s).

### Example 3: Preparation and Synthesis of the Compound of Formula (VII)

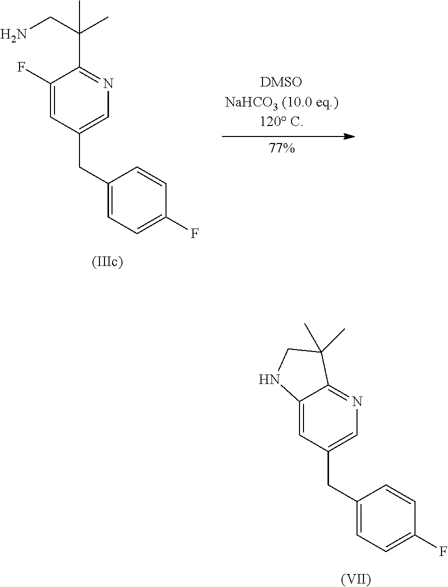 [SMILES: CC(C)(CN)c1ncc(Cc2ccc(F)cc2)cc1F ; CC1(C)CNc2cc(Cc3ccc(F)cc3)cnc21 ; "(VII)"]

Solid NaHCO₃ (11.2 g, 113.0 mmol, 10.0 eq.) was added to a solution of the compound of formula (IIIc) (3.68 g, 13.3 mmol, 1.0 eq.) in DMSO (55 ml). The slurry was then heated to a temperature of 120° C. for 48 hours. TLC showed small amounts of unreacted starting compound of formula (IIIc). NaHCO₃ (2.24 g, 22.5 mmol, 2.0 eq.) was added, and the reaction mixture was stirred at 120° C. for an additional 4 hours. Trace amounts of unreacted starting material was detected by TLC. The crude reaction mixture was then slowly poured into 50 mL of ice/water and the resulting yellow precipitate was then filtered via a Buchner funnel. The filter cake was washed with ice water (20 mL), and the solid was dried at 45° C. for 16 hours to obtain 2.61 g (77%) of crude product as a yellowish solid. ¹H-NMR (CDCl₃) analysis of the product produced the following results: δ 7.78 (1H, s), 7.14 (2H, m), 6.97 (2H, t), 6.56 (1H, s), 3.82 (2H, s), 3.66 (2H, s), 3.37 (2H, s), 1.34 (6H, s).

### Example 3A: Preparation and Synthesis of the Compound of Formula (VII)

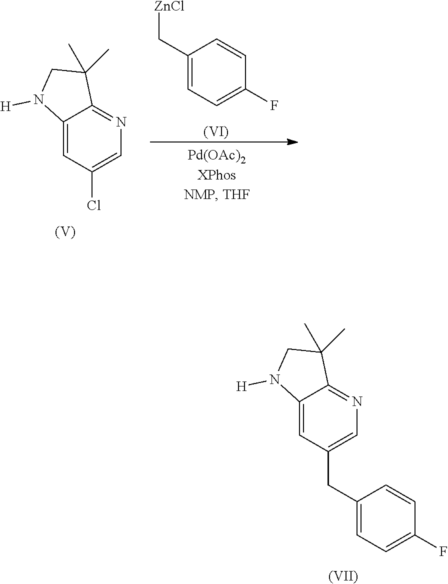

Lithium bromide (71.32 g, 3.0 eq), palladium acetate (0.614 g, 0.01 eq) and XPhos (3.39 g, 0.026 eq) were added to a solution of the compound of formula (V) (50 g, 1 eq) in N-methyl-pyrrolidone (100 mL) and tetrahydrofuran (150 mL). The reaction mixture was heated to a temperature of 30 to 36° C., and 4-fluorobenzyl zinc chloride (821 mL, 1.5 eq, 0.5 M in THF) was added. The reaction mixture was heated to a temperature of 30-36° C. for 12 hours. Upon reaction completion, the reaction mixture was cooled to a temperature of 15 to 25° C. and quenched with a 13% aqueous solution of ammonium chloride (220 mL). The reaction mixture was filtered, and the aqueous phase was extracted with toluene (250 mL). The combined organic layer was concentrated to about 1.05 L volume and washed twice with a 13% aqueous solution of ammonium chloride (220 mL) at a temperature of 45 to 55° C. The organic layer was concentrated to about 200 mL volume and cooled to a temperature of 15 to 25° C. Heptane (500 mL) was added and stirred at a temperature of 15 to 25° C. for 30 min, filtered and washed with heptane (100 mL). The solid was dried under vacuum to a temperature of 40° C. for about 8 to 10 hours to obtain the compound of the formula (VII) (68.5 g, 81.2% yield, 98.5% area HPLC purity).

### Example 3B: Preparation and Synthesis of the Compound of Formula (VIII)

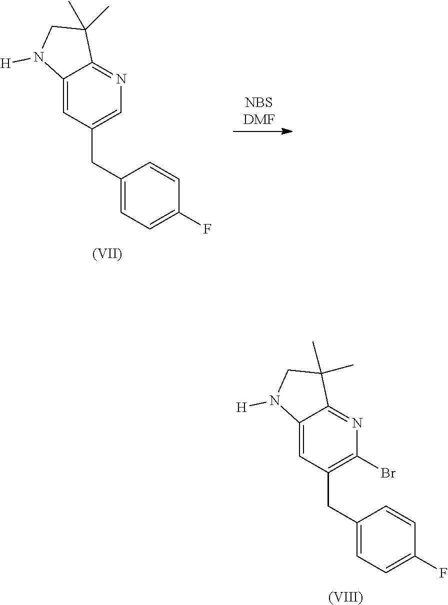 [SMILES: CC1(C)CNc2cc(Cc3ccc(F)cc3)cnc21 ; "(VII)" ; CC1(C)CNc2cc(Cc3ccc(F)cc3)c(Br)nc21 ; "(VIII)"]

A solution of N-Bromosuccinimide (21.4 Kg, 1.01 eq) in dimethylformamide (178 Kg) was added to a solution of the compound of formula (VII) (32.4 Kg, 1 eq) in dimethyl formamide (207 L) at a temperature of −18 to −12° C. The reaction mixture was stirred at this temperature for 1 hour, and upon reaction completion, water (455 Kg) was added. The resulting solids were filtered and washed with a mixture of dimethylformamide (95 Kg) with water (95 Kg) and again with water (196 Kg). The solids were dried under vacuum at a temperature of 50° C. for about 12 hours to obtain the compound of formula (VIII) (39.2 Kg, 84% yield, 98.6% area HPLC purity).

### Example 4: Preparation and Synthesis of the Compound of Formula (Va)

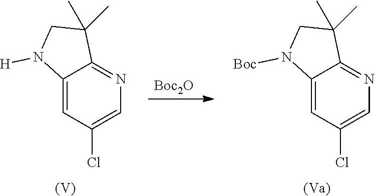

BOC-anhydride (21.28 ml, 92 mmol) was added portion wise to a rapidly stirring mixture of the compound of formula (V) (6-chloro-3,3-dimethyl-2,3-dihydro-1H-pyrrolo[3,2-b]pyridine) (10 g, 54.7 mmol) in THE (100 ml) and sodium carbonate 8% w/w in water (150 ml, 143 mmol). The reaction mixture was left stirring overnight. Triethylamine (TEA) (20 ml, 143 mmol) and BOC-anhydride (10.64 ml, 45.8 mmol) were added and stirred for another 24 hours. Approximately 40% conversion occurred. The mixture was partitioned with EtOAc (200 ml) and water (200 ml). The organics were separated, dried (MgSO₄), filtered, and the solvent was removed to provide a thick oil. Analysis of this oil after 16 h showed complete conversion to the desired compound. The compound was purified by flash chromatography on silica gel (220 g cartridge, 0-10% TBME/isohexane) to produce the compound of formula (Va) (tert-butyl 6-chloro-3,3-dimethyl-2,3-dihydro-1H-pyrrolo[3,2-b]pyridine-1-carboxylate) (13 g, 44.6 mmol, 81% yield) as a colorless oil. ¹H-NMR in CDCl₃ was consistent with product structure at 97% purity and approximately 3% w/w isohexane. ¹H-NMR (500 MHz, Chloroform-d) produced the following results: δ 8.08 (d, J=2.0 Hz, 1H), 7.54 (d, J=48.7 Hz, 1H), 3.78 (s, 2H), 1.83-1.45 (m, 9H), 1.39 (s, 6H).

### Example 5: Preparation and Synthesis of the Compound of Formula (Vc)

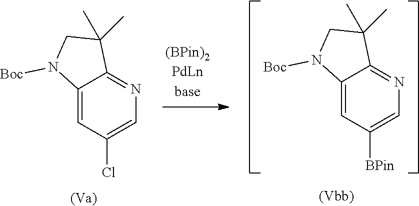

Bis(pinacolato)diboron (BPin)₂ (5 g, 19.69 mmol), Pd-170 (XPhos Pd(crotyl)Cl (250 mg, 0.371 mmol), XPhos (300 mg, 0.629 mmol) and potassium acetate (5 g, 50.9 mmol) were placed in a 3-neck flask, which was evacuated by backfilling with nitrogen three times. A solution of the compound of formula (Va) (tert-butyl 6-chloro-3,3-dimethyl-2,3-dihydro-1H-pyrrolo[3,2-b]pyridine-1-carboxylate) (5 g, 17.68 mmol) in 2-MeTHF (50 ml) was added, and the mixture was evacuated by backfilling with nitrogen three times before stirring under nitrogen at a temperature of 75° C. (internal temperature) for 30 min (or until conversion of starting material by UPLC).

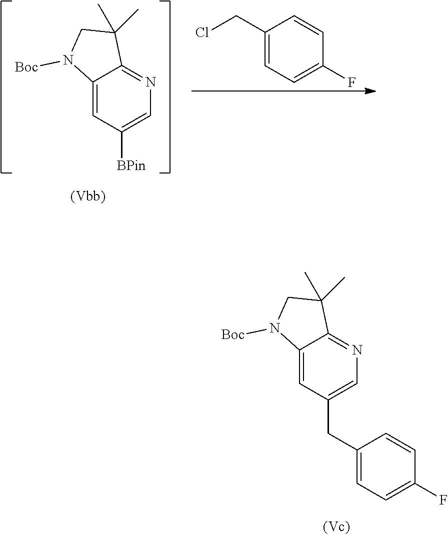

Formula (Vbb) is also drawn herein as

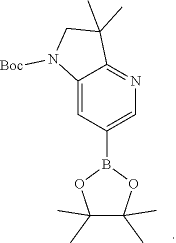 [SMILES: CC(C)(C)OC(=O)N1CC(C)(C)c2ncc(B3OC(C)(C)C(C)(C)O3)cc21]

Potassium carbonate 1.8M (25 ml, 45.0 mmol) was added followed by 1-(chloromethyl)-4-fluorobenzene (2.5 ml, 20.75 mmol), and stirring under nitrogen at 75° C. was continued for 4 hours. The reaction was cooled to ambient temperature and the organics were separated. The aqueous phase was extracted with EtOAc (50 ml). The organics were bulked and dried with MgSO₄ and filtered and preabsorbed onto silica (10 g) for purification by chromatography on silica gel (80 g cartridge, 0-20% EtOAc/isohexane) to produce the compound of formula (Vc) (tert-butyl 6-(4-fluorobenzyl)-3,3-dimethyl-2,3-dihydro-1H-pyrrolo[3,2-b]pyridine-1-carboxylate) (5.8 g, 15.46 mmol, 87% yield) as a tan gum. The product was analyzed by LCMS (Waters Acquity UPLC, X-Select, Waters X-Select UPLC C18, 1.7 μm, 2.1×30 mm, Acidic (0.1% Formic acid) 3 min method, 5-95% MeCN/water) and produced the following results: 2370-69-2A, m/z 357.2 (M+H)+ (ES+); at 1.77 min, 95% purity (diode array). ¹H-NMR in CDCl₃ 2370-69-2A was consistent with product structure at 95% purity of 4% w/w EtOAc, 1% w/w isohexane. ¹H-NMR (500 MHz, Chloroform-d) produced the following results: δ 8.01 (s, 1H), 7.91&7.21 (2×s, 1H, rotomers), 7.21-7.13 (m, 2H), 7.00 (s, 2H), 3.92 (s, 2H), 3.75 (s, 2H), 1.53 (d, J=14.2 Hz, 9H), 1.39 (s, 6H).

### Example 6: Preparation and Synthesis of the Compound of Formula (IX) (tert-butyl 5-bromo-6-(4-fluorobenzyl)-3,3-dimethyl-2,3-dihydro-1H-pyrrolo[3,2-b]pyridine-1-carboxylate)

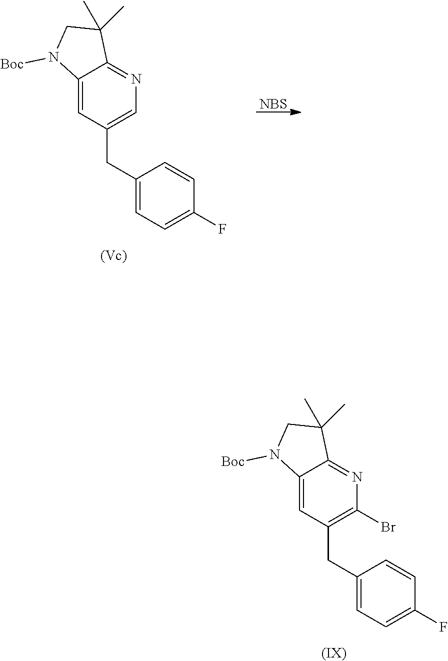

The compound of formula (Vc) (tert-butyl-6-(4-fluorobenzyl)-3,3-dimethyl-2,3-dihydro-1H-pyrrolo[3,2-b]-pyridine-1-carboxylate) (0.25 g, 0.70 mmol) was dissolved in DMF (12 mL) and cooled in an NaCl/ice bath to a temperature of 0° C. 1-bromopyrrolidine-2,5-dione (0.125 g, 0.70 mmol) was added dropwise as a solution in DMF (2 mL) over 5 mins. The reaction was left to warm to room temperature and stirred for 60 hours. The reaction was poured into brine (40 mL) and extracted with TBME (2×20 mL). The combined organic layers were concentrated directly onto silica. The crude product was purified by chromatography on silica gel (12 g cartridge, 0-20% EtOAc/isohexane) to produce the compound of formula (IX) (190 mg, 0.43 mmol, 61.6% yield) as a white solid. ¹H-NMR (500 MHz, DMSO-d₆) analysis produced the following results: δ 7.85 (s, 1H), 7.27 (s, 2H), 7.16 (t, J=8.7 Hz, 2H), 4.02 (s, 2H), 3.72 (s, 2H), 1.57-1.30 (m, 9H), 1.27 (s, 6H). m/z 435.1 & 437.1 (M+H)+(ES+), 99% purity (254 nm).

### Example 6A: Preparation and Synthesis of the Compound of Formula (IX) (tert-butyl-5-bromo-6-(4-fluorobenzyl)-3,3-dimethyl-2,3-dihydro-1H-pyrrolo[3,2-b]pyridine-1-carboxylate)

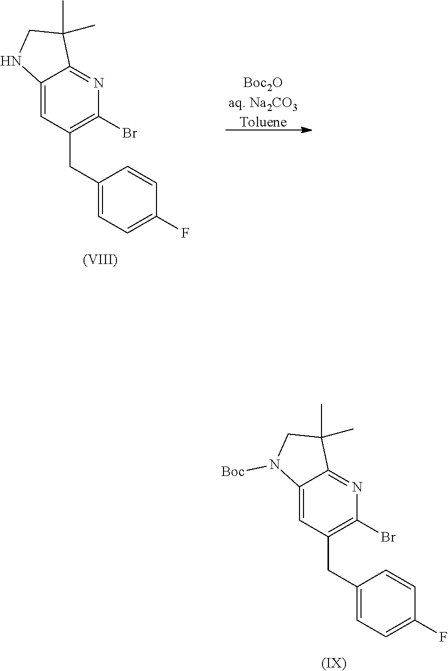

Di-tert-butyl dicarbonate (68.0 Kg, 2.57 eq) was added to a solution of the compound of formula (VIII) (41.6 Kg, 97.6% assay, 99.1% purity, 1 eq) in toluene (143.7 Kg) at a temperature of 15 to 25° C. The mixture was cooled to 0 to 10° C. and stirred at that temperature for 10-20 min. A solution of sodium carbonate (19.2 Kg) in purified water (164.2 Kg) was added to the mixture, and the mixture was stirred for 22 hours at a temperature of 15 to 25° C. until reaction completion. N,N-dimethylamino pyridine (0.4 Kg. 0.03 eq) was added to the mixture, and the resulting mixture was stirred for 12 hours at a temperature of 15 to 25° C. The organic phase was separated, and the aqueous phase was extracted with toluene (145 Kg). The combined organic phase was concentrated under vacuum (NMT 50° C.) to about 5 volumes. Toluene was swapped with methanol (4×320 Kg) until residual toluene was NMT 1% in about 5 volumes of methanolic solution. The mixture was cooled to a temperature of 15 to 25° C., and methanol (258 Kg) and water (132 Kg) were added. The mixture was stirred at this temperature for 7 hours, filtered, washed with methanol (64 Kg) and dried under vacuum at a temperature of 30 to 40° C. to obtain the compound of formula (IX) (47.98 Kg, 100% assay, 100% purity) as an off-white solid.

### Example 7: Preparation and Synthesis of Compound of Formula (X) (1-tert-butyl-5-phenyl-6-(4-fluorobenzyl)-3,3-dimethyl-2,3-dihydro-1H-pyrrolo[3,2-b]pyridine-1,5-dicarboxylate)

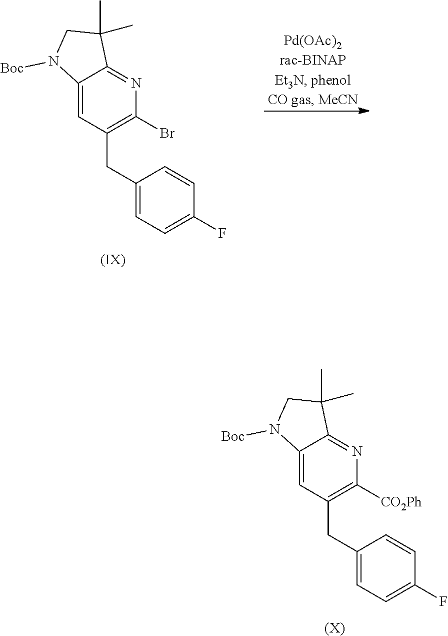

Phenol (33.20 Kg, 3.5 eq), palladium(II) acetate (0.7 Kg, 0.03 eq), rac-1,1′-binaphthyl-2,2′-diphenyl phosphene (1.9 Kg, 0.03 eq) and triethylamine (30.0 Kg, 3.0 eq) were added to a solution of compound of formula (IX) (43.0 Kg, 99.8% assay, 99.9% purity, 1 eq) in acetonitrile (356 Kg) in a pressure reactor. The pressure reactor was sealed and purged with nitrogen gas then exchanged with carbon monoxide gas to 0.03 to 0.05 MPa pressure. The reaction mixture was heated to a temperature of 55 to 65° C. and stirred at this temperature and pressure (0.03 to 0.05 MPa) for 33 hours until the compound of formula (IX) was NMT 1.0%. The reactor was purged with nitrogen gas and cooled to a temperature of 15 to 30° C., filtered and washed with acetonitrile (124 Kg). The filtrate was concentrated to about 5 volumes under vacuum at a temperature not exceeding 50° C. and swapped with ethanol (3×170 Kg) until residual acetonitrile was NMT 2.0%. The mixture was heated to a temperature of 45 to 50° C., and water (26 Kg) was added at this temperature. The mixture was stirred at this temperature for 4 hours and cooled to a temperature of 0 to 5° C., stirred at this temperature for 4 hours and filtered. The filter cake was washed with a mixture of ethanol (62 Kg) and water (7 Kg) and dried at a temperature of 40 to 45° C. under vacuum to obtain a crude material (638.5 Kg). The crude solid was dissolved in methyl tert-butyl ether (639 Kg) at a temperature of 15 to 25° C., filtered and rinsed with methyl tert-butyl ether (97 Kg). The filtrate was swapped with ethanol (2×170 Kg), distilling to about 5 volumes until the residual methyl tert-butyl ether was NMT 2%. The mixture was heated to a temperature of 70 to 80° C. and slowly cooled to 40 to 50° C. Water (25 Kg) was added at this temperature and cooled to 0 to 5° C., stirred at this temperature for 4 to 6 hours and filtered. The filter cake was washed with a mixture of ethanol (62 Kg) and water (7 Kg) and dried under vacuum at a temperature of 40 to 50° C. until residual ethanol was NMT 0.50% and KF was NMT 1%. The compound of formula (X) (38.8 Kg, 100% assay, 100% purity) was obtained as an off-white solid.

### Example 8: Preparation and Synthesis of Compound of Formula (XI) (phenyl-6-(4-fluorobenzyl)-3,3-dimethyl-2,3-dihydro-1H-pyrrolo[3,2-b]pyridine-5-carboxylate)

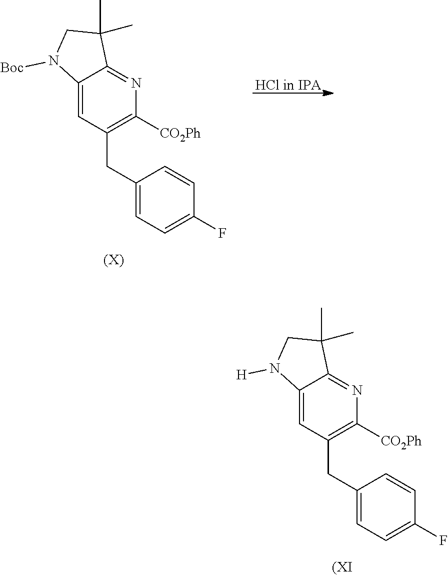

A solution of hydrochloric acid in isopropanol (75.6 Kg, 5.5 M) at a temperature of 50 to 55° C. was added to a solution of compound of formula (X) (31.8 Kg, 100% assay, 100% purity, 1 eq) in isopropanol (226 Kg). The reaction mixture was stirred at this temperature for 9 hours until reaction completion (compound of formula (X) NMT 0.5%). The reaction mixture was concentrated to about 4 volumes under vacuum at about 45° C. and then cooled to a temperature of 15 to 25° C. After adding 2-methyl tetrahydrofuran (191 Kg) and water (256 Kg), the pH was adjusted to 8 using aqueous sodium hydroxide. The aqueous layer was separated, and the organic layer was washed with brine (170 Kg). The combined aqueous layer was extracted with 2-methyl tetrahydrofuran (233 Kg), and the combined organic layer was concentrated to about 4 volumes under vacuum at about 45° C. Fresh 2-methyl tetrahydrofuran (3×240 Kg) was added and distilled to about 4 volumes until the water content was NMT 0.10%, thereby obtaining a 2-methyl tetrahydrofuran solution of the compound of formula (XI), which was telescoped into the next step.

### Example 9: Preparation and Synthesis of Compound of Formula (XII) (6-[(4-Fluorophenyl)methyl]-3,3-dimethyl-2,3-dihydro-1H-pyrrolo[3.2-b]pyridine-5-yl}methanol)

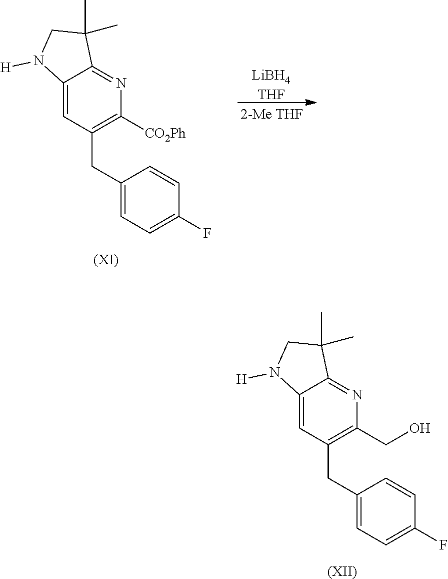

A solution of lithium borohydride (32.2 Kg, 1.05 eq) in THE at a temperature of −10 to 0° C. was added to a solution of the compound of formula (XI) (obtained from Example 5) in 2-methyl tetrahydrofuran at a temperature of −10 to 0° C. The reaction mixture was stirred at this temperature for 9 hours until reaction completion (unreacted compound of formula (XI) was NMT 1.0%). The reaction mixture was then added to a solution of potassium dihydrogen phosphate (38 Kg) in water (340 Kg). The organic phase was washed 3 times with aqueous sodium hydroxide until a pH of 12.5 to 13.0 was obtained. The organic phase was washed with aqueous potassium dihydrogen phosphate at a pH of 6.4 to 7.0. The organic phase was separated and swapped with toluene (2×220 Kg), distilling to about 3 volumes until residual 2-methyl tetrahydrofuran was NMT 2%. The mixture was then heated to a temperature of 70 to 75° C. and cooled gradually to a temperature of 0 to 5° C. over 4 to 5 hours. N-heptane (55 Kg) was added to the cold mixture and stirred at this temperature for 5 hours and filtered. The filter cake was washed with a mixture of toluene (22 Kg) and n-heptane (17 Kg), dried under vacuum at a temperature of 30 to 40° C. for about 15 hours to obtain the compound of formula (XII) (15.5 Kg, 81.2% yield, 100% assay, 100% purity) as an off-white solid.

### Example 10: Preparation and Synthesis of Compound of Formula (XIII) (2-Chloro-1-(6-(4-fluorobenzyl)-5-(hydroxymethyl)-3,3-dimethyl-2,3-dihydro-1H-pyrrolo[3,2-b]pyridine-1-yl)ethenone)

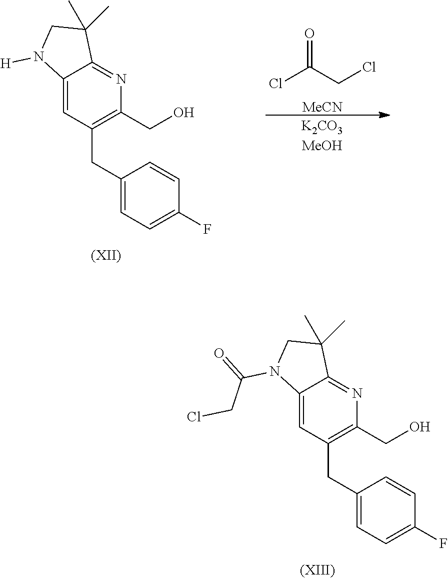

2-chloroacetyl chloride (3.04 Kg, 1.10 eq) was added to a mixture of the compound of formula (XII) (7.0 Kg, 100% assay, 100% purity, 1 eq) in acetonitrile (55 Kg) at a temperature of 5 to 10° C. The reaction mixture was warmed to a temperature of 7 to 13° C. and stirred at this temperature for 2 hours until residual compound of formula (XII) was NMT 0.2%. The reaction mixture was distilled down to 5 volumes under vacuum at NMT a temperature of 40° C. followed by the addition of toluene (30.45 Kg) and distilling down to 5 volumes. Methanol (66.5 Kg) was added and cooled to a temperature of 0 to 5° C. A solution of potassium carbonate (7.56 Kg, 2.24 eq) in water (42.8 Kg) was added at this temperature and agitated for approximately 30 minutes. The pH of the reaction mixture was adjusted to 3.5 to 4.5 using 3M hydrochloric acid (27 Lit). Toluene (2×30 Kg) was added and distilled to 10 volumes under vacuum at NMT a temperature of 20° C. Fresh toluene (60.9 Kg) was added and the organic layer was separated. The aqueous layer was washed with toluene (30 Kg). The combined organic layer was distilled to about 10 vol and cooled to a temperature of 0 to 5° C. n-Heptane (47.6 kg) was added and stirring was continued at this temperature for about 1 hour and filtered. The filter cake was washed with a mixture of toluene (6 Kg) and n-heptane (4.76 Kg) to obtain the compound of formula (XIII) (8.0 Kg, 90.8% yield; 99.3% assay, 99.76% purity).

### Example 11: Preparation and Synthesis of Compound of Formula (XV) (tert-butyl-(2R,5R)-5-(hydroxymethyl)-2-methylpiperazine-1-carboxylate)

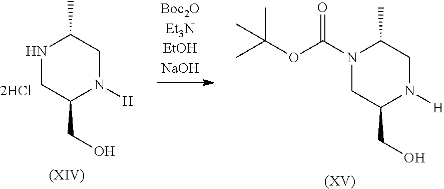

Triethyl amine (135.2 Kg, 3 eq) and di-tert-butyl dicarbonate (242.6 Kg, 2.5 eq) was added to a solution of the compound of formula (XIV) (98.8 Kg, 90.9% assay, 95% purity, 1 eq) in ethanol (287.5 Kg). The mixture was reacted at a temperature of 15 to 30° C. for 12 hours until reaction completion (residual compound of formula (XIV) was NMT 1%). A solution of sodium hydroxide (124.4 Kg, 30 eq) in water (362 Kg) was added, and the mixture was heated to a temperature of 40 to 45° C. for 30 mins, at a temperature of 50 to 60° C. for 30 mins and at a temperature of 70 to 75° C. for 30 hours until reaction completion. The reaction mixture was cooled to a temperature of 15 to 30° C., and the pH was adjusted to 9.0 to 9.5 using aqueous hydrochloric acid solution (181.4 Kg in 308 Kg of water). The mixture was filtered and washed with dichloromethane (718 Kg). The organic phase from the filtrate was separated, and the aqueous layer was extracted with dichloromethane (3×719 Kg). The combined organic layer was washed with brine (157 Kg of sodium chloride in 901 Kg of water) and concentrated to about 4 volumes under vacuum at NMT a temperature of 45° C. The solvent was swapped with methyl tert-butyl ether (2×336 Kg), distilling to about 4 volumes until residual dichloromethane was NMT 15%. The solvent was swapped with n-heptane (3×310 Kg) under vacuum at NMT 45° C., distilling down to about 6 volumes until residual dichloromethane was NMT 0.5%, residual methyl tert-butyl ether was NMT 3% and residual ethanol was NMT 0.5%. The mixture was cooled to a temperature of 10 to 20° C., stirred at this temperature for 2.5 hours and filtered. The filter cake was washed with n-heptane (126 Kg) and dried under a flow of nitrogen until residual n-heptane was NMT 0.5%, to obtain the compound of formula (XV) (88.4 Kg, 84.8% yield, 97.6% assay, 100% chemical purity and 100% chiral purity) as a white solid.

### Example 12: Preparation of Compound of Formula (XVI) (tert-butyl-(2R,5R)-4-benzyl-5-(hydroxymethyl)-2-methylpiperazine-1-carboxylate)

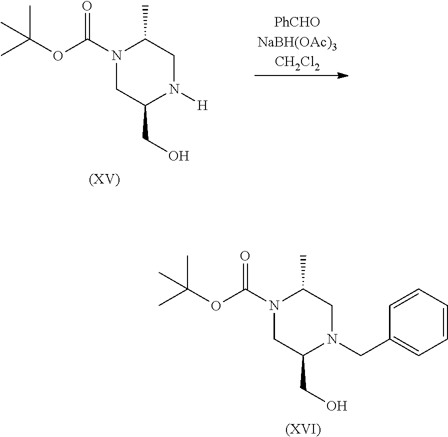

Benzaldehyde (46.8 Kg, 1.1 eq) was added to a solution of compound of formula (XV) (92.6 Kg, 97.6% assay, 100% purity, 1 eq) in dichloromethane (735.5 Kg) at a temperature of 15 to 30° C. The mixture was cooled to a temperature of 0 to 10° C., and sodium triacetoxyborohydride (111.2 Kg, 1.3 eq) was added. The reaction mixture was stirred at this temperature for 9.5 hours and then warmed to a temperature of 15-30° C. until reaction completion or until compound of formula (XV) reaches NMT 0.1%. The reaction mixture was quenched with a solution of sodium bicarbonate (75 Kg in 960 Kg water). The reaction mixture was degassed with a nitrogen gas purge and extracted with dichloromethane (617 Kg). The organic layer was separated and treated with a solution of sodium bisulfite (185 Kg in water 730 Kg) until benzaldehyde content was NMT 1%. The organic layer was washed with brine (300 Kg of sodium chloride in water, 993 Kg) and concentrated to about 6 volumes under reduced pressure at NMT 35° C. until a KF of NMT 0.2%. The resulting colorless clear solution of the compound of formula (XVI) in dichloromethane (259 Kg, 90% yield, 43.7% assay, 96% purity) is used in Example 14 to prepare compound of formula (XVII).

### Example 13: Preparation and Synthesis of the Oxalate Salt of Compound of Formula (XVIa)

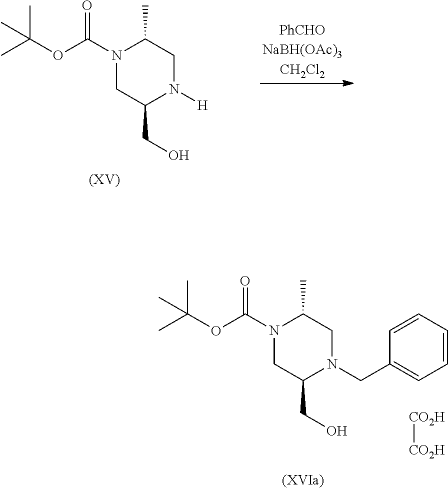

Alternatively, the compound of formula (XV) can be converted to oxalate salt compound of formula (XVIa) to be used in the next step in Example 14. To produce the compound of formula (XVIa), methyl tert-butyl ether (5 volumes) was slowly added to a solution of the compound of formula (XV) in dichloromethane (20 g, approx. 27.6% assay) and concentrated to 3 volume at a temperature of 30° C. The process was repeated 3 additional times to obtain a thin slurry with white solid suspended. The slurry was filtered and washed with methyl tert-butyl ether (3×5 mL). The combined organic filtrate was washed with saturated sodium bicarbonate (2×10 mL), brine (10 mL) and dried over anhydrous sodium sulfate. The suspension was filtered and rinsed with methyl tert-butyl ether (3×3 mL). The resulting solution was stirred at a temperature of 15 to 30° C., and a solution of oxalic acid (1.43 g, 15.9 mmol) in methyl tert-butyl ether (27.7 mL, 6 volume) was added slowly over 15 minutes. The white slurry obtained was stirred for 15 additional minutes at room temperature, filtered via a Buchner funnel and washed with methyl tert-butyl ether (2×2 volumes). The filter cake was dried under vacuum for 30 minutes to obtain an oxalate salt compound of formula (XVIa) (4.64 g, 11.3 mmol, 79% yield) as a white solid. The solid was pulped with methyl tert-butyl ether (10 volumes) and stirred for 15 minutes to result into a white slurry. The white slurry obtained was filtered via a Buchner funnel and washed with methyl tert-butyl ether (2×2 volumes). The white filter cake was dried under vacuum for 30 minutes to produce the oxalate salt compound of formula (XVIa) (4.43 g, 96% recovery, 100% purity) as a white solid. The oxalate salt compound of formula (XVIa) can also be used in the next step described in Example 14 to produce compound of formula (XVII).

### Example 14: Preparation and Synthesis of the Compound of Formula (XVII) (tert-butyl-(2R,5R)-4-benzyl-5-(chloromethyl)-2-methylpiperazine-1-carboxylate)

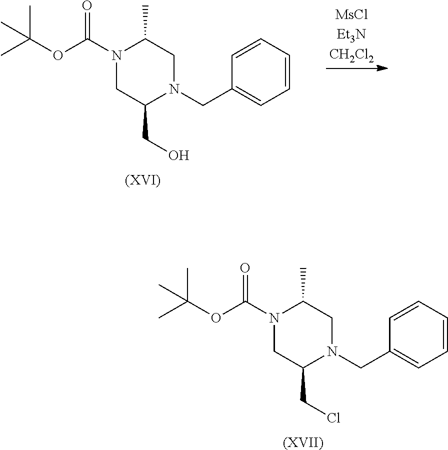

Additional dichloromethane (761 Kg) and triethyl amine (110 Kg, 3 eq) were charged to a solution of the compound of formula (XVI) (or its oxalate salt compound of formula (XVIa)) and cooled to a temperature of 0 to 10° C. Methane sulfonyl chloride (62.8 Kg, 1.5 eq) was added, and the reaction mixture was stirred at this temperature for 9.5 hours until a conversion of compound of formula (XVI) reached NMT 10%. The mixture was warmed to a temperature of 15 to 30° C. and stirred at this temperature for additional 5.5 hours until reaction completion or until the compound of formula (XVI) reached NMT 1%. The reaction was quenched with a solution of ammonium chloride (200 Kg) in water (588 Kg). The organic layer was separated, and the aqueous layer was extracted with dichloromethane (1013 Kg). The combined organic layer was washed with brine (312 Kg of sodium chloride in 931 Kg of water) and filtered through a pad of silica gel (93 Kg) using dichloromethane (1850 Kg). The filtrate was swapped with n-heptane (2×450 Kg) until residual dichloromethane reached NMT 0.2%. The mixture was cooled to a temperature of 0 to 5° C. and stirred at this temperature for 15 hours. The crystallized solid was filtered and washed with cold n-heptane (157 Kg) and dried under reduced pressure at NMT a temperature of 30° C. until residual n-heptane was NMT 0.5% and residual dichloromethane was NMT 0.5% to obtain the compound of formula (XVII) (99.0 Kg, 80.2% yield, 96.72% assay, 97.4% purity) as a light yellow solid.

### Example 15: Preparation and Synthesis of the Compound of Formula (XIX) (tert-butyl-(2R,5S)-4-benzyl-2-methyl-5-{[(3R)-3-methylmorpholin-4-yl]methyl}piperazine-1-carboxylate)

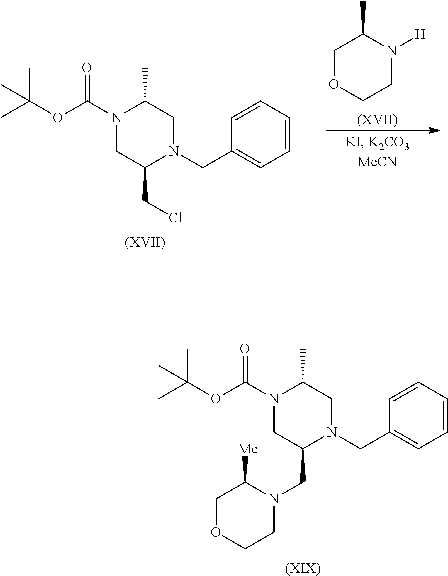

Potassium iodide (64.4 Kg, 1.9 eq), potassium carbonate (86 Kg, 3.1 eq) and (R)-3-methyl morpholine hydrochloride (compound of formula XVIII at 31 Kg, 1.07 eq) were added to a mixture of the compound for formula (XVII) (70.8 Kg, 96.7% assay, 97.4% purity, 1 eq) in acetonitrile (536 Kg) at a temperature of 15 to 30° C. The reaction mixture was heated to a temperature of 40 to 45° C. initially for about 30 mins followed by heating to a temperature of 57 to 62° C. The reaction mixture was heated at this temperature for about 7.5 hours until reaction completion or until the compound of formula (XVII) reached NMT 0.5% and was filtered to remove the inorganic residue. The filtrate was swapped with n-heptane (2×274 Kg) by distilling to about 6 volumes under reduced pressure at NMT a temperature of 45° C. until residual acetonitrile reached NMT 0.2%. The mixture was cooled to a temperature of −5 to 5° C. and stirred at this temperature for about 15 hours and filtered. The crude solid was dissolved in n-heptane (608 Kg) at a temperature of 15 to 30° C. and filtered through a pad of silica gel (40 Kg) using n-heptane (360 Kg) as rinse. The filtrate was concentrated to about 5 volumes, cooled to a temperature of −5 to 5° C. and maintained at this temperature for about 7 hours. The crystallized solid was filtered, washed with cold n-heptane (94 Kg) and dried under reduced pressure at NMT a temperature of 40° C. to obtain the compound of formula (XIX) (49.2 Kg, 60.6% yield, 100% assay, 99.9% purity) as a white solid.

### Example 16: Preparation and Synthesis of the Compound of Formula (XX) ((2R,5S)-tert-butyl-2-methyl-5-{[(R)-3-methylmorpholino]methyl}piperazine-1-carboxylate oxalate)

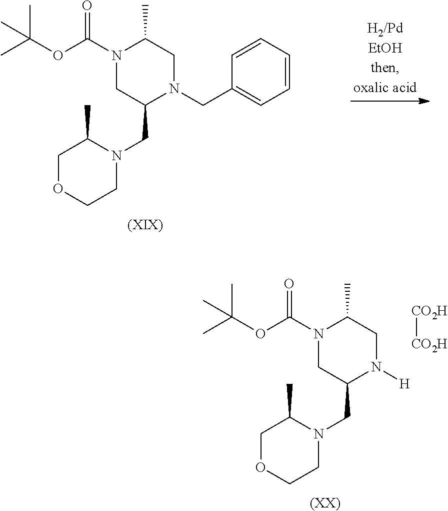

Palladium on carbon (0.65 Kg, 10% loading, 50% wet) was added to a mixture of the compound of formula (XIX) (13 Kg, 1 eq) in anhydrous ethanol (205 Kg). The reaction mixture was purged with nitrogen gas followed by hydrogen gas. The reaction mixture was pressurized to 2 bar and heated to a temperature of 65 to 75° C. for 3 hours. The reaction mixture was cooled to a temperature of 15 to 25° C., degassed with nitrogen gas, filtered and washed with ethanol (21 Kg). The filtrate was concentrated under reduced pressure at NMT a temperature of 50° C. to about 7.8 volumes and cooled to a temperature of 10 to 15° C. Oxalic acid (2.9 Kg, 1 eq) was added, and the reaction mixture was warmed to a temperature of 15 to 25° C. and stirred for 1 hour. Acetonitrile (159 Kg) was charged and stirred at this temperature for 40 minutes before cooling to a temperature of 0 to 5° C. The mixture was stirred at a temperature of 0 to 5° C. for 1 hour, filtered and washed with cold acetonitrile (2×40 Kg) and dried under reduced pressure at NMT a temperature of 50° C. to obtain the compound of formula (XX) in oxalate salt form (10.6 Kg, 81.5% yield, 99.88% purity) as a white solid.

### Example 17: Preparation and Synthesis of the Compound of Formula (XXI) ((2R,5S)-tert-butyl-4-(2-(6-(4-fluorobenzyl)-5-(hydroxymethyl)-3,3-dimethyl-2,3-dihydro-1H-pyrrolo[3,2-b]pyridine-1-yl)-2-oxoethyl)-2-methyl-5-(((R)-3-methylmorpholineo)methyl)piperazine-1-carboxylate)

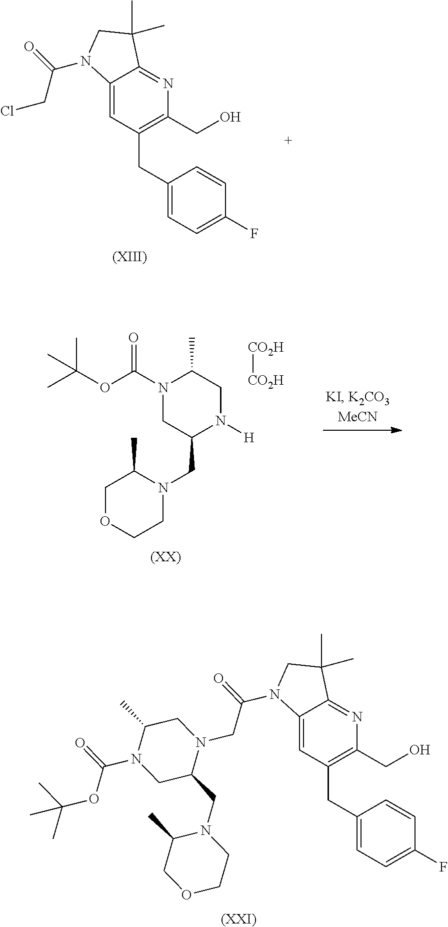

A mixture of the compound of formula (XIII) (7.9 Kg, 1 eq), the compound of formula (XX) (9.6 Kg, 1.1 eq), potassium iodide (7.1 Kg, 1.97 eq) and potassium carbonate (18.0 Kg, 5.94 eq) in acetonitrile (75 Kg) was stirred at a temperature of 15 to 25° C. for 3 hours until reaction completion or until the compound of formula (XIII) reached NMT 0.5%. The reaction mixture was distilled under reduced pressure at NMT a temperature of 40° C. to 4 volumes and cooled to a temperature of 15 to 25° C. Ethyl acetate (43 Kg) and water (63 Kg) were charged and stirred for 15 minutes. The organic layer was separated, and the aqueous layer was extracted with ethyl acetate (21 Kg). The combined organic layer was washed with a solution of potassium carbonate (3.2 Kg, 1.05 eq) in water (63 Kg) followed by three washes with a solution of potassium dihydrogen phosphate (3.6 Kg) and sodium chloride (3.6 Kg) in water (30 Kg). The organic layer was treated with Quadrasil MP® (0.40 Kg) at a temperature of 15 to 25° C. for 3 hours, filtered and washed with methanol (25 Kg). The filtrate was swapped with methanol (25 Kg, 63 Kg) by distillation under reduced pressure at NMT a temperature of 40° C. to 4 volumes. Anhydrous methanol (85 Kg) was charged to obtain a solution of the compound of formula (XXI) in methanol (115.7 Kg, 87% yield, 10.5% assay, 98.7% purity), which was used as is in the next step in Example 18 to prepare the compound of formula (XXII).

### Example 18: Preparation and Synthesis of the Compound of Formula (XXII) (1-(6-(4-fluorobenzyl)-5-(hydroxymethyl)-3,3-dimethyl-2,3-dihydro-1H-pyrrolo[3,2-b]pyridine-1-yl)-2-((2R,5R)-5-methyl-2-(((R)-3-methylmorpholino)methyl)piperazin-1-yl)ethenone)

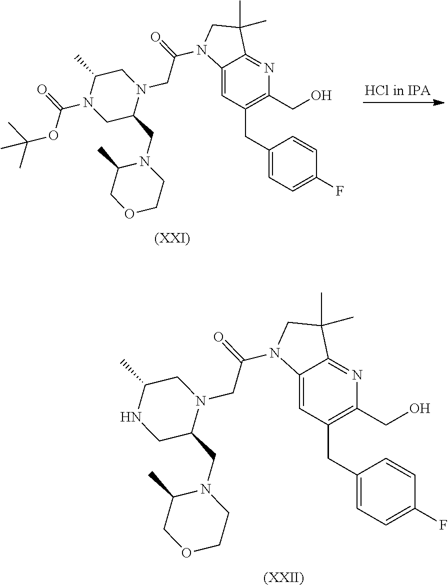

The solution of the compound of (XXI) in methanol (114.6 Kg, 10.5% assay, 98.7% purity, 1 eq) obtained from the previous step in Example 17 was cooled to a temperature of 0 to 10° C., and 6N hydrochloric acid in methanol (22 Kg, 7.7 eq) was added. The reaction mixture was warmed to a temperature of 15 to 25° C. and stirred at this temperature for 12 hours followed by 2 hours at a temperature of 30 to 40° C. until reaction completion or until the compound of formula (XXI) reached NMT 0.5%. The reaction mixture was distilled under reduced pressure at NMT a temperature of 40° C. to about 5 volumes. The solvent was swapped with water (2×48 Kg) followed by the addition of ethyl acetate (43 Kg). The organic phase was separated, and the aqueous phase containing the product was washed with ethyl acetate (43 Kg). The pH of the aqueous phase was adjusted to 11.5 to 12.0 using aqueous sodium hydroxide (37.6 Kg) followed by extraction with ethyl acetate (3×54 Kg). The combined organic layer was washed twice with brine (7 Kg in water 65 Kg). The organic layer was separated and azeotropically distilled to 9 volumes under reduced pressure at NMT a temperature of 40° C. using ethyl acetate (3×108 Kg) until the water content was NMT 0.7% to obtain the compound of formula (XXII) as a solution in ethyl acetate (100.7 Kg, 88.6% yield, 8.9% assay, 98.2% purity), which is used as was in the next step in Example 19 to prepare the compound of formula (XXIII).

### Example 19: Preparation and Synthesis of the Compound of Formula (XXIII) (1-{6-[4-fluorophenyl)methyl]-5-(hydroxymethyl)-3,3-dimethyl-1H-2H,3H-pyrrolo[3,2-b]pyridine-1-yl}-2-[(2R,5R)-5-methyl-2-{[(3R)-3-methylmorpholin-4-yl]methyl}piperazin-1-yl)ethen-1-one, L-(+)-lactic acid salt)

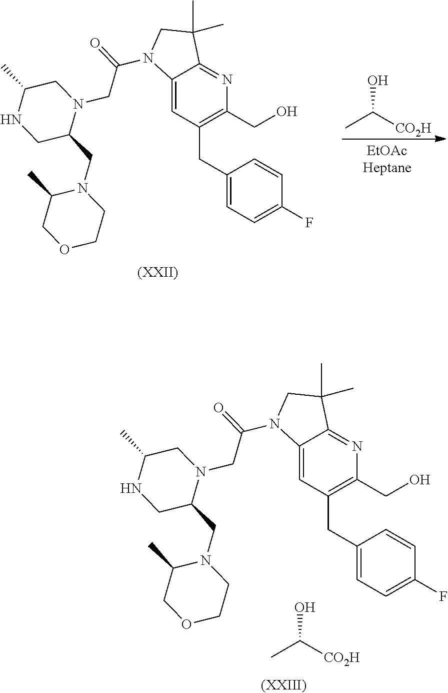

A solution of the compound of formula (XXII) (from the previous step in Example 18) in ethyl acetate (98.7 Kg, 8.9% assay, 98.2% purity) was distilled to 7.5 volumes under reduced pressure at NMT a temperature of 40° C. until the water content was NMT 0.7%. A solution of anhydrous L-(+)-lactic acid (1.62 Kg, 1.10 eq) in ethyl acetate (20 Kg) with a water content of NMT 0.7%, was clarified and rinsed with ethyl acetate (4.0 Kg). The solution of the compound of formula (XXII) in ethyl acetate was heated to a temperature of 40 to 50° C., and a ⅓ portion of the anhydrous lactic acid solution in ethyl acetate was added at this temperature. The mixture was seeded with the seed compounds of formula (XXIII) (44 g) at this temperature, and the remaining ⅔ portion of lactic acid solution in ethyl acetate was added slowly over 2 hours at a temperature of 40 to 50° C. n-Heptane (66 Kg) was added at this temperature and subjected to agitation for approximately 5.5 hours followed by cooling to a temperature of 5 to 15° C. with a cooling rate of 1° C. in 5 min. The resulting slurry was stirred at this temperature for at least 10 hours, filtered and washed with a mixture of n-heptane (12 Kg) and ethyl acetate (16 Kg). The filter cake was dried under reduced pressure at NMT a temperature of 45° C. until residual ethyl acetate was NMT 4500 ppm and residual n-heptane was NMT 4500 ppm to obtain the compound of formula (XXIII) as a lactic acid salt in crystalline white solid form (10.6 Kg, 100% yield, 99.58% purity; palladium content <1 ppm, Form C).

### Example 20: Experimental Data for Crystallization of the Compound of Formula (XXIII)

A batch of crude lactic acid salt of compound of formula (XXIII) was prepared by reacting compound of formula (XXIII) (37 g) in ethyl acetate (255 mL) with anhydrous L-(+)-lactic acid (1.1 eq). The resulting solution was evaporated to dryness to obtain the crude lactic acid salt of the compound of the formula (XXIII) (41.7 g, 96.5% yield, 98.81% area HPLC purity). This crude lactic acid salt of compound of the formula (XXIII) was used in the following crystallization studies.

Crystallization Experiment 1: Crystallization from Methyl Isobutyl Ketone (MIBK) and n-Heptane:

A suspension of the crude lactic acid salt compound of formula (XXIII) (4.0 g) in methyl isobutyl ketone (MIBK) (60 mL) was heated to a temperature of 80° C. for dissolution. The solution was cooled to a temperature of 60° C., seeded with the compound of formula (XXIII) and cooled to a temperature of 20° C. over 12 hours. n-Heptane (320 mL) was added over 1 hour. The resulting suspension was filtered and dried under vacuum to obtain a crystallized lactic acid salt compound of formula (XXIII) (68% yield, 99.16% area HPLC purity, XRPD: Form C).

Crystallization Experiment 2: Crystallization from Isopropanol and n-Heptane:

A suspension of the crude lactic acid salt compound of formula (XXIII) (4.0 g) in isopropanol (12 mL) was heated to a temperature of 30° C. for dissolution. n-Heptane (12 mL) was added over 2 hours. The resulting suspension was cooled to a temperature of 20° C. in 6 hours, filtered and dried under vacuum to obtain a crystallized lactic acid salt compound of formula (XXIII) (67% yield, 99.18% area HPLC purity, XRPD: Form C).

Crystallization Experiment 3: Crystallization from Methyl Ethyl Ketone (MEK) and n-Heptane:

A suspension of the crude lactic acid salt compound of formula (XXIII) (4.0 g) in methyl ethyl ketone (MEK) (18 mL) was heated to a temperature of 60° C. for dissolution. The solution was cooled to a temperature of 45° C., seeded with a lactic acid salt compound of formula (XXIII) and cooled to a temperature of 20° C. over 3 hours. n-Heptane (12 mL) was added over 6 hours. The resulting suspension was filtered and dried under vacuum to obtain a crystallized lactic acid salt compound of the formula (XXIII) (86% yield, 99.13% area HPLC purity, XRPD: Form C).

Crystallization Experiment 4: Crystallization from Tetrahydrofuran (THF) and n-Heptane:

A suspension of the crude lactic acid salt compound of formula (XXIII) (4.0 g) in tetrahydrofuran (THF) (9 mL) was heated to a temperature of 50° C. for dissolution. The solution was cooled to a temperature of 35° C. and n-heptane (2 mL), seeded with lactic acid salt seed compounds of formula (XXIII) and cooled to a temperature of 20° C. over 6 hours. n-Heptane (8 mL) was added over 6 hours. The resulting suspension was filtered and dried under vacuum to obtain a crystallized lactic acid salt compound of formula (XXIII) (81% yield, 99.23% area HPLC purity, XRPD: Form C).

Crystallization Experiment 5: Crystallization from Acetonitrile and Methyl Tert-Butyl Ether (MTBE):

A suspension of the crude lactic acid salt compound of formula (XXIII) (4.0 g) in acetonitrile (10 mL) was heated to a temperature of 57° C. for dissolution. The solution was cooled to a temperature of 50° C., seeded with lactic acid salt seed compounds of formula (XXIII). Methyl tert-butyl ether (20 mL) was added, and the solution was cooled to a temperature of 20° C. over 6 hours. The resulting suspension was filtered and dried under vacuum to obtain a crystallized lactic acid salt compound of formula (XXIII) (69% yield, 99.62% area HPLC purity, XRPD: Form C).

Crystallization Experiment 6: Preparation of a Lactate Salt Compound of Formula (XXIII) from Methyl Acetate and n-Heptane:

A solution of a free base compound of formula (XXIII) in ethyl acetate (17.47 g, 28.62% assay; 5 g of free base) was swapped (3 times) with methyl acetate (50 mL). A solution of L-(+)-lactic acid (0.92 g) in methyl acetate (72 mL) was added and the reaction mixture was heated to a temperature of 50° C. The solvent was switched (3 times) with methyl acetate (50 mL). The solution was adjusted to a temperature of 40° C. in 50 min and seeded with a lactate salt compound of formula (XXIII) (25 mg). The suspension was concentrated to 8 vol, n-heptane (40 mL) was added in 6 h, and the suspension was cooled to a temperature of 20° C. in 6 hours. The solid was filtered and dried at a temperature of 55° C. for 7 hours to obtain a crystallized lactate salt compound of formula (XXIII) (5.11 g, 88% yield; 99.67% area HPLC purity; Form C).

Crystallization Experiment 7: Preparation of a Lactate Salt Compound of the Formula (XXIII) from Ethyl Acetate and n-Heptane:

A solution of a free base compound of formula (XXIII) in ethyl acetate (17.47 g, 28.62% assay; 5 g of free base) was swapped (3 times) with ethyl acetate (50 mL) to obtain a KF<0.5%. A solution of L-(+)-lactic acid (0.92 g) in ethyl acetate (53 mL) was added, and the reaction mixture was heated to a temperature of 78° C. The solution was adjusted to a temperature of 65° C. in 50 min and seeded with lactate salt compound of formula (XXIII) (25 mg). The solution was adjusted to a temperature of 40° C. in 90 min, n-heptane (73 mL) was added in 6 hours, and the solution was cooled to a temperature of 20° C. in 6 hours. The solid was filtered and dried at a temperature of 55° C. for 7 hours to obtain a crystallized lactate salt compound of formula (XXIII) (5.4 g, 93% yield; 99.50% area HPLC purity; Form C).

X-Ray Powder Diffraction of Form C results for the crystallized end-product compound of formula (XXIII) described in Crystallization Experiment 7 are provided in FIG. 5.

Crystallization Experiment 8: Crystallization of Crude Lactic Acid from Ethyl Acetate and n-Heptane:

A suspension of crude lactic acid (11 Kg; 99.76% area HPLC purity) in ethyl acetate (158 Kg) was heated to reflux (at about 78° C.). The obtained solution is cooled to 65° C. and filtered to remove any insoluble material and finally rinsed with ethyl acetate (5 Kg). The resulting solution was heated to reflux (at about 78° C.) and cooled to 46-50° C. at the rate of 0.5° C./min). After seeding with lactate salt compound of formula (XXIII) (55 g) at 48° C., the mixture was maintained at this temperature for about 30 min. The slurry was then cooled to 20° C. in about 140 min and n-heptane (62 Kg) was added over 100 min. The slurry was then cooled to 10° C. in about 50 min and the mixture was stirred at this temperature for about 5 hours. The solid was filtered, washed with a mixture of ethyl acetate (20 Kg) and n-heptane (15 Kg), and dried under vacuum at 40-50° C. for about 4 hours to obtain crystallized lactate salt compound of formula (XXIII) (9.9 Kg, 90% yield; 99.91% area HPLC purity, Form C).

Based on experimental data, there is a correlation between the palladium content in the final end-product and drug substance of formula (XXIII) and the oxidative degradation of an aldehyde impurity. This correlation was observed at a storage condition of 25° C. and 60% relative humidity (RH). The degradation is manifested as an aldehyde impurity peak of formula (XXV) appearing at a relative retention time (RRT) of 1.3.

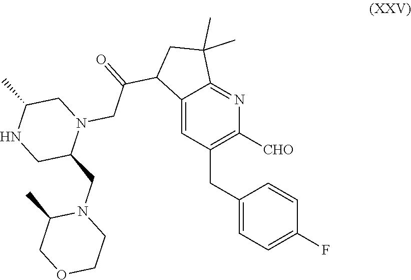 [SMILES: C[C@@H]1CN(CC(=O)C2CC(C)(C)c3nc(C=O)c(Cc4ccc(F)cc4)cc32)[C@@H](CN2CCOC[C@H]2C)CN1]

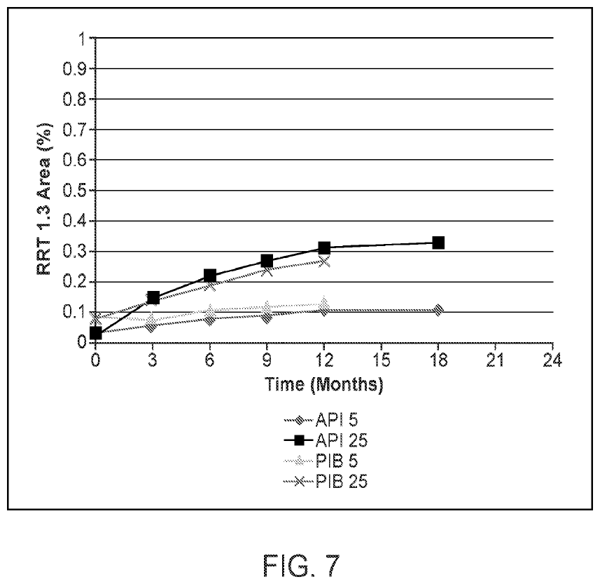

This impurity peak is found to be no more than 0.2% a/a when stored at 2-8° C., but higher levels are observed at elevated temperatures. The experimental results are depicted in FIGS. 6-7 and Tables 1, 4 and 5 for drug substance lots and corresponding formulations outlined in Tables 1-3.

The lots of compound of formula (XXIII) shown in Table 1 were used to obtain the experimental results.

TABLE 1
API Drug Substance Lots Manufactured

| Lot | RRT 1.3 | Pd |
|---|---|---|
| No. | Peak Area | Concentration |
| 1 | 0.13% | 6.5 ppm |
| 2 | 0.03% | 0.73 ppm |
| 3 | <0.1% | 1.9 ppm |
| 4 | 0.05% | 49 ppm |
| 5 | 0.05% | 50 ppm |
| 6 | <0.1% | <1 ppm |

TABLE 2
PIB Drug Product Batches Manufactured

| Batch No. | Lot Nos. Used | Strength |
|---|---|---|
| 1 | 1 | 200 mg |
| 2 | 2 | 400 mg |

TABLE 3
Capsule Drug Product Batches Manufactured

| Batch No. | Lot Nos. Used | Strength |
|---|---|---|
| 1 | 4 | 30 mg |
| 2 | 4 | 180 mg |
| 3 | 2 & 3 | 30 mg |
| 4 | 2 & 3 | 180 mg |

The stability data and effect of palladium content on aldehyde impurity represented by the area percent of RRT 1.3 at a storage condition of 25° C. and 60% RH is provided in Table 4 and FIG. 6.

TABLE 4
Stability Data for Area % of RRT 1.3 at 25° C./60% RH

| | Level of Impurity (Area %) at RRT | | |
|---|---|---|---|
| | 1.3 on Stability at 25° C./60% RH | | |
| | API Lot No. 1 | API Lot No. 2 | Capsule Batch 2 |
| Time | Pd content | Pd content | Pd content |
| (months) | 6.5 ppm | 0.72 ppm | 49 ppm in API |
| 0 | 0.13 | 0.03 | 0.09 |
| 3 | 0.18 | 0.15 | 0.37 |
| 6 | 0.26 | 0.22 | TBD |
| 9 | 0.33 | 0.27 | TBD |
| 12 | 0.38 | 0.31 | TBD |
| 18 | 0.45 | 0.33 | TBD |
| 24 | 0.54 | TBD | TBD |

TBD = To Be Determined

The effect of temperature on stability and the level of aldehyde impurity represented by the area percent of RRT 1.3 is provided in Table 5 and FIG. 7.

TABLE 5
Stability Data for Area % of RRT 1.3 at 5° C. and 25° C./60% RH

| | Effect of Temp on Level of | | | |
|---|---|---|---|---|
| | Impurity at RRT 1.3 (Area %) | | | |
| | | | PIB Drug Product | |
| Time | API Lot No. 2 | | Batch No. 2 | |
| (months) | 5° C. | 25° C. | 5° C. | 25° C. |
| 0 | 0.03 | 0.03 | 0.08 | 0.08 |
| 3 | 0.06 | 0.15 | 0.08 | 0.14 |
| 6 | 0.08 | 0.22 | 0.11 | 0.19 |
| 9 | 0.09 | 0.27 | 0.12 | 0.24 |
| 12 | 0.11 | 0.31 | 0.13 | 0.27 |
| 18 | 0.11 | 0.33 | TBD | TBD |

The experimental results depicted in FIGS. 6-7 and Tables 1, 4 and 5 confirm that there is correlation between the palladium content in the drug substance and the oxidative degradation observed at 25° C./60% RH as seen by the percentage of the peak area appearing at RRT 1.3. These impurity peak levels are found to be no more than 0.2% a/a when stored at 2-8° C., but higher levels were observed at elevated temperatures. These results suggest that the reduction of palladium and maximization of purity in the end-product compound of formula (XXIII) results in a more stable active pharmaceutical ingredient and drug formulation.

Novel intermediate compounds of formulas (Ia), (Va), (VII), (IX), (X) and (XI) are disclosed herein and can be used in the synthesis of the end-product compound of formula (XXIII) and other compounds of formula (XXIIIa). Intermediates of formulas (Ia), (Va), (VII), (IX), (X) and (XI) offer greater efficiency, predictability, isolation, purity and stability in the end-product and during the synthesis of the end-product compound of formula (XXIII). Problems of the known processes for the preparation of the compound of formula (XXIII) and other compounds of formula (XXIIIa) are addressed by the embodiments and examples of the present application.

Novel intermediate compounds of formulas (IX), (X), (XI), (XVI), (XVIa) and (XX) are disclosed herein and can be used in the synthesis of the end-product compound of formula (XXIII) and other compounds of formula (XXIIIa). Intermediate compounds of formulas (IX), (X), (XI), (XVI), (XVIa) and (XX) offer greater efficiency, predictability, isolation, purity and stability in the end-product and during the synthesis of the end-product compound of formula (XXIII). Problems of the known processes for the preparation of the compound of formula (XXIII) and other compounds of formula (XXIIIa) are addressed by the embodiments and examples of the present application.

The compound of formula (XXIII) synthesized by steps and intermediates disclosed herein can be formed into complexes, prodrugs or salt forms as disclosed in U.S. Pat. No. 9,783,538.

The compound of formula (XXIII) synthesized by steps and intermediates disclosed herein can be used to treat diseases and conditions disclosed in U.S. Pat. No. 9,783,538.

The compound of formula (XXIII) synthesized by steps and intermediates disclosed herein can be administered in accordance with the medicaments, pharmaceutical formulations, pharmaceutical compositions, dosage forms, excipients, therapeutic agents and dosage regimen disclosed in U.S. Pat. No. 9,783,538.

The compound of formula (XXIII) synthesized by steps and intermediates disclosed herein can be used in a medicament for the treatment of a diseases or condition, including cancer mediated by IAP. Cancers that can be treated include, but are not limited to, acute myelogenous leukemia (AML), T-cell lymphomas, B-cell lymphomas, diffuse large B-cell lymphoma (DLBCL), MALT lymphoma, head and neck cancer and cervical cancer.

The exemplary compounds, intermediates and methods of synthesis disclosed produce a key intermediate compound of formula (IX) and higher purity and stability in the compound of formula (XXIII), which is an effective IAP antagonist. The exemplary synthesis paths also utilize new chemical entities, such as the compounds of formula (Ia) to produce the compounds of formulas (IX) and (XXIII).

The exemplary compounds, intermediates and methods of synthesis disclosed produce key intermediate compounds of formulas (IX), (X), (XI), (XVI), (XVIa) and (XX) and higher purity and high stability in the compound of formula (XXIII), which is an effective IAP antagonist. The exemplary synthesis paths also utilize new chemical entities, such as the compounds of formulas (IX), (X), (XI), (XVIa) and (XX) to produce the compound of formula (XXIII).

What is claimed:

1. A process for preparing a compound of formula (XXIII) 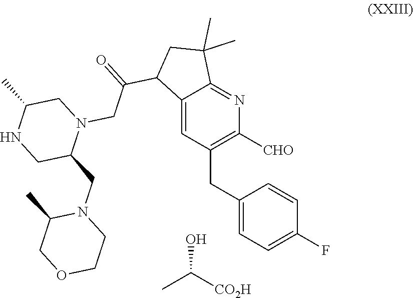
comprising
(i) contacting a compound of formula (XX) 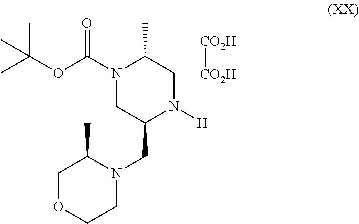
with a compound of formula (XIII) 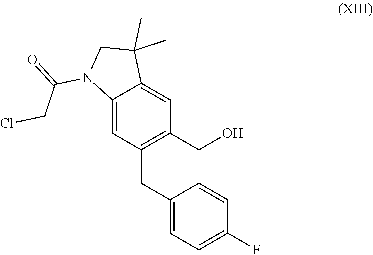 [SMILES: CC1(C)CN(C(=O)CCl)c2cc(Cc3ccc(F)cc3)c(CO)cc21]
under conditions sufficient to provide a compound of formula (XXI), or a salt, solvate or hydrate thereof, 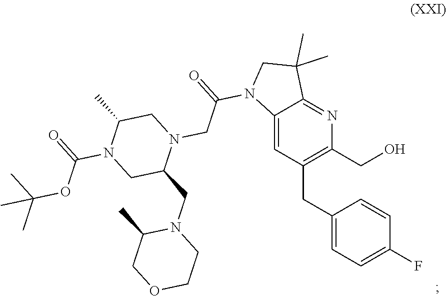 [SMILES: C[C@@H]1COCCN1C[C@H]1CN(C(=O)OC(C)(C)C)[C@H](C)CN1CC(=O)N1CC(C)(C)c2nc(CO)c(Cc3ccc(F)cc3)cc21]
(ii) deprotecting the compound of formula (XXI), or a salt, solvate or hydrate thereof, to provide a compound of formula (XXII), or a salt, solvate or hydrate thereof, 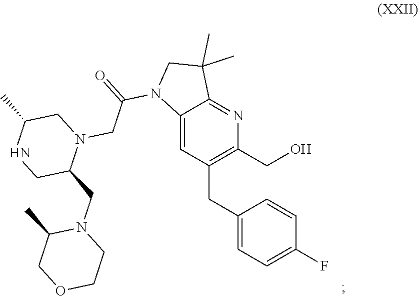 [SMILES: C[C@@H]1CN(CC(=O)N2CC(C)(C)c3nc(CO)c(Cc4ccc(F)cc4)cc32)[C@@H](CN2CCOC[C@H]2C)CN1]
and
(iii) contacting the compound of formula (XXII) with lactic acid to provide the compound of formula (XXIII).

2. The process of claim 1, wherein the compound of formula (XIII) is prepared by a process comprising
(i) reacting a compound of formula (V) 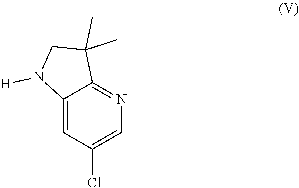
with a compound of formula (VI) 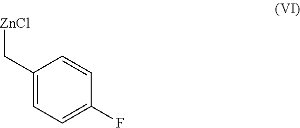
in the presence of one or more palladium catalysts and a ligand to provide a compound of formula (VII) 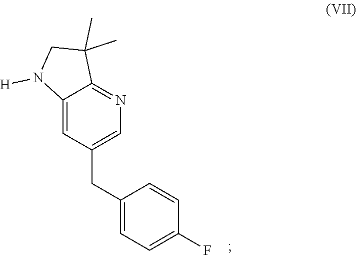 [SMILES: CC1(C)CNc2cc(Cc3ccc(F)cc3)cnc21]
(ii) brominating the compound of formula (VII) to obtain a compound of formula (VIII) 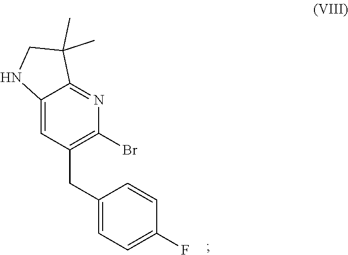 [SMILES: CC1(C)CNc2cc(Cc3ccc(F)cc3)c(Br)nc21]
(iii) protecting the compound of formula (VIII) to provide a compound of formula (IX) 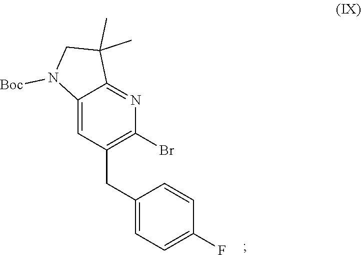 [SMILES: CC(C)(C)OC(=O)N1CC(C)(C)c2nc(Br)c(Cc3ccc(F)cc3)cc21]
(iv) contacting the compound of formula (IX) with carbon monoxide under conditions sufficient to provide the compound of formula (X), a salt, solvate or hydrate thereof. 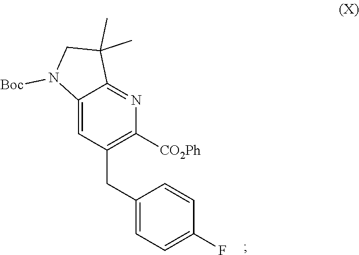
(v) removing the tert-butyloxy carbonyl (Boc) protecting group from a compound of formula (X), or a salt, solvate or hydrate thereof, to provide a compound of formula (XI) 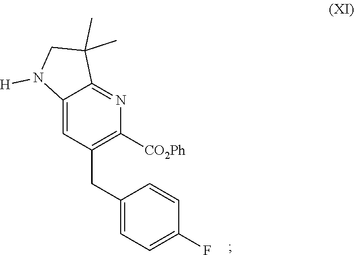
(vi) reducing the compound of formula (XI) to provide a compound of formula (XII) 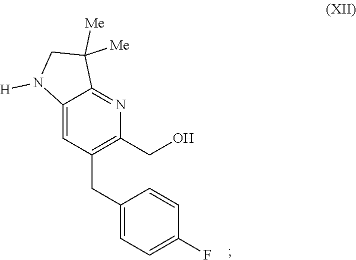 [SMILES: CC1(C)CNc2cc(Cc3ccc(F)cc3)c(CO)nc21]
and
(vii) contacting the compound of formula (XII) with chloroacetyl chloride to provide the compound of formula (XIII).

3. The process of claim 2, wherein the one or more palladium catalysts in step (i) are selected from the group consisting of a XPhos-Pd-G2 catalyst, Pd(OAc)₂ and Pd₂(dba)₃.

4. The process of claim 2, wherein the ligand in step (i) is selected from the group consisting of PPh₃, Xantphos, DPPE, DPPP, DPPB, DPPF, rac-BINAP, RuPhos, XPhos and tBuXphos.

5. The process of claim 2, wherein the conditions in step (iv) comprise reacting the compound of formula (IX) with (i) phenyl formate or phenol and (ii) carbon monoxide, in the presence of a palladium catalyst to produce the compound of formula (X).

6. The process of claim 5, wherein reacting the compound of formula (IX) occurs in solution in the presence of rac-1,1′-binaphthyl-2,2′-diphenyl phosphene.

7. The process of claim 5, wherein the palladium catalyst in step (iv) is selected from the group consisting of XPhos-Pd-G2 catalyst, Pd(OAc)₂ and Pd₂(dba)₃.

8. The process of claim 5, wherein reacting the compound of formula (IX) occurs in the presence of a ligand selected from the group consisting of PPh₃, Xantphos, DPPE, DPPP, DPPB, DPPF, rac-BINAP, RuPhos, tBuXphos and 2-dicyclohexylphosphino-2′,4′,6′-triisopropylbiphenyl (XPhos).

9. A process of preparing a compound of formula (XX) 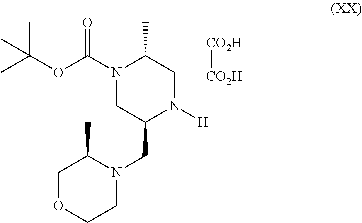
comprising
(i) debenzylating a compound of formula (XIX) 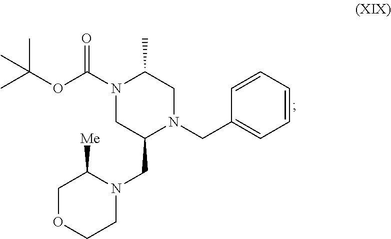 [SMILES: C[C@@H]1COCCN1C[C@H]1CN(C(=O)OC(C)(C)C)[C@H](C)CN1Cc1ccccc1]
and
(ii) contacting the debenzylated product with oxalic acid in a solvent to provide the compound of formula (XX).

10. The process of claim 9, wherein the debenzylation in step (i) is carried out in the presence of palladium on carbon and hydrogen gas.

11. A process for preparing a compound of formula (X), or a salt, solvate or hydrate thereof, 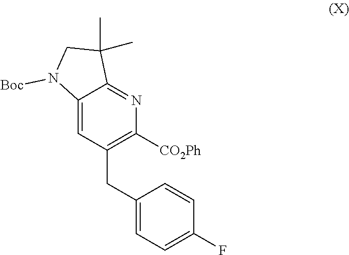
comprising contacting a compound of formula (IX) 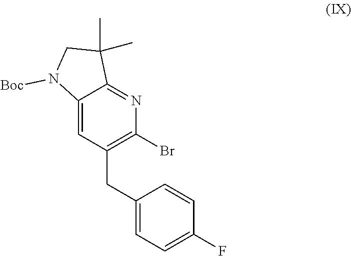 [SMILES: CC(C)(C)OC(=O)N1CC(C)(C)c2nc(Br)c(Cc3ccc(F)cc3)cc21]
with carbon monoxide under conditions sufficient to provide the compound of formula (X), a salt, solvate or hydrate thereof.

12. The process of claim 11, wherein the conditions comprise reacting the compound of formula (IX) with (i) phenyl formate or phenol and (ii) carbon monoxide, in the presence of a palladium catalyst to produce the compound of formula (X).

13. A process for preparing a compound of formula (XI), or a salt, solvate or hydrate thereof, 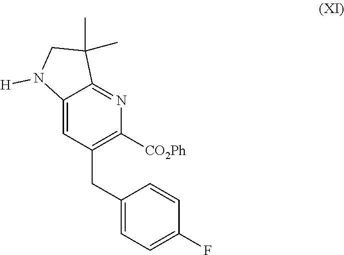
comprising removing the tert-butyloxy carbonyl protecting group from a compound of formula (X), or a salt, solvate or hydrate thereof, 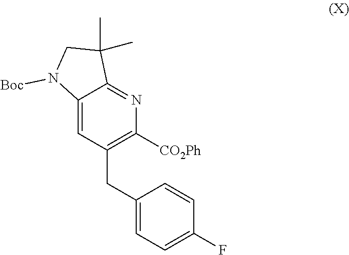
under conditions sufficient to provide the compound of Formula (XI), or a salt, solvate or hydrate thereof.

14. The process of claim 13 further comprising
(i) reducing the compound of formula (XI) under conditions sufficient to provide a compound of formula (XII) 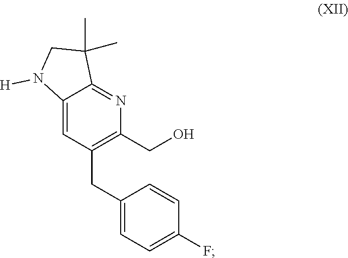 [SMILES: CC1(C)CNc2cc(Cc3ccc(F)cc3)c(CO)nc21]
and
(ii) contacting the compound of formula (XII) with 2-chloroacetyl chloride to provide a compound of formula (XIII)

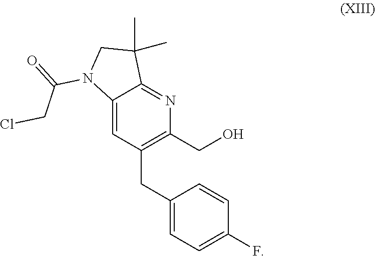 [SMILES: CC1(C)CN(C(=O)CCl)c2cc(Cc3ccc(F)cc3)c(CO)nc21]

15. The process of claim 14, wherein the reducing is conducted in the presence of lithium borohydride, sodium borohydride, lithium aluminum hydride, borane, sodium triacetoxy borohydride, L-selectride, K-selectride, Red-Al or DIBAL.

16. The process of any one of claims 14-15, wherein step (ii) is conducted at a temperature of about of −10° C. to about 0° C.

17. A process for preparing a compound of formula (XVIa) 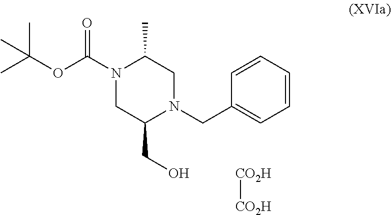 [SMILES: C[C@@H]1CN(Cc2ccccc2)[C@@H](CO)CN1C(=O)OC(C)(C)C ; O=C(O)C(=O)O]
comprising contacting a compound of formula (XVI) 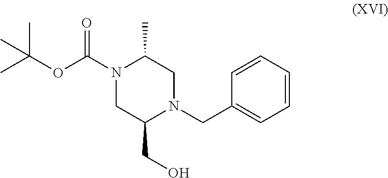 [SMILES: C[C@@H]1CN(Cc2ccccc2)[C@@H](CO)CN1C(=O)OC(C)(C)C]
with oxalic acid in a solvent to provide a compound of formula (XVIa).

18. The process of claim 17, wherein the solvent is methyl tert butyl ether (MTBE).

19. A compound of formula (XXIII) 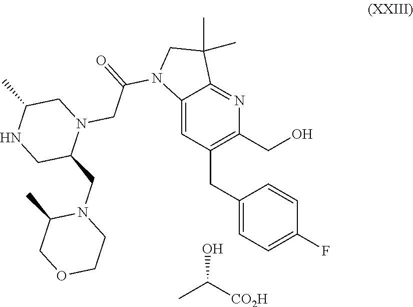
having a purity of at least 95%.

20. The compound of claim 19, having a purity of at least 98%.

21. The compound of any one of claims 19-20, wherein when the compound of formula (XXIII) is stored at 25° C. and 60% relative humidity for about 6 months, the compound of formula (XXIII) comprises no more than about 0.2% a/a of a compound of formula (XXV)

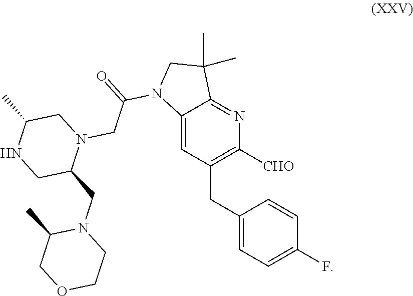 [SMILES: C[C@@H]1CN(CC(=O)N2CC(C)(C)c3nc(C=O)c(Cc4ccc(F)cc4)cc32)[C@@H](CN2CCOC[C@H]2C)CN1]

22. The compound of formula (XXIII) of any one of claims 19-20, wherein, when the compound of formula (XXIII) is stored at 25° C. and 60% relative humidity for about 12 months, the compound of formula (XXIII) comprises no more than about 0.3% a/a of a compound of formula (XXV)

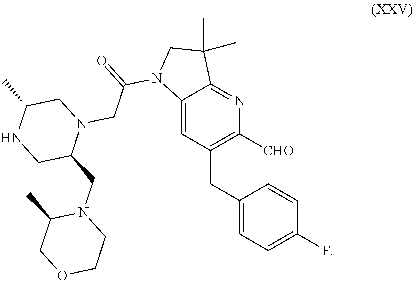 [SMILES: C[C@@H]1CN(CC(=O)N2CC(C)(C)c3nc(C=O)c(Cc4ccc(F)cc4)cc32)[C@@H](CN2CCOC[C@H]2C)CN1]

23. The compound of any one of claims 19-22, wherein the compound of formula (XXIII) comprises no more than about 50 ppm of palladium.

24. A composition comprising a compound of formula (XXIII) 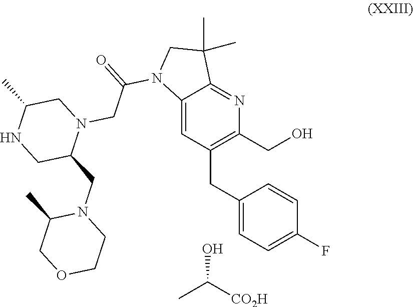
wherein at least 95% of the compound of formula (XXIII) is in Form C.

25. The composition of claim 24, wherein Form C of formula (XXIII) has an XRPD substantially as shown in FIG. 5.

26. A process for preparing a compound of formula (IX), or a salt, solvate or hydrate thereof, 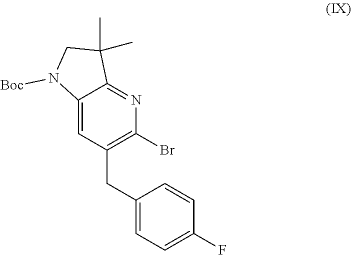 [SMILES: CC(C)(C)OC(=O)N1CC(C)(C)c2nc(Br)c(Cc3ccc(F)cc3)cc21]
comprising
(i) borylating a compound of formula (III), or a salt, solvate or hydrate thereof, 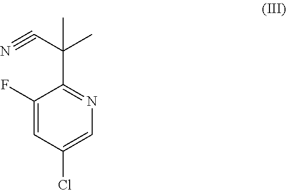
under conditions sufficient to provide a compound of formula (IIIa) 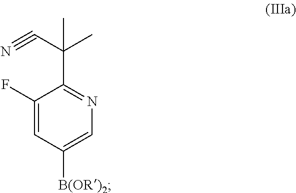
(ii) contacting the compound of formula (IIIa) with 4-fluorobenzyl chloride or 4-fluoro benzyl bromide under conditions sufficient to provide a compound of formula (IIIb), or a salt, solvate or hydrate thereof, 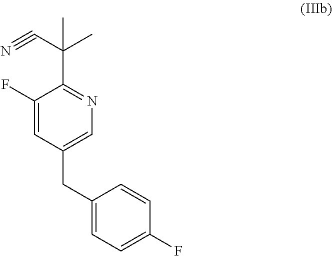 [SMILES: CC(C)(C#N)c1ncc(Cc2ccc(F)cc2)cc1F]
(iii) contacting the compound of formula (IIIb) with a reducing agent to provide a compound of formula (IIIc), or a salt, solvate or hydrate thereof, 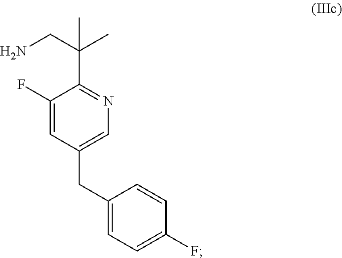 [SMILES: CC(C)(CN)c1ncc(Cc2ccc(F)cc2)cc1F]
(iv) cyclizing the compound of formula (IIIc) to provide a compound of formula (VII), or a salt, solvate or hydrate thereof, 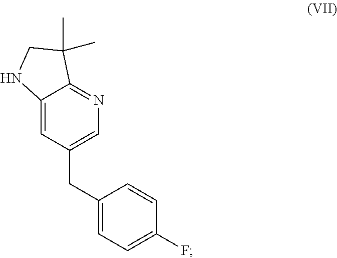 [SMILES: CC1(C)CNc2cc(Cc3ccc(F)cc3)cnc21]
(v) brominating the compound of formula (VII) to provide a compound of formula (VIII), or a salt, solvate or hydrate thereof, 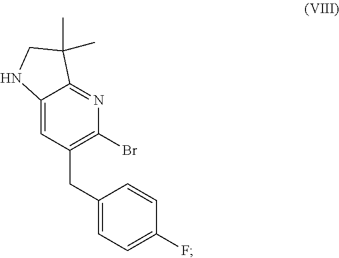 [SMILES: CC1(C)CNc2cc(Cc3ccc(F)cc3)c(Br)nc21]
and
(vi) protecting the compound of formula (VIII) with a tert-butyloxy carbonyl group to provide the compound of formula (IX), or a salt, solvate or hydrate thereof.

27. A process for preparing a compound of formula (IX), or a salt, solvate or hydrate thereof, 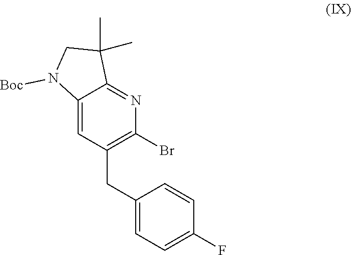 [SMILES: CC(C)(C)OC(=O)N1CC(C)(C)c2nc(Br)c(Cc3ccc(F)cc3)cc21]
comprising
(i) protecting a compound of formula (V), or a salt, solvate or hydrate thereof, 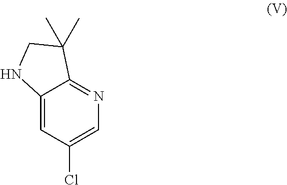
with a tert-butyloxy carbonyl group to provide a compound of formula (Va), or a salt, solvate or hydrate thereof, 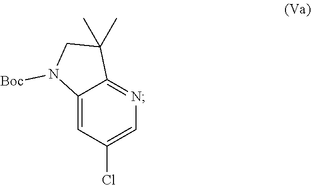 [SMILES: CC(C)(C)OC(=O)N1CC(C)(C)c2ncc(Cl)cc21]
(ii) borylating the compound of formula (Va), or a salt, solvate or hydrate thereof, to obtain a compound of formula (Vb), or a salt, solvate or hydrate thereof, 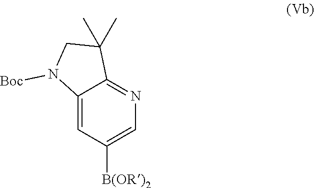
wherein each R′ is independently H, alkyl, or aryl group, or two alkyl or two aryl groups together with the atoms to which they are attached form a dioxaborolanyl ring;
(iii) contacting the compound of formula (Vb), with 4-fluorobenzyl chloride or 4-fluoro benzyl bromide under conditions sufficient to provide a compound of formula (Vc), or a salt, solvate or hydrate thereof, 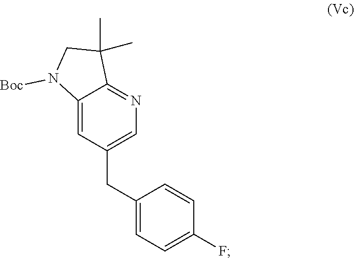 [SMILES: CC(C)(C)OC(=O)N1CC(C)(C)c2ncc(Cc3ccc(F)cc3)cc21]
and
(iv) brominating the compound of formula (Vc) to provide the compound of formula (IX), or a salt, solvate or hydrate thereof.

28. A compound of formula (Ia), or a salt, solvate or hydrate thereof, 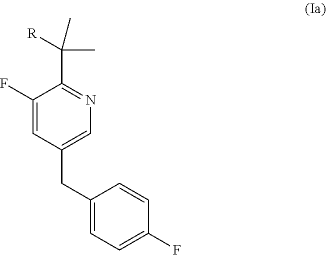
wherein R is CN or CH₂NH₂.

29. A compound of formula (I), or a salt, solvate or hydrate thereof, 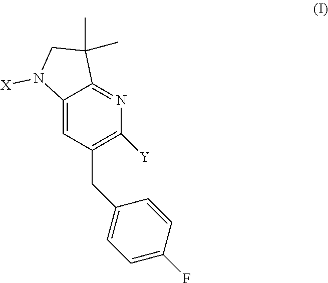
wherein X is H or a protecting group;
Y is COR; and
R is OH, O-alkyl or O-aryl.

30. A compound of formula (XVIa):

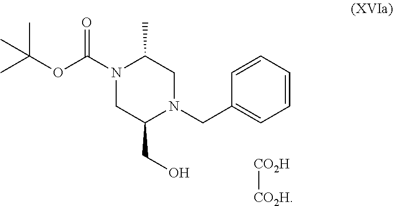

31. A compound of formula (IX), or a salt, solvate or hydrate thereof,

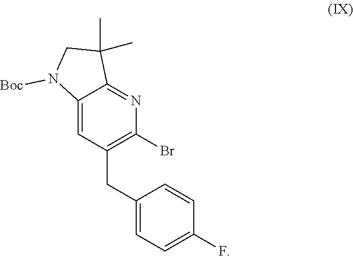 [SMILES: CC(C)(C)OC(=O)N1CC(C)(C)c2nc(Br)c(Cc3ccc(F)cc3)cc21]

32. A compound of formula (X), or a salt, solvate or hydrate thereof,

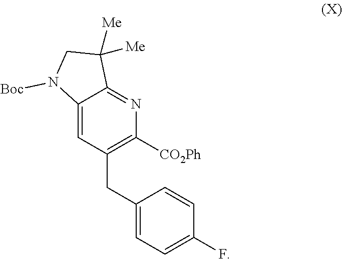

33. A compound of formula (XI), or a salt, solvate or hydrate thereof,

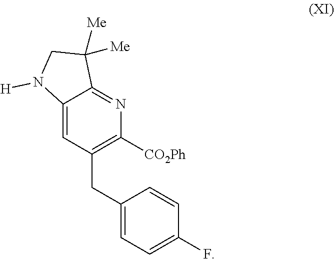

34. A compound of formula (XX):

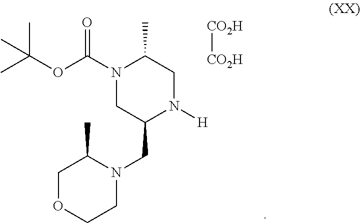

35. A compound of formula (IIIa): 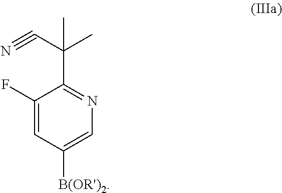
wherein each R′ is independently H, alkyl, or aryl group, or two alkyl or two aryl groups together with the atoms to which they are attached form a dioxaborolanyl ring.

36. A compound of formula (Vb): 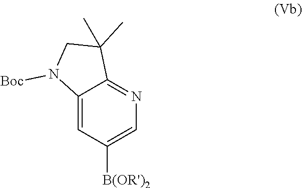
wherein each R′ is independently H, alkyl, or aryl group, or two alkyl or two aryl groups together with the atoms to which they are attached form a dioxaborolanyl ring.

37. A process for preparing a compound of formula (XXIII) 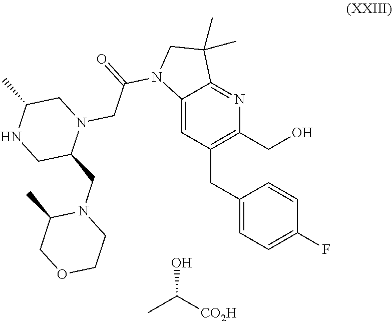
comprising
(i) contacting a compound of formula (IX), or a salt, solvate or hydrate thereof, 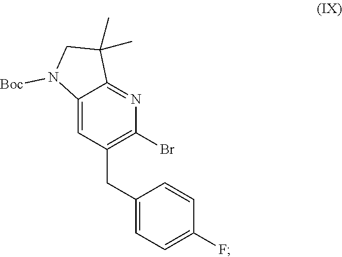 [SMILES: CC(C)(C)OC(=O)N1CC(C)(C)c2nc(Br)c(Cc3ccc(F)cc3)cc21]
with carbon monoxide under conditions sufficient to provide the compound of formula (X), a salt, solvate or hydrate thereof 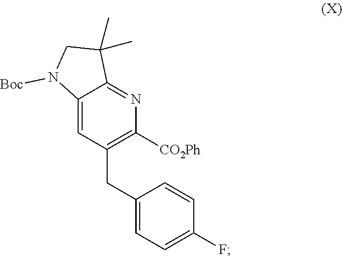
(ii) removing the tert-butyloxy carbonyl protecting group from the compound of formula (X), or a salt, solvate or hydrate thereof, to provide a compound of formula (XI), or a salt, solvate or hydrate thereof, 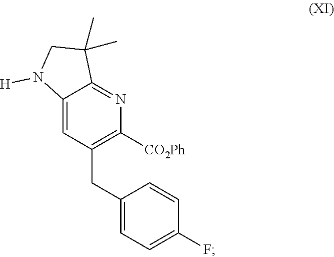
(iii) reducing the compound of formula (XI), or a salt, solvate or hydrate thereof, to provide a compound of formula (XII), or a salt, solvate or hydrate thereof, 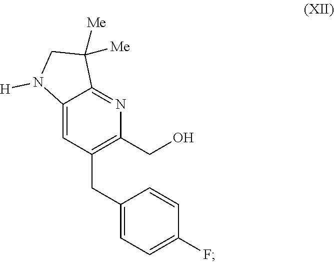 [SMILES: CC1(C)CNc2cc(Cc3ccc(F)cc3)c(CO)nc21]
(iv) contacting the compound of formula (XII), or a salt, solvate or hydrate thereof, with chloroacetyl chloride to provide the compound of formula (XIII), or a salt, solvate or hydrate thereof, 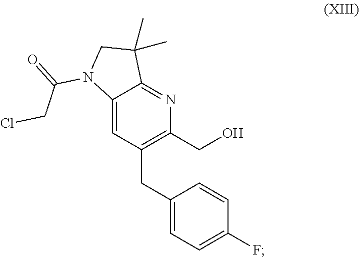 [SMILES: CC1(C)CN(C(=O)CCl)c2cc(Cc3ccc(F)cc3)c(CO)nc21]
(v) contacting the compound of formula (XIII), or a salt, solvate or hydrate thereof, with a compound of formula (XX) 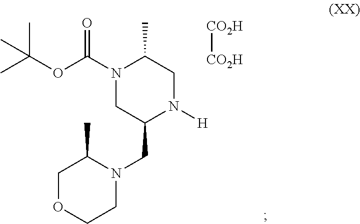
under conditions sufficient to provide a compound of formula (XXI), or a salt, solvate or hydrate thereof, 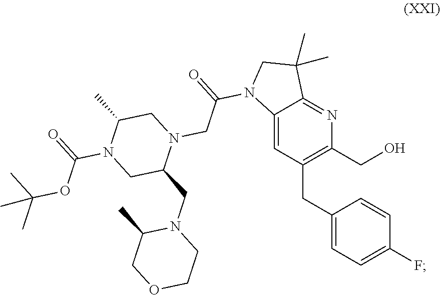 [SMILES: C[C@@H]1COCCN1C[C@H]1CN(C(=O)OC(C)(C)C)[C@H](C)CN1CC(=O)N1CC(C)(C)c2nc(CO)c(Cc3ccc(F)cc3)cc21]
and
(vi) deprotecting the compound of formula (XXI), or a salt, solvate or hydrate thereof, to provide a compound of formula (XXII), or a salt, solvate or hydrate thereof, 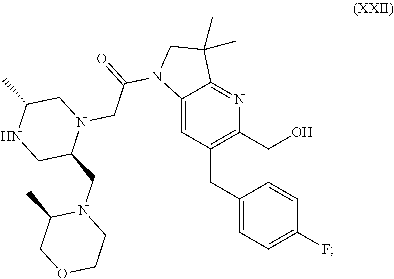 [SMILES: C[C@@H]1CN(CC(=O)N2CC(C)(C)c3nc(CO)c(Cc4ccc(F)cc4)cc32)[C@@H](CN2CCOC[C@H]2C)CN1]
and
(vii) contacting the compound of formula (XXII) with lactic acid to provide the compound of formula (XXIII).

38. A compound of formula (XXIII) 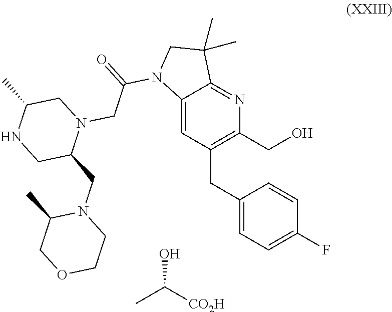
produced by the method of the claim 1 or claim 37.
